(19) United States
(12) Patent Application Publication
(10) Pub. No.: US 2025/0242054 A1
(43) Pub. Date: Jul. 31, 2025

(54) COMPOSITIONS AND METHODS FOR TREATING SENSORINEURAL HEARING LOSS, VESTIBULAR DYSFUNCTION AND VISION LOSS USING PROTOCADHERIN 15 DUAL VECTOR SYSTEMS

(71) Applicants: University of Maryland, Baltimore, Baltimore, MD (US); LIONS EYE INSTITUTE LIMITED, Nedlands (AU)

(72) Inventors: Zubair M. Ahmed, Ellicott City, MD (US); Saima Riazuddin, Ellicott City, MD (US); Saumil Sethna, San Diego, CA (US); Livia dos Santos Carvalho, Subiaco (AU)

(21) Appl. No.: 18/854,650

(22) PCT Filed: Apr. 6, 2023
(86) PCT No.: PCT/US2023/065484

§ 371 Date: Oct. 7, 2024

Related U.S. Application Data
(60) Provisional application No. 63/328,531, filed on Apr. 7, 2022.

(51) Int. Cl. A61K 48/00; A61K 9/00; A61K 38/17; A61P 27/02; A61P 27/16; C12N 15/86

(52) U.S. Cl. CPC A61K 48/005; A61K 9/0048; A61K 38/1709; A61K 48/0075; A61P 27/02; A61P 27/16; C12N 15/86; C12N 2750/14143; C12N 2800/40; C12N 2830/50

(57) ABSTRACT

Provided herein are dual vector systems for expressing a Protocadherin-15 protein or variant thereof in a subject, wherein the dual vector system comprises a first vector comprising a first coding polynucleotide that encodes an N-terminal portion of the Protocadherin-15 protein or variant thereof; and a second vector comprising a second coding polynucleotide that encodes a C-terminal portion of the Protocadherin-15 protein or variant thereof, wherein the first coding polynucleotide and the second coding polynucleotide that encode the Protocadherin-15 protein or variant thereof do not overlap.

### FIELD OF THE INVENTION

The field of the invention relates to pharmaceuticals and medicine, particularly compositions and methods for treating hearing loss, vision loss and vestibular dysfunction.

### BACKGROUND OF THE INVENTION

Usher syndrome type I (USH1) is characterized by congenital deafness, vestibular areflexia, and progressive retinal degeneration with age. The protein-truncating p.Arg245* founder variant of PCDH15 has an ˜2% carrier frequency among Ashkenazi Jews, accounting for nearly 60% of their USH1 cases. Longitudinal ocular phenotyping in thirteen USH1F individuals harboring the p.Arg245* variant revealed progressive retinal degeneration, leading to severe loss of vision with macular atrophy by the sixth decade. Mice homozygous for p.Arg250* (Pcdh15^{R250X}; equivalent to human p.Arg245*) also have early visual deficits evaluated using electroretinography, hearing loss as well as vestibular dysfunction. Sethna et al. (https://doi.org/10.1101/2021.06.08.447565).

Loss of vision in individuals with USH1, an autosomal recessive disorder, begins towards the end of their first decade of life due to retinitis pigmentosa (RP), eventually leading to near total blindness. Night blindness is an early sign in USH1 subjects followed by constriction of the visual field (tunnel vision) and finally clinical blindness (Vernon, 1969). Characteristic fundus features include pigmentary retinopathy, narrowing of the retinal vessels, and a pale appearance of the optic disk (Toms, Pagarkar et al., Ther Adv Ophthalmol 12:2515841420952194 (2020)).

Vestibular dysfunction in USH1 manifests as a delay in development of independent ambulation while hearing loss is usually severe to profound, congenital and sensorineural (Ahmed, Riazuddin et al., Clin Genet 63:431-44 (2003), Smith, Berlin et al., American Journal of Medical Genetics 50:32-8 (1994)). Cochlear implants can restore auditory perception in USH1 patients (Brownstein, Ben-Yosef et al., Pediatr Res 55:995-1000 (2004), Pennings, Damen et al., Laryngoscope 116:717-22 (2006)), but presently there is no effective treatment for the vision loss due to retinitis pigmentosa. Moreover, there is a lack of longitudinal data for the natural history of ocular abnormalities associated with variants of PCDH15 in humans. Only anecdotal clinical data has been reported thus far (Ahmed, Riazuddin et al., American Journal of Human Genetics 69:25-34 (2001), Ben-Yosef, Ness et al., New England Journal of Medicine 348:1664-1670 (2003), Brownstein, Ben-Yosef et al., Pediatric Research 55:995 (2004), Jacobson, Cideciyan et al., Human Molecular Genetics 17:2405-2415 (2008)).

Protocadherin-15 is a member of a large cadherin superfamily of calcium-dependent cell-cell adhesion molecules (Ahmed, Goodyear et al., J Neurosci 26:7022-34 (2006), Ahmed, Riazuddin et al., Am J Hum Genet 69:25-34 (2001), van Roy, Nature Reviews Cancer 14:121-134 (2014)). Within the vertebrate inner ear, protocadherin-15 is required for the structural maintenance and the mechanotransduction function of the sensory hair cells (Ahmed, Goodyear et al., The Journal of Neuroscience 26:7022-7034 (2006), Kazmierczak, Sakaguchi et al., Nature 449:87-91 (2007)). In the retina, protocadherin-15 is localized to the outer limiting membrane of photoreceptors (PR) and in Müller glia (Reiners, van Wijk et al., Hum Mol Genet 14:3933-43 (2005), van Wijk, van der Zwaag et al., Hum Mol Genet 15:751-65 (2006)). A reduction of ERG a- and b-waves amplitudes (˜40%) at 5 weeks of age in at least two Pcdh15 alleles in mice (Pcdh15^{av-5J} and Pcdh15^{av-jfb}) has been reported (Haywood-Watson, Ahmed et al., Investigative Ophthalmology & Visual Science 47:3074-3084 (2006)). However, the exact molecular function of protocadherin-15 in the retina remains elusive.

This background information is provided for informational purposes only. No admission is necessarily intended, nor should it be construed, that any of the preceding information constitutes prior art against the present invention.

### SUMMARY

It is to be understood that both the foregoing general description of the embodiments and the following detailed description are exemplary, and thus do not restrict the scope of the embodiments.

In one aspect, the invention provides a dual vector system for expressing a Protocadherin-15 protein or variant thereof in a subject, wherein the Protocadherin-15 protein comprises SEQ ID NO:2, wherein the dual vector system comprises
i) a first vector comprising a first coding polynucleotide that encodes an N-terminal portion of the Protocadherin-15 protein or variant thereof; and
ii) a second vector comprising a second coding polynucleotide that encodes a C-terminal portion of the Protocadherin-15 protein or variant thereof,
wherein the first coding polynucleotide and the second coding polynucleotide that encode the Protocadherin-15 protein or variant thereof do not overlap.

In another aspect, the invention provides a dual vector system for expressing a Protocadherin-15 protein or variant thereof in a subject, wherein the Protocadherin-15 protein comprises SEQ ID NO:4, wherein the dual vector system comprises
i) a first vector comprising a first coding polynucleotide that encodes an N-terminal portion of the Protocadherin-15 protein; and
ii) a second vector comprising a second coding polynucleotide that encodes a C-terminal portion of the Protocadherin-15 protein,
wherein the first coding polynucleotide and the second coding polynucleotide that encode the Protocadherin-15 protein or variant thereof do not overlap.

In some embodiments, the variant comprises an amino acid sequence that is at least 90% identical to SEQ ID NO:2 or 4.

In some embodiments, the first and second vectors are adeno-associated viral (AAV) vectors.

In some embodiments, the first and/or second vector are adeno-associated viral (AAV) vectors selected from AAV2/2, AAV2/5, AAV2/9, AAV2/Anc80, AAV-7m8, AAV8, and R100.

In some embodiments, the first vector comprises a promoter operably linked to the first coding polynucleotide.

In some embodiments, the promoter is selected from a CMV promoter, a CAG promotor, and a tissue specific promoter.

In some embodiments, the promoter is a CMV promoter having a polynucleotide sequence comprising at least 80% sequence identity to SEQ ID NO:7.

In some embodiments, the first vector comprises a splice donor signal sequence positioned 3′ of the first coding polynucleotide and the second vector comprises a splice acceptor signal sequence positioned 5′ of the second coding polynucleotide.

In some embodiments, the splice donor signal sequence comprises SEQ ID NO:9 and the splice acceptor signal sequence comprises SEQ ID NO:11.

In some embodiments, the first vector comprises a sequence that promotes recombination that is positioned 3′ of the splice donor signal sequence, and the second vector comprises a sequence that promotes recombination that is positioned 5′ of splice acceptor signal sequence.

In some embodiments, the sequence that promotes recombination in the first vector is a partial AP site comprising SEQ ID NO:8 and the sequence that promotes recombination in the second vector is a partial AP site comprising SEQ ID NO:10.

In some embodiments, the first coding polynucleotide encodes amino acids 1-732 of SEQ ID NOS: 2 or 4.

In some embodiments, the second coding polynucleotide encodes amino acids 733-1962 of SEQ ID NO:2.

In some embodiments, the second coding polynucleotide encodes amino acids 733-1790 of SEQ ID NO:4.

In some embodiments, the first vector comprises a first inverted terminal repeat (ITR) sequence at a position that is located 5′ of the first coding polynucleotide, and a second ITR sequence that is that is located 3′ of the first coding polynucleotide, and the second vector comprises a first ITR sequence at a position that is located 5′ of the second coding polynucleotide, and a second ITR sequence that is that is located 3′ of the second coding polynucleotide.

In some embodiments, the promoter is located at a position that is between the first ITR sequence and the first coding polynucleotide and the second ITR is located at a position that is 3′ to the splice donor site on the first vector, and the first ITR is located at a position that is 5′ to the splice acceptor site and the second ITR is located at a position that is 3′ of a poly(A) sequence on the second vector.

In some embodiments, the first ITRs in the first vector and second vector have at least 80% sequence identity to SEQ ID NO:5, and the second ITRs in the first vector and second vector have at least 80% sequence identity to SEQ ID NO:6.

In some embodiments, the second vector comprises a poly(A) sequence.

In some embodiments, the poly(A) sequence is a bovine growth hormone (bGH) poly(A) signal sequence.

In some embodiments, the poly(A) sequence is at least 80% identical to SEQ ID NO: 12.

In some embodiments, the first vector comprises SEQ ID NO:13 and the second vector comprises SEQ ID NO:14.

In some embodiments, the first vector comprises SEQ ID NO:15, and the second vector comprises SEQ ID NO:16.

In some embodiments, the first vector comprises SEQ ID NO:17 and the second vector comprises SEQ ID NO:18.

In some embodiments, the first vector comprises SEQ ID NO:19 and the second vector comprises SEQ ID NO:20.

In another aspect, the invention provides a method of treating sensorineural hearing loss in a subject, comprising administering to the subject a therapeutically effective amount of the dual vector system as provided herein.

In another aspect, the invention provides a method of treating vestibular dysfunction in a subject, comprising administering to the subject a therapeutically effective amount of the dual vector system as provided herein.

In another aspect, the invention provides a method of treating vision loss in a subject, comprising administering to the subject a therapeutically effective amount of the dual vector system as provided herein.

In some embodiments, the subject has been diagnosed as having Usher syndrome type I. In some embodiments, the dual vector system is injected into the inner ear of the subject. In some embodiments, the dual vector system is injected into the retina of the subject.

In another aspect, the invention provides a vector comprising a polynucleotide sequence of SEQ ID NO:13 or a variant thereof comprising at least 60% identity thereto.

In another aspect, the invention provides a vector comprising a polynucleotide sequence of SEQ ID NO:14 or a variant thereof comprising at least 60% identity thereto.

In another aspect, the invention provides a vector comprising a polynucleotide sequence of SEQ ID NO:15 or a variant thereof comprising at least 60% identity thereto.

In another aspect, the invention provides a vector comprising a polynucleotide sequence of SEQ ID NO:16 or a variant thereof comprising at least 60% identity thereto.

In another aspect, the invention provides a vector comprising a polynucleotide sequence of SEQ ID NO:17 or a variant thereof comprising at least 60% identity thereto.

In another aspect, the invention provides a vector comprising a polynucleotide sequence of SEQ ID NO:18 or a variant thereof comprising at least 60% identity thereto.

In another aspect, the invention provides a vector comprising a polynucleotide sequence of SEQ ID NO:19 or a variant thereof comprising at least 60% identity thereto.

In another aspect, the invention provides a vector comprising a polynucleotide sequence of SEQ ID NO:20 or a variant thereof comprising at least 60% identity thereto.

In another aspect, the invention provides a pharmaceutical composition comprising a dual vector system as provided herein.

In another aspect, the invention provides a pharmaceutical composition comprising a vector as provided herein.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. Pcdh15 knockin mice: a mammalian model to evaluate therapeutic intervention.

FIG. 2. Split gene delivery of Pcdh15.

FIG. 3. Retina and inner ear can require different transcripts of human PCDH15.

FIG. 4. Strategy shown using retina transcript of PCDH15 (aka: PCDH15-CD1). Figure shows in hybrid trans-splicing approach, the two vectors carry two separate halves of the transgene, without regions of sequence overlap; the 5′-half vector has a splice donor (SD) signal at the 3′ end of the AAV genome, while the 3′-half vector carries a splice acceptor (SA) signal at the 5′ end of the AAV genome. Concatemerization of the two vectors, reconstitutes the full-length gene. After transcription, splicing leads to the removal of the ITR (inverted terminal repeats) structure at the junction point, with restoration of the full-length, mature RNA of the transgene. In hybrid approach there is additional alkaline phosphatase (AP) region which enhance efficiency of gene expression.

FIG. 5. Dual vector constructs for retina showing hybrid-hybrid or hybrid-trans splicing strategies.

FIG. 6. Dual vector constructs for inner ear showing hybrid-hybrid or hybrid-trans splicing strategies.

FIG. 7. Dual-AAV PCDH15 vectors synthesize full length Protocadherin-15 in Pcdh15R250X MSC's In vitro. Single immunofluorescence labelling of Pcdh15+/+ and Pcdh15R250X adipose derived mesenchymal stem cells (MSCs). PCDH15 (red) and nuclei (grey). Merged images revealed synthesis of full length Protocadherin 15 with all three pairs of dual AAV-PCDH15 vectors (hybrid and trans-splicing) in Pcdh15R250X adipose derived (MSCs). Scale bar is 26 um.

FIG. 8. In vitro validation of dual AAV PCDH15 vectors by q-PCR technique. Figure: hPCDH15 expression in HEK293 cells (left graph) and Jurkat cells (right graph) with dual AAV-hybrid vector (green) and trans-splicing vector (magenta). Data reveled hPCDH15 expression in HEK293 cells is more with hybrid AAV vectors approach compared to trans-splicing approach. While hPCDH15 expression in Jurkat cells is more with trans-splicing AAV vectors approach compared to hybrid approach.

FIG. 9. Transduction efficiency of AAV2/Anc80L65 is more compared to AAV2/9 in Usher 1F patients derived fibroblasts. Transduction of AAV2/9 and AAV/Anc80L65 in Usher 1F patients derived fibroblasts. Transduction efficiency of AAV2/Anc80L65 (positive cells in green) is more compared to AAV2/9 in Usher 1F patients derived fibroblasts.

FIG. 10. Relative expression of PCDH15 mRNA in fibroblasts shows hybrid approach is better compared to trans-splicing. Transduction of AAV2/9 (Hybrid-H) AAV2/9 (trans-splicing-T) and AAV/Anc80L65-(Hybrid-H) in Usher 1F patients derived fibroblasts. Relative expression of PCDH15 mRNA in Usher 1F patients derived fibroblasts shows hybrid approach is better compared to trans-splicing approach.

FIG. 11. Experimental plan for in vivo validation of dual-AAV-Anc80-PCDH15 vector pair in the retina by subretinal retinal injections in Pcdh15R250X mice. Based on 2 month post injection ERG data we will decide to make changes in our experimental plan to see how long this rescue will persist in these mice injected with dual AAV-PCDH15 FIG. 12. ERG data shows significant rescue of ERG amplitudes at ˜1 month in Pcdh15R250X mice suggesting improved photoreceptors functional activity

FIG. 13. Exploratory behavior analysis. Open-field exploratory behavior of mice at P16 for denoted genotypes was performed which shows that injection of Dual AAV2/9-PCDH15 at P0 could rescue vestibular deficit (middle panel). Mice were put in a new cage and a camera was placed above the cage to record the movement of mouse in cage over the period of 2 minutes.

FIG. 14. Immunolabelling of PCDH15. Injection of Dual AAV2/9-PCDH15 at P0 lead to expression of PCDH15 (red) in outer and Inner hair cells at P7 as shown in comparison with Pcdh15 heterozygous and Pcdh15 homozygous uninjected mutants. PCDH15 was labelled using PB303 antibody in 1:200 dilution. Immunoreactivity of PCDH15 was visualized with fluorescently labelled secondary antibody, and F-actin was stained with phalloidin 488 nm. Images were taken using Nikon spinning disk W1 confocal microscope. Scale bar: 20 μm.

FIG. 15. Vector map encoding HBL sequence (6449 bp) human PCDH15-CD1 full sequence (Retina isoform), 5′ITR, 3′ITR, CMV Promotor, SD site +5′ partial AP site, and PCDH15 DNA seq.

FIG. 16. Vector map encoding HBR sequence (7554 bp) human PCDH15-CD1 full sequence (Retina isoform), 5′ITR, 3′ITR, SA site +3′ partial AP site, PCDH15 DNA seq, and bGH pA.

FIG. 17. Vector map encoding TSL Sequence (6157 bp) human PCDH15-CD1 full sequence (Retina isoform), 5′ITR, 3′ITR, CMV Promotor, PCDH15 DNA seq, and SD sequence.

FIG. 18. Vector map encoding TSR sequence (7263 bp) human PCDH15-CD1 full sequence (Retina isoform), 5′ITR, 3′ITR,SA site, PCDH15 DNA seq, and bGH pA.

FIG. 19. Vector map encoding HBL sequence (6449 bp) human PCDH15-CD2 full sequence (Inner ear isoform), 5′ITR, 3′ITR,CMV Promotor, SD site +5′ partial AP site, and PCDH15 DNA seq.

FIG. 20. Vector map encoding HBR sequence (7554 bp) human PCDH15-CD2 full sequence (Inner ear isoform), 5′ITR, 3′ITR, SA site +3′ partial AP site, PCDH15 DNA seq, and bGH pA.

FIG. 21. Vector map encoding TSL sequence (6157 bp) human PCDH15-CD2 full sequence (Inner ear isoform), 5′ITR, 3′ITR, CMV Promotor, PCDH15 DNA seq, and SD sequence.

FIG. 22. Vector map encoding TSR sequence (7263 bp) human PCDH15-CD2 full sequence (Inner ear isoform), 5′ITR, 3′ITR,SA site, PCDH15 DNA seq, and bGH pA.

FIG. 23. Development and in vitro validation of dual AAV based PCDH15 gene delivery strategy. (A-B) The human PCDH15 cDNA (7154 bp) exceeds the packaging capacity of single adeno-associated virus, therefore, we split the gene into two halves (left and right). Schematic representation of the two approaches, hybrid (A) and trans-splicing (B), to reconstitute full length protocadherin-15. ITR=inverted terminal repeat, CMV=cytomegalovirus (ubiquitous promoter), BGH=bovine growth hormone (polyadenylation signal). SD=splice donor site, SA=splice acceptor site, and AP=alkaline phosphatase (recombinogenic region), SP=signal peptide, EC1-EC11=E-cadherin like domains 1-11, TM=transmembrane domain, CD1=cytoplasmic domain 1. (C) In vitro validation of dual AAV, packaged in either AAV2/9 and AAV2/Anc80L65 capsids, mediated expression of full length protocadherin-15 in Pcdh15^{KI/KI} mice derived mesenchymal stem cells (MSCs). Protocadherin-15 (abbreviated as PCDH15) immunostaining (red) using an antibody against C-terminus shows reconstitution of full length protocadherin-15. DAPI stains nuclei (grey). Scale bar: 10 μm. (D) Real-time qPCR data representation of PCDH15 gene expression relative to GAPDH between the untreated (UT) and treated USH1F fibroblasts with dual AAV PCDH15 constructs AAV2/Anc80L65 hybrid (H), AAV2/9 hybrid (H), and AAV2/9 hybrid-trans splicing (T). Data represents mean±SEM of three biological replicates. Student unpaired t-test, p<0.05 (*), p<0.01 (**), or ns—not significant.

FIG. 24. Early age dual-vector subretinal delivery restores retinal function in Pcdh15^{KI/KI} mice. (A) Schematic representation of early age dual-AAV vector (AAV2/Anc80L65 PCDH15 hybrid vectors) subretinal delivery and layout for evaluation of four main tractable deficits in Pcdh15^{KI/KI} mice. (B) Mice were injected in both eyes at P18-P22 with 1 μl of single C-fragment of PCDH15 (control) or 1 μl of a 1:1 dilution of dual N- and C-fragments. Representative scotopic (dark adapted) ERG traces from littermate control [Pcdh15^{KI/+} (black) or Pcdh15^{KI/KI} (red)] and mutant [Pcdh15^{KI/KI} (magenta)] mice at denoted ages post subretinal injection revealed significant improvement of both a-wave (left panels) and b-wave (right panels) amplitudes in mutant mice that received both halves of PCDH15. (C) Quantification of photopic b-wave for the denoted mice shows PCDH15 subretinal delivery improved cone-mediated function of mutant mice. Data presented as mean±SEM. One-way ANOVA and Bonferroni post hoc test, p<0.05 (*), p<0.01 (**), p<0.001 (***), or ns—not significant.

FIG. 25. PCDH15 gene delivery restored expression of full length protocadherin-15 in the transduced photoreceptors and rescues protocadherin-15 mediated functions in the retina. (A) Dual AAV mediated PCDH15 subretinal delivery showed sustained rescue in ERG amplitudes over time in Pcdh15 mutant mice. The recovery of ERG amplitudes persisted out to 28 weeks after injection, the latest time point tested. Representative scotopic (dark adapted) ERG traces from littermate control [Pcdh15^{+/KI} (black) or Pcdh15^{KI/KI} (red)] and mutant [Pcdh15^{KI/KI} (magenta)] mice revealed sustained improvement of both a-wave and b-wave amplitudes in Pcdh15^{KI/KI} mutant mice that received both halves of PCDH15 as compared to control AAV (one part of vector pair) injected mice. (B) Immunostaining of Pcdh15 littermate controls (Pcdh15^{KI/+} or Pcdh15^{KI/KI}) and mutant (Pcdh15^{KI/KI}) mice retinae revealed expression of protocadherin-15 (brown) localized to the inner segments (IS) of photoreceptors in dual AAV PCDH15 injected mice. Notably, protocadherin-15 immunoreactivity is absent in Pcdh15^{KI/KI} retinae injected with control AAV. Zoomed-in image of the OS/IS interface. DAPI to visualize nuclei in purple. Outer segment (OS), Outer nuclear layer (ONL), outer plexiform layer (OPL), Inner nuclear layer (INL) and inner plexiform layer (IPL). Scale bar: 10 μm. (C) Quantification of indicated retinoid species shows improved quantities of 11-cis retinal oxime in in dual AAV treated Pcdh15^{KI/KI} mice (n=7) compared to age matched littermate control mice (Pcdh15^{KI/+}, n=6, and Pcdh15^{KI/KI}, n=7). (D) Dual AAV mediated PCDH15 gene delivery improved levels of visual cycle enzymes in Pcdh15^{KI/KI} mice. Data presented as mean±SEM. Each data point represents an individual mouse. Student's unpaired t-test. p<0.05 (*), p<0.01 (**), or ns—not significant.

FIG. 26. PCDH15 gene delivery partially improved translocation of arrestin and transducin in response to light signal. Representative confocal micrographs of light-adapted retinae showed partially improved localization of phototransduction cascade proteins, transducin and arrestin (A), to both the inner segment (IS) and outer segment (OS) in mutant mice treated with dual AAV (bottom panels). In control mice, transducin is correctly localized to the IS and arrestin is to the OS (top panels). Scale bar: 10 μm. (B) Schematics of the localization of transducin and arrestin in control, Pcdh15^{KI} injected mice are shown. Quantified expression showed partial rescue of both arrestin and transducin in IS/OS respectively. Students unpaired t-test. p<0.05 (*), p<0.01 (**), p<0.001 (***), p<0.0001 (****), or ns—not significant.

FIG. 27. Patient-derived Induced Pluripotent Stem Cell (iPSC) Expansion and Retinal Organoid (RO) Differentiation. (A) Schematic representation of tissue culture plan for iPSC expansion and RO differentiation. KO Serum=Knockout Serum Replacement. (B) iPSCs derived from an unaffected control (Con), isogenic control (IsoCon) and an Usher 1F patient (Ush1F) were differentiated into ROs. Temporal images of each cell line were taken at denoted timepoints. Phase bright areas (red arrows) indicate neuro epithelium. Scale bar=500 μm. (C) Gene expression analysis of developing ROs indicate downregulation of pluripotency markers OCT4, SOX2 and NANOG in Con, IsoCon and Ush1F lines upon differentiation at day 30, 45 and 60. Data is expressed as log (2) fold change relative to gene expression in iPSCs. All data was normalized to house-keeping genes GAPDH and RPL13A; technical triplicates. Data presented as mean±SEM. (D) ROS expression of retinal progenitor and photoreceptor-specific genes (PAX6, RAX, NRL, RHO, and ARR3) relative to adult human post-mortem retina. (E) Immunohistochemistry showed the presence of the developing connecting cilium at the apical edge of the developing outer nuclear layer by ARL13B immunostaining, indicating photoreceptors were beginning to develop morphologically mature characteristics. Scale bar=10 μm. (F) Comparison of endogenous PCDH15 expression in ROs, the iPSCs from which they were derived, and native human retina from post-mortem adult samples. Data is normalized to housekeeping genes GAPDH. Relative PCDH15 levels were significantly different. Students unpaired t-test. p<0.0001 (****) between human retina and all ROs and iPSC samples where n>1. Data presented as mean±SEM.

FIG. 28. Relative serotype efficacy in Retinal Organoids (ROs) and evaluation of dual AAV PCDH15 constructs in patient-derived ROs. (A) Single AAV transduction efficacy in ROs showed higher transduction with AAV2/Anc80L65-CMV.GFP as compared to AAV2/9-CMV.GFP for ROs transduced at day 30 and assessed at denoted timepoints post-transduction. Scale bar=500 μm. (B) Co-transduction of ROs with AAVs carrying either GFP or mCherry at day 45 post-differentiation and analysis after 14 days post-transduction showed comparable transduction for both AAV2/Anc80L65 and AAV2/9 capsids. Scale bars=1.0 mm. (C) CRX expression relative to human retina in un-transduced (UT), AAV2/9, AAV2/Anc80L65 carrying fluorescent markers (GFP or mCherry) and IsoCon ROs at day 90 and 120 post-differentiation showed positive correlation with PCDH15. Statistical significance p<0.05 (*) between IsoCon and untransduced (UT), AAV2/9 and AAV2/Anc80L65 co-transduced groups. (D) PCDH15 and CRX transcripts expression in ROs transduced with dual AAV PCDH15 constructs. PCDH15 transcript expression relative to human retina in untreated USH1F (UT), AAV2/9 treated USH1F (AAV9), AAV2/Anc80L65 treated USH1F (Anc80) and isogenic control (IsoCon) retinal organoids at day 90 and 120 post-differentiation. All ROs were treated at day 45 post-differentiation with 1×10¹⁰ viral particles per vector, per organoid. Statistical significance p<0.05 (*) between day 90 and 120. Data presented as mean±S.E.M. (E) Linear correlation (R²=0.8629) found for PCDH15 and CRX transcript expression in IsoCon relative to UT, while nonlinear relationship was observed (R²=0.0281) for AAV2/Anc80L65 PCDH15 and CRX transcript expression relative to UT. However, linear correlation was restored (R²=0.8370) for AAV2/9 PCDH15 and CRX transcript expression relative to UT. Treatment groups at each time point consisted of three biological replicates. All data was normalized to endogenous housekeeper GAPDH.

FIG. 29. Later age dual-vector subretinal delivery did not rescue the retinal function in Pcdh15^{K/KI} mice. Mice injected in both eyes at P30 with 1 μl of single right end vector of PCDH15 packaged into AAV2/Anc80/L65 (control AAV) or 1 μl of a 1:1 dilution of dual left and right AAV2/Anc80L65 vectors of hybrid approach. Retinal function of these mice was evaluated via ERGs 1- and 2-months post injections and compared to Pcdh15^{KI/+} control littermates. We observed no statistically significant improvement in the ERG amplitudes for both scotopic (dark adapted) a-wave and b-wave in Pcdh15^{KI/KI} mutant mice (n=5) that received dual viruses as compared to control AAV (n=4). Data presented as mean±SEM. One-way ANOVA and Bonferroni post hoc test, p<0.01 (**), or ns—not significant.

FIG. 30. Subretinal injection of dual AAV vectors showed no overt toxicity in mice post injection. (A) Pcdh15^{KI/+} mice that received subretinal injection of dual AAV2/Anc80L65 vectors (n=15) had no discernable differences in ERG amplitudes for both scotopic a-wave and b-wave when compared with Pcdh15^{KI/+} uninjected mice (n=7). (B) Quantification of photopic (light adapted) b-wave for the denoted mice showed no changes in cone-mediated function of Pcdh15^{KI/+} mice (injected vs uninjected). Data presented as mean±SEM. One-way ANOVA and Bonferroni post hoc test ns—not significant. (C) AAV mediated subretinal delivery of PCDH15 showed no overt retinal degeneration in mice of denoted genotype as measured by non-invasive optical coherence tomography (OCT). Quantification of outer nuclear layer (ONL) thickness showed no differences across mice of denoted genotype and treatment. Injected mice were assessed 2-3 months post injection. Data presented as mean±SEM Student's unpaired t-test, ns-non-significant.

FIG. 31. Dual AAV mediated PCDH15 gene delivery improved levels of visual cycle enzymes in Pcdh15^{KI/KI} mice. Representative immunoblot of key proteins involved in the visual retinoid cycle shows improved quantities of RPE65 and CRALBP in dual AAV treated Pcdh15^{KI/KI} mice.

FIG. 32. Gene expression analysis of developing retinal organoids (ROs). Fold change in SOX7 (endoderm), DCX (ectoderm) and TBXT (mesoderm) at day 30 and 45 ROs, relative to iPSCs. Data was normalized to house-keeping genes GAPDH. Each sample was run in triplicate. Data presented as mean±SEM.

FIG. 33. Dual AAV capsids co-transduction in retinal organoids (ROs). USH1F ROs treated with either AAV2/9-CMV.GFP and AAV2/9-CMV.mCHERRY, or AAV2/Anc80L65-CMV.GFP and AAV2/Anc80L65-CMV.mCHERRY at day 45 post-differentiation and visualized at 14 days post-transduction. Immunostaining showed expression of photoreceptor marker (CRX) in retinal organoids untreated and transduced with AAV2/Anc80L65 and AAV2/9 vectors. Scale bars represent 125 μm.

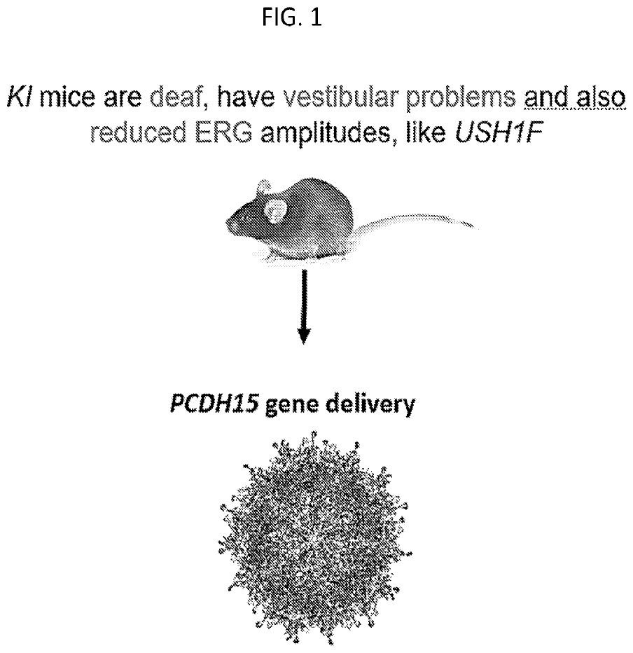

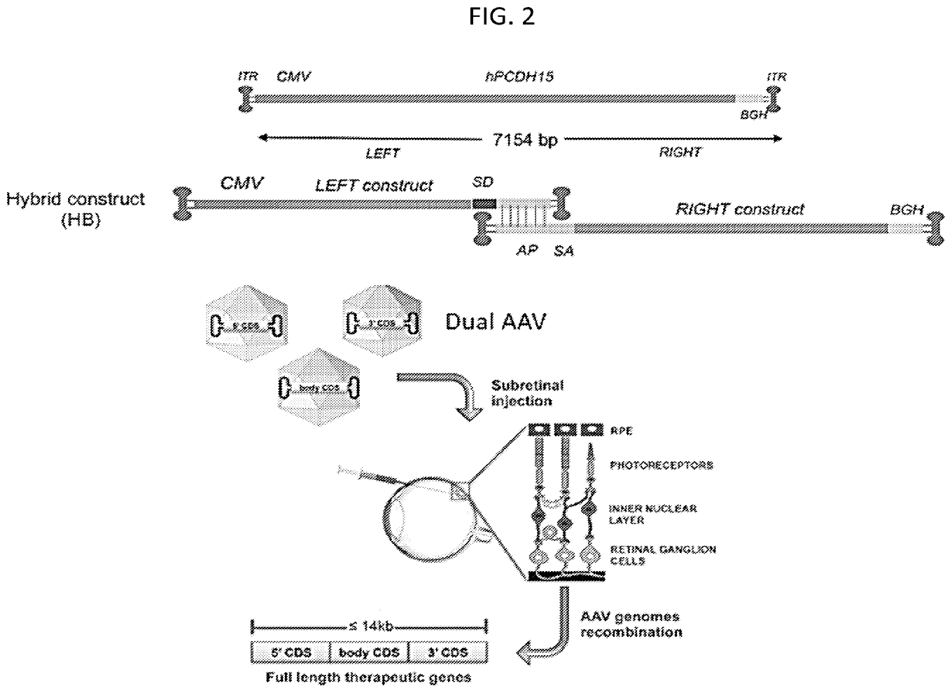

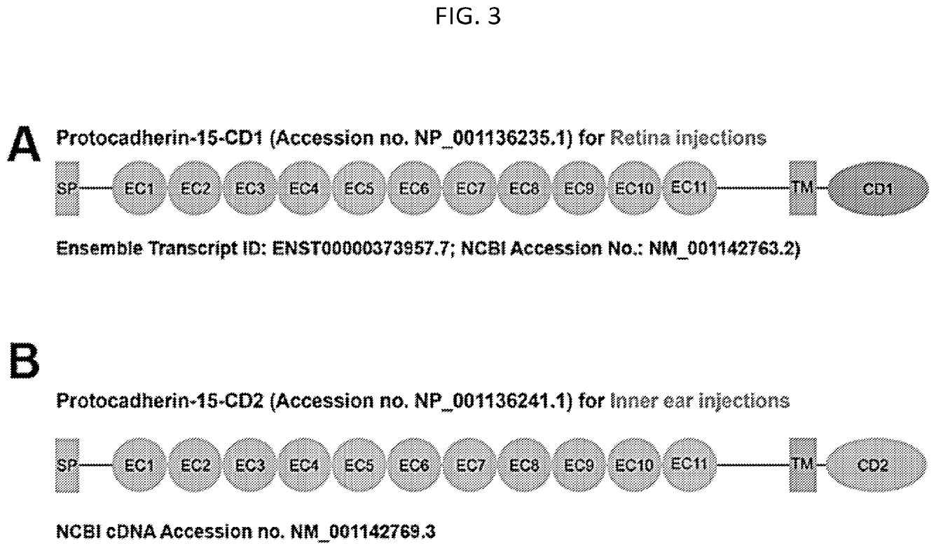

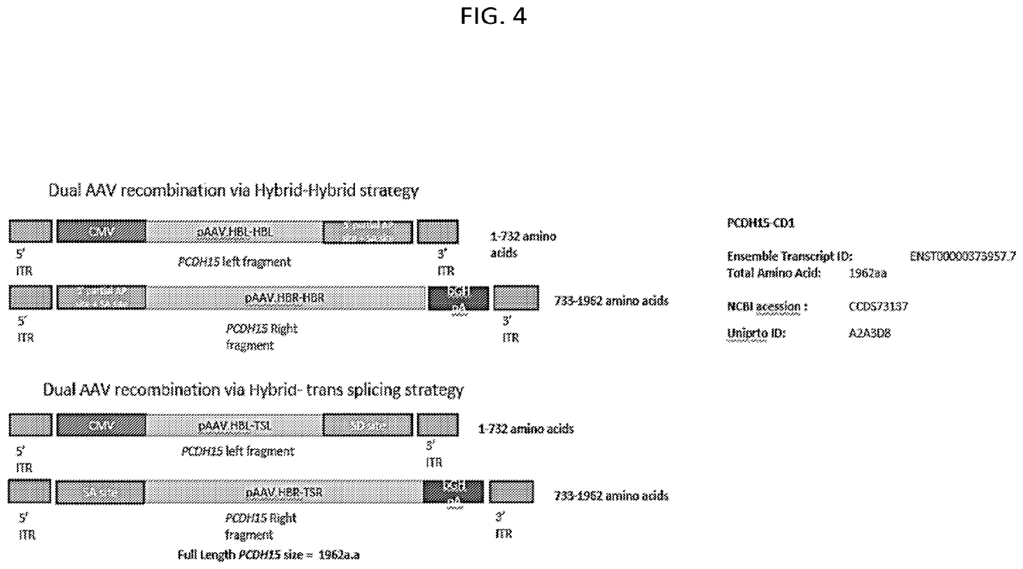

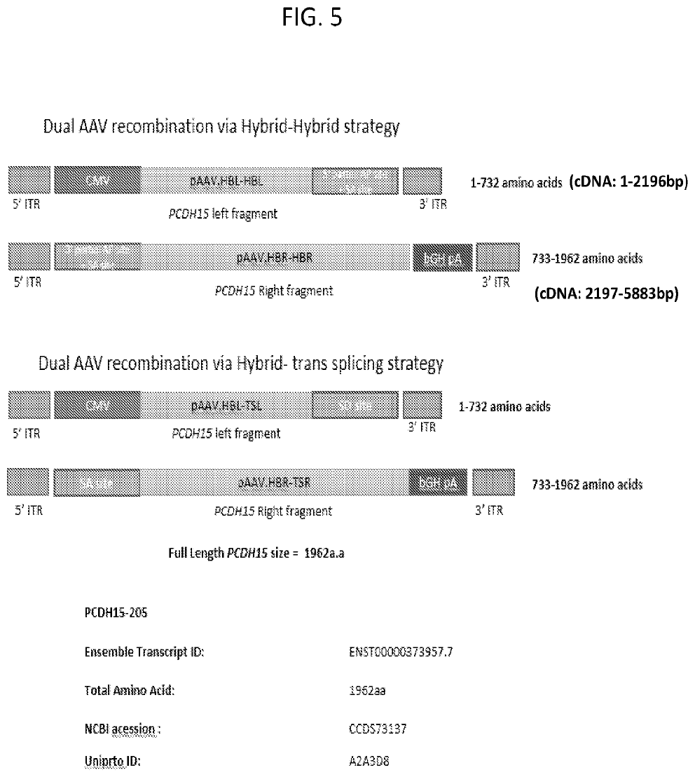

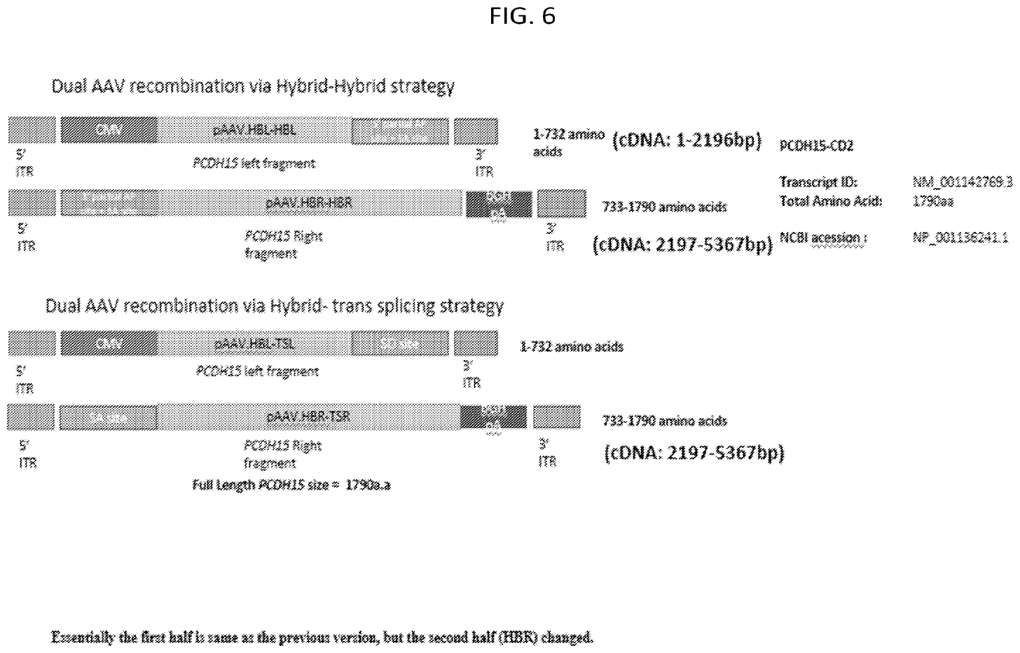

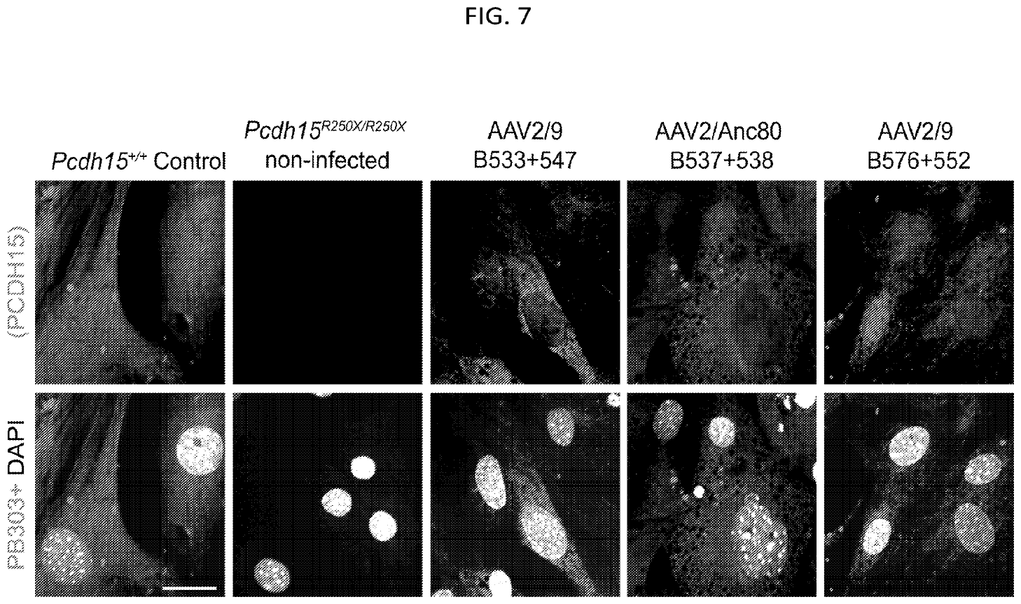

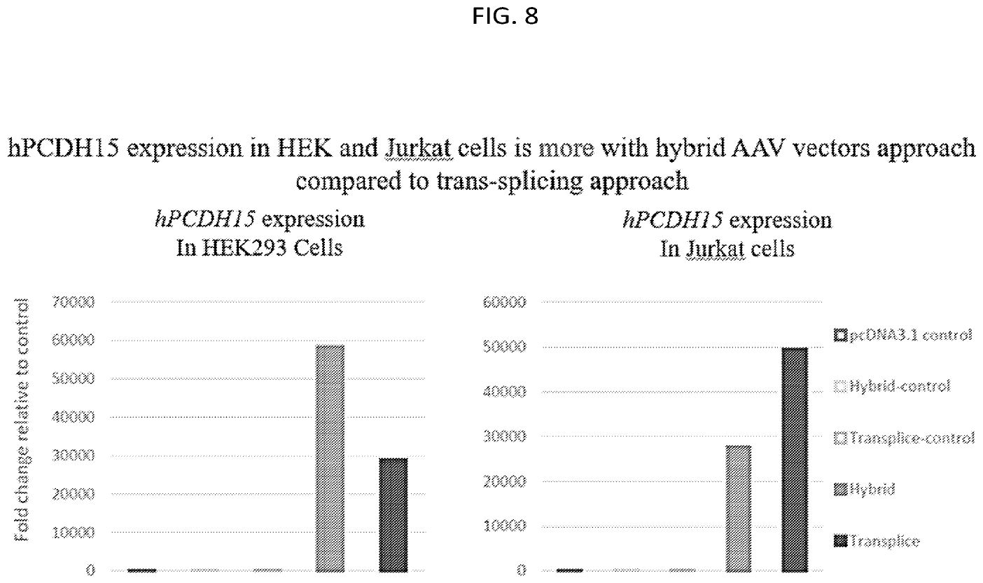

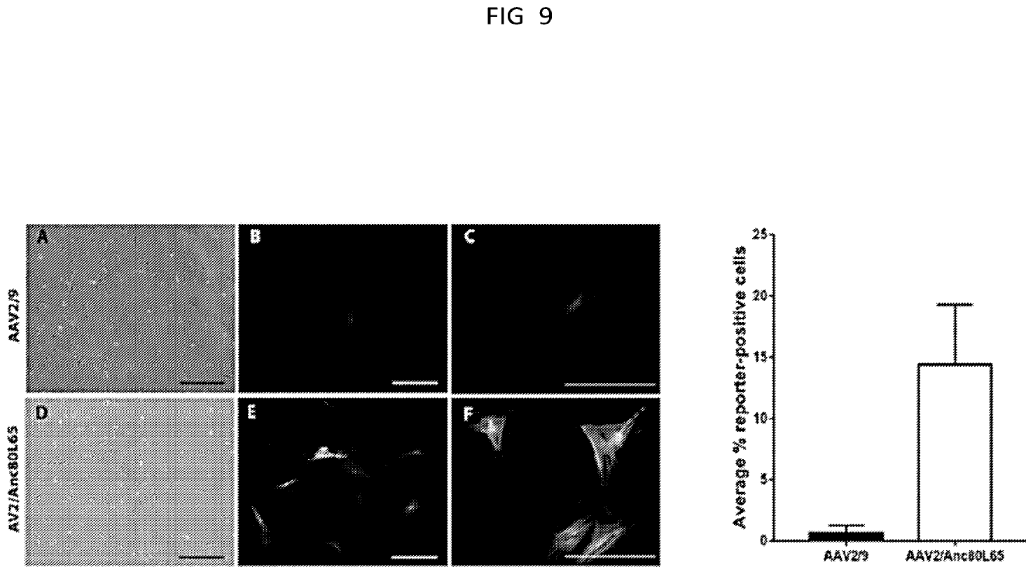

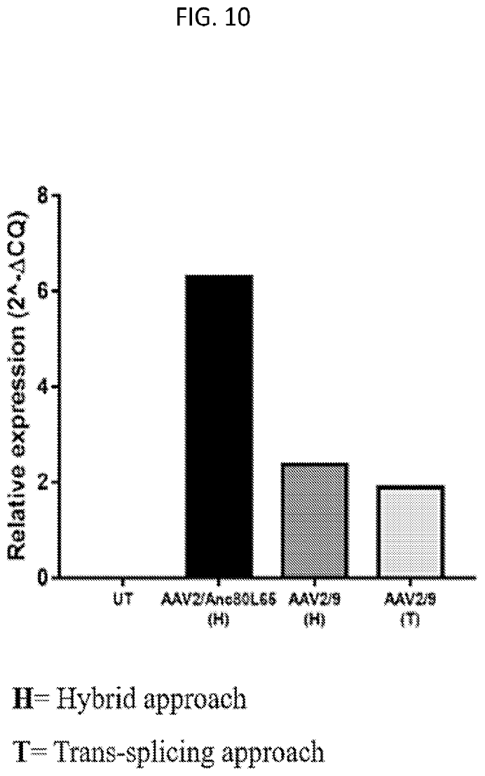

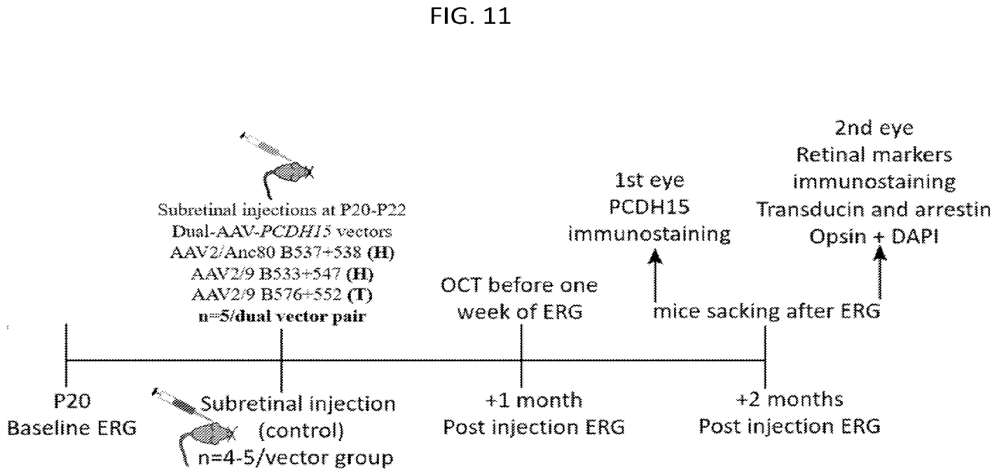

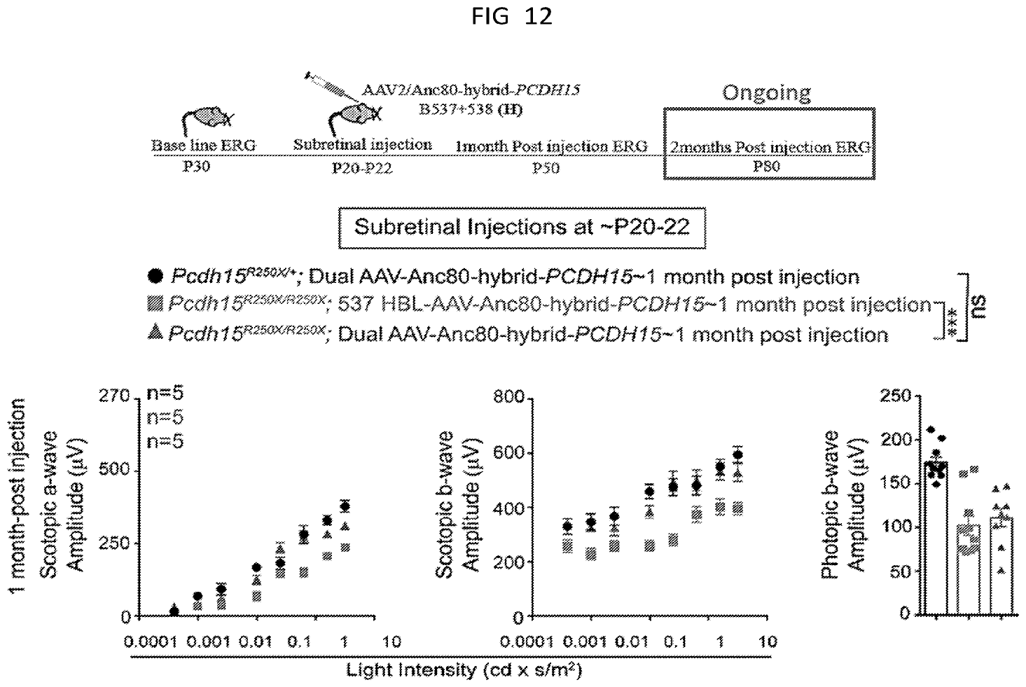

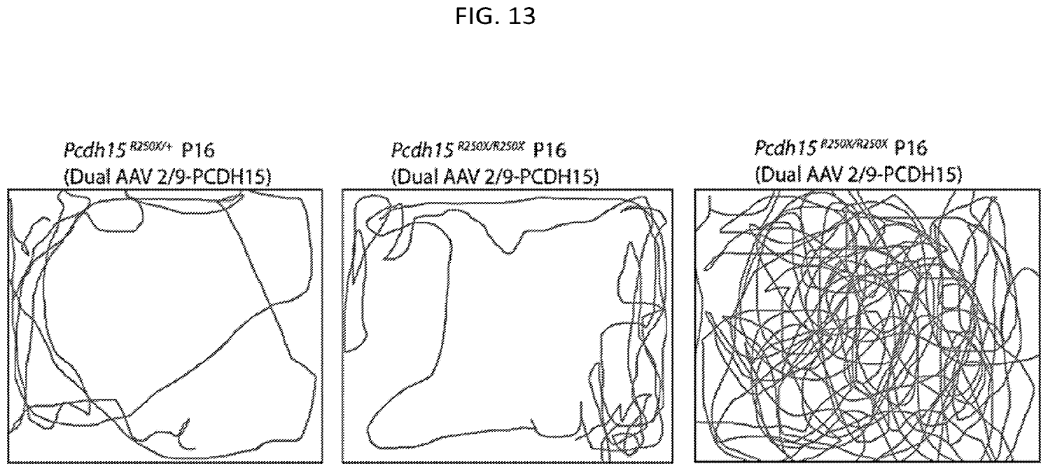

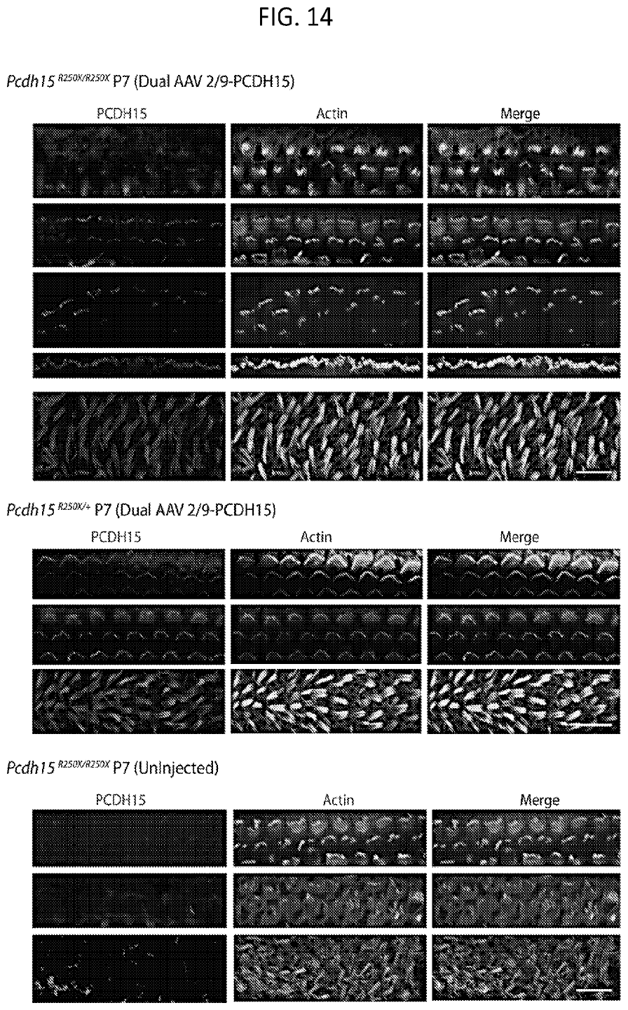

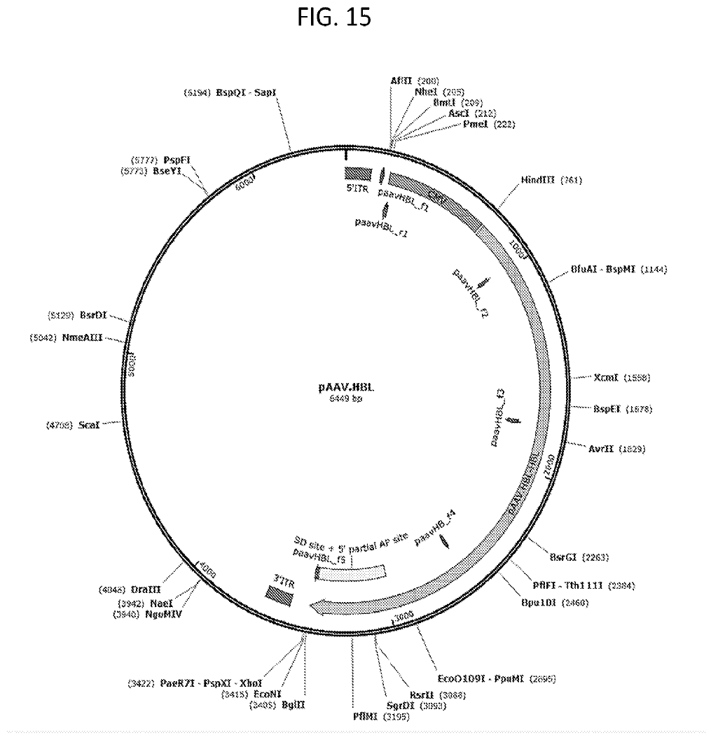

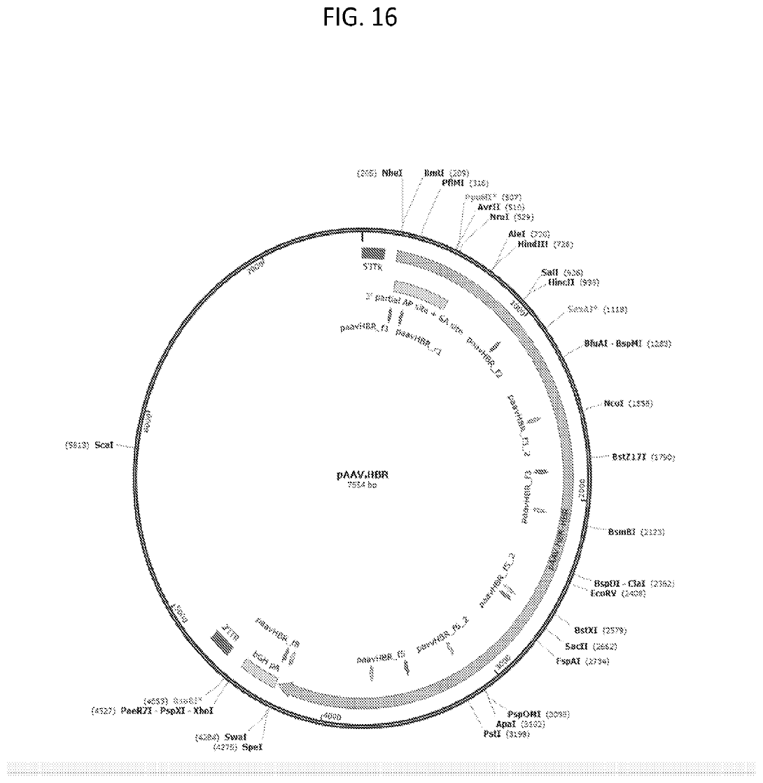

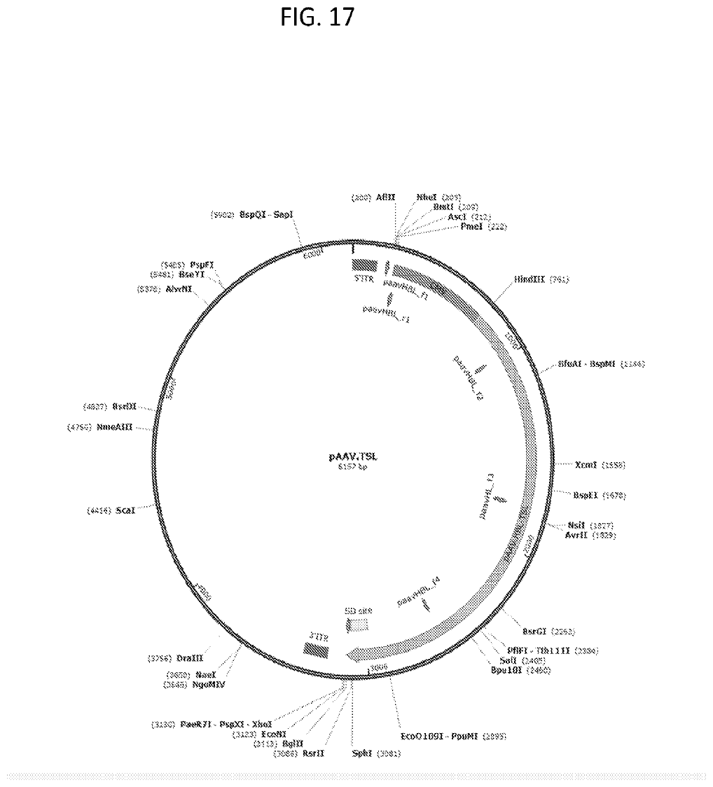

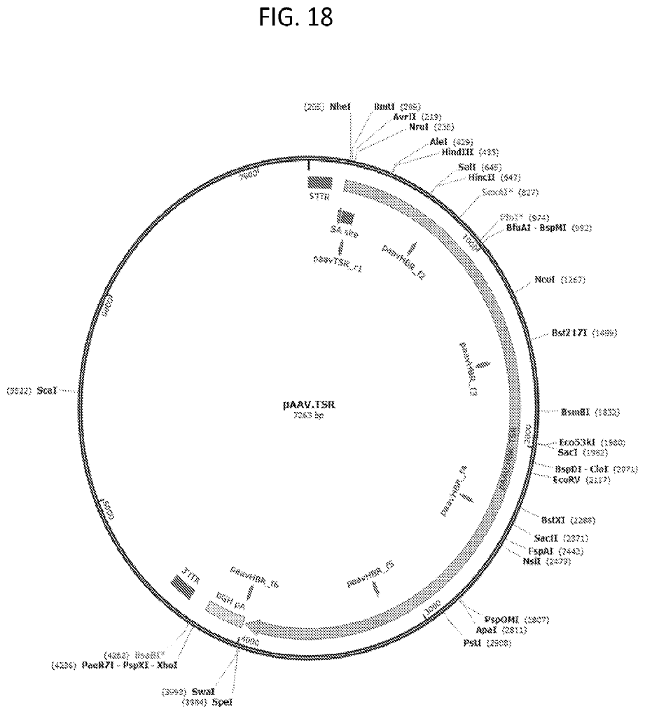

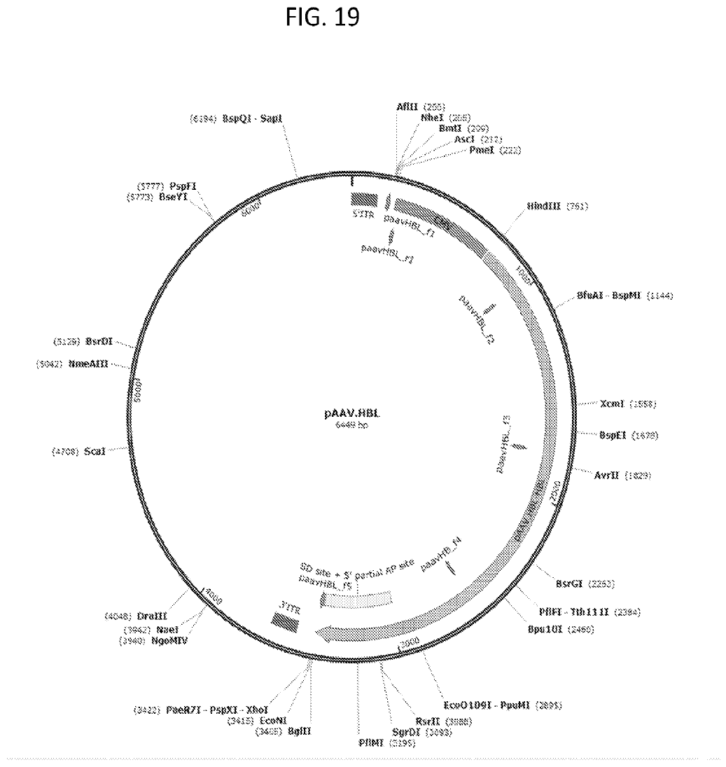

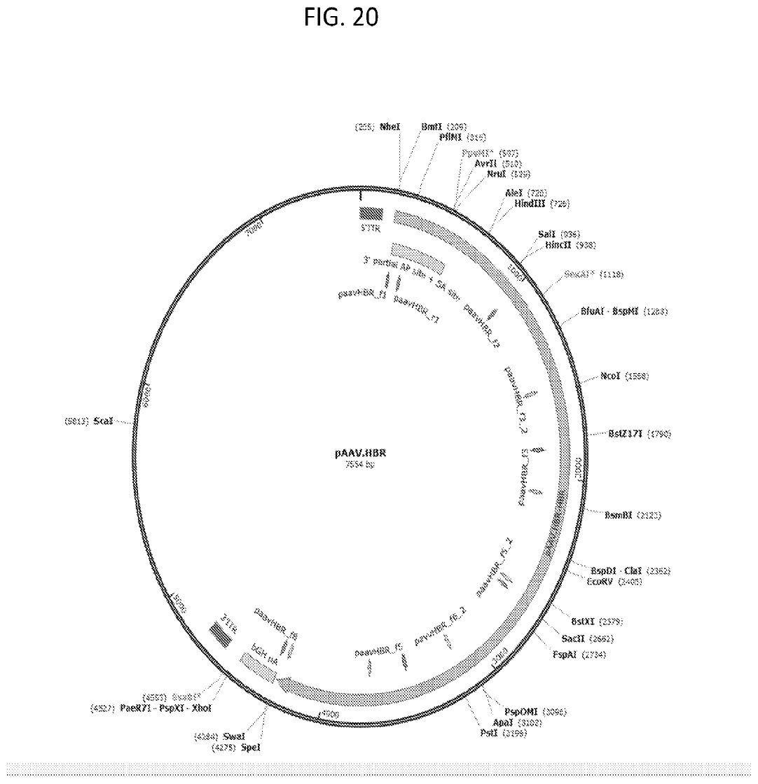

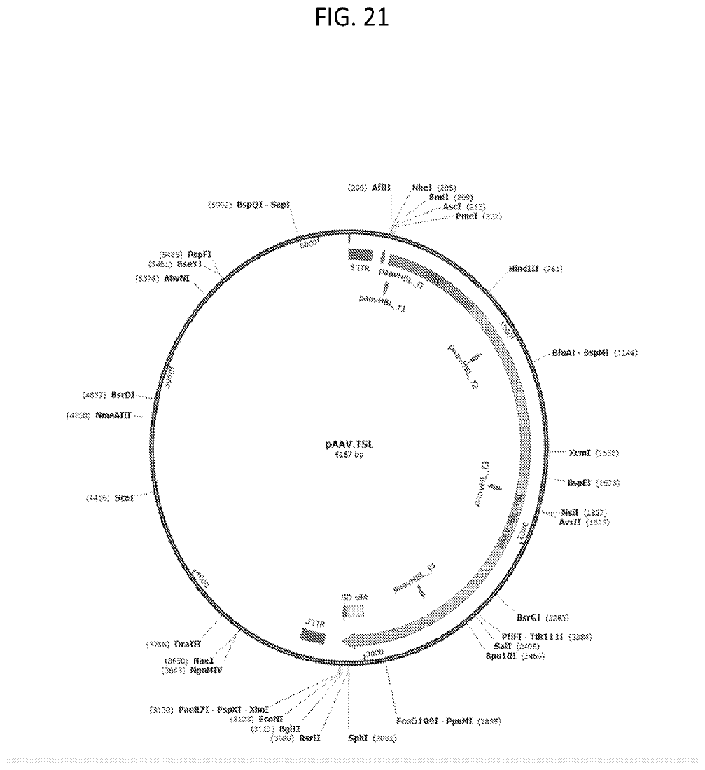

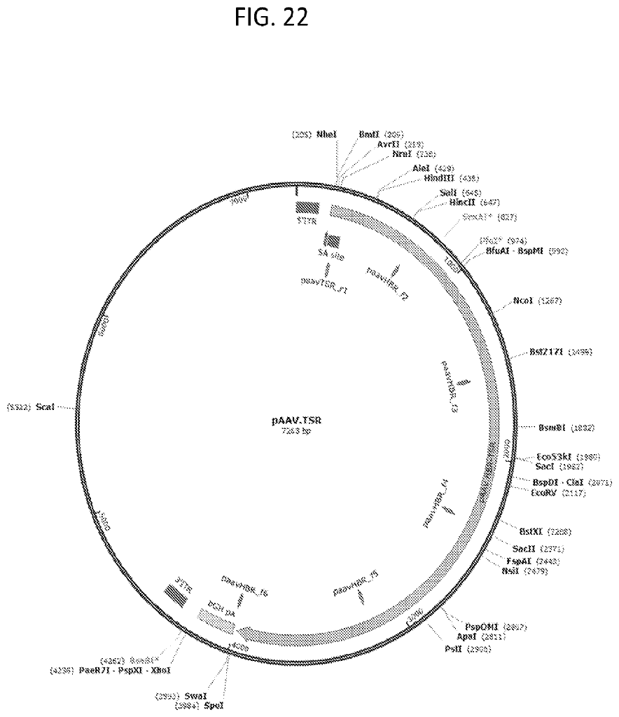

### DETAILED DESCRIPTION OF THE INVENTION

The present invention is based, in part, on the surprising discovery that various Protocadherin-15 isoforms when delivered via a dual vector system can treat sensorineural hearing loss, vestibular dysfunction and/or vision loss.

Reference will now be made in detail to embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Furthermore, where the one or more embodiments uses the term “comprising,” those skilled in the art would understand that, in some specific instances, the embodiment or embodiments can be alternatively described using the language “consisting essentially of” and/or “consisting of.” As used herein, the term “about” means at most plus or minus 10% of the numerical value of the number with which it is being used.

It is contemplated that any method or composition described herein can be implemented with respect to any other method or composition described herein.

One skilled in the art may refer to general reference texts for detailed descriptions of known techniques discussed herein or equivalent techniques. These texts include Current Protocols in Molecular Biology (Ausubel et. al., eds. John Wiley & Sons, N.Y. and supplements thereto), Current Protocols in Immunology (Coligan et al., eds., John Wiley St Sons, N.Y. and supplements thereto), Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

The compositions and methods described herein can be used to treat sensorineural hearing loss, vestibular dysfunction and/or vision loss in a subject by administering a first nucleic acid vector containing a polynucleotide encoding an N-terminal portion of an Pcdh15 or variant thereof and a second nucleic acid vector containing a polynucleotide encoding a C-terminal portion of an Pcdh15 or variant thereof.

In some embodiments, the method comprise administering a first nucleic acid vector (e.g., an AAV vector) containing a promoter and a polynucleotide encoding an N-terminal portion of a Protocadherin-15 protein (e.g., a wild-type (WT) human Pcdh15) or a variant thereof and a second nucleic acid vector (e.g., an AAV vector) containing a polynucleotide encoding a C-terminal portion of an Protocadherin-15 or a variant thereof and a polyadenylation (poly(A)) sequence.

In some embodiments, these compositions and methods can be used to treat subjects having one or more mutations in the Pcdh15 gene, e.g., an Pcdh15 mutation that reduces Pcdh15 expression, reduces Pcdh15 function, or is associated with hearing loss, vision loss or vestibular dysfunction (e.g., a frameshift mutation, a nonsense mutation, a deletion, or a missense substitution). When the first and second nucleic acid vectors are administered in a composition, the polynucleotides encoding the N-terminal and C-terminal portions of Pcdh15 or variant thereof can combine within a cell (e.g., a human cell, e.g., an inner ear or retinal cell) to form a single nucleic acid molecule that contains the full-length Pcdh15 coding sequence (e.g., through homologous recombination and/or splicing).

In some embodiments, the invention provides a method of treating sensorineural hearing loss in a subject, comprising administering to the subject a therapeutically effective amount of a dual vector system as provided herein.

In some embodiments, the invention provides a method of treating vestibular dysfunction in a subject, comprising administering to the subject a therapeutically effective amount of a dual vector system as provided herein.

In some embodiments, the invention provides a method of treating vision loss in a subject, comprising administering to the subject a therapeutically effective amount of a dual vector system as provided herein.

In some embodiments, the subject has been diagnosed as having Usher syndrome type I. In some embodiments, the dual vector system is injected into the inner ear of the subject. In some embodiments, the dual vector system is injected into the retina of the subject.

In accordance with the invention, a “therapeutically effective amount” or “effective amount” is administered to the subject. As used herein a “therapeutically effective amount” or “effective amount” is an amount sufficient to decrease, suppress, or ameliorate one or more symptoms associated with the disease or condition.

As used herein, “treat” and all its forms and tenses (including, for example, treating, treated, and treatment) refers to therapeutic and prophylactic treatment. In certain aspects of the invention, those in need of treatment include those already with a pathological disease or condition of the invention (including, for example, hearing loss), in which case treating refers to administering to a subject (including, for example, a human or other mammal in need of treatment) a therapeutically effective amount of a composition so that the subject has an improvement in a sign or symptom of a pathological condition of the invention. The improvement may be any observable or measurable improvement. Thus, one of skill in the art realizes that a treatment may improve the patient's condition, but may not be a complete cure of the disease or pathological condition.

In some embodiments, the subject can be a mammal. In some embodiments, the mammal can be a human. In some embodiments of the above-described methods, administration of the dual vector system to the subject is targeted to a specific type of cell. In some embodiments, the dual vector system can be administered systemically to the subject. In other embodiments, the pharmaceutical composition can be administered directly to at least one tissue of the subject, such as the eye, retinal tissue, or inner ear tissue.

In some embodiments, the invention provides a dual vector system for expressing a Protocadherin-15 protein or variant thereof in a subject, wherein the Protocadherin-15 protein comprises SEQ ID NO:2, wherein the dual vector system comprises
i) a first vector comprising a first coding polynucleotide that encodes an N-terminal portion of the Protocadherin-15 protein or variant thereof; and
ii) a second vector comprising a second coding polynucleotide that encodes a C-terminal portion of the Protocadherin-15 protein or variant thereof,
wherein the first coding polynucleotide and the second coding polynucleotide that encode the Protocadherin-15 protein or variant thereof do not overlap.

In some embodiments, the invention provides a dual vector system for expressing a Protocadherin-15 protein or variant thereof in a subject, wherein the Protocadherin-15 protein comprises SEQ ID NO:4, wherein the dual vector system comprises
i) a first vector comprising a first coding polynucleotide that encodes an N-terminal portion of the Protocadherin-15 protein; and
ii) a second vector comprising a second coding polynucleotide that encodes a C-terminal portion of the Protocadherin-15 protein,
wherein the first coding polynucleotide and the second coding polynucleotide that encode the Protocadherin-15 protein or variant thereof do not overlap.

The terms “polypeptide,” “peptide,” and “protein” are used interchangeably herein to refer to a polymer of amino acid residues. The terms apply to naturally occurring amino acid polymers as well as amino acid polymers in which one or more amino acid residues is a non-naturally occurring amino acid, for example, an amino acid analog. As used herein, the terms encompass amino acid chains of any length, including full length proteins, wherein the amino acid residues are linked by covalent peptide bonds.

In some embodiments, the Protocadherin-15 protein is an isoform having the amino acid sequence found in accession number NP_001136235.1 and has the amino acid sequence comprising SEQ ID NO:2. In some embodiments, this isoform has a nucleotide sequence comprising SEQ ID NO:1. In some embodiments, this isoform or variants thereof are suitable for treating vision loss in a subject.

In some embodiments, the Protocadherin-15 protein is an isoform having the amino acid sequence found in accession number NP_001136241.1 and has the amino acid sequence comprising SEQ ID NO:4. In some embodiments, this isoform has a nucleotide sequence comprising SEQ ID NO:3. In some embodiments, this isoform or variants thereof are suitable for treating hearing loss and/or vestibular dysfunction in a subject.

TABLE 1
Protocadherin-15 nucleotide and amino acid sequences

| SEQ ID | |
|---|---|
| NO: | Sequence |
| 1 | atgtttcgacagttttatctctggacatgtttagcttcagggatcatcctgggctctctctttgaaatctgcttgg |
| | gccagtatgatgatgactggcaatatgaggattgcaaactagctaggggaggaccaccagctaccatag |
| | ttgctattgatgaagaaagtcggaatggtacaattctggtggacaacatgctgatcaaagggactgctgga |
| | ggaccagaccccaccatagaactttctttaaaggataatgtggattactgggtgttgatggatcctgttaag |
| | caaatgcttttcctgaacagcaccggaagagttctggatagagatccaccgatgaacatacactccattgt |
| | ggtgcaggtccagtgcatcaacaaaaaagtgggcactattatctaccatgaagtgcgaatagtggtgaga |
| | gacaggaatgacaactcacccactttcaagcatgaaagctactatgccacagtgaatgagctcactccag |
| | ttggtaccacaatattcacaggattttcaggagacaatggagctacagatatagatgatggaccaaatgga |
| | cagatagagtatgttattcagtataatccagatgatccgacatccaatgacacctttgaaattcccctaatgtt |
| | gactggaaatatagtgttaaggaagaggctcaactatgaagataagactcgctactttgtcataatccaag |
| | ctaatgaccgtgcccaaaatctgaatgagaggcgaaccaccaccaccactctcacagtggatgttctgga |
| | tggagatgacttgggtccaatgtttcttccttgtgtccttgtgccaaacactcgtgattgccgtccactcactt |
| | atcaagctgccatacctgagttgagaactccggaagaactgaaccccattattgttacgccaccaatccaa |
| | gccattgatcaggaccggaatattcaaccgccatcagataggccaggaatcctctattccatccttgttgg |
| | gactcctgaggattacccacgatttttccatatgcatcctaggacagcagaacttagtctcctggagccagt |
| | aaacagagactttcaccagaaatttgatttggttattaaggctgaacaagacaatggtcatcctcttcctgcc |
| | tttgccggtctacacattgaaatactggatgaaaacaatcaaagtccatattttacaatgcccagttatcaag |
| | gctatatcctggaatctgccccagtgggagcaaccatttcggacagtctcaatttgacttcacctttaagaat |
| | agtagctctggacaaggacatagaagatacaaaagacccagagcttcacctttttctgaatgactacacct |
| | cagtcttcaccgtcacacagactggtattactcgctacctcaccttacttcaaccagtggacagggaagaa |
| | cagcaaacttacaccttttcgataacagcatttgatggtgtacaagaaagtgagccagtcatcgtcaatattc |
| | aagtgatggatgcaaatgataacacgccaaccttccctgaaatatcctatgatgtgtatgtttatacagacat |
| | gagacctggggacagtgtcatacagctcactgcagtcgacgcagacgaagggtcaaatggggagatca |
| | catatgaaatccttgttggggctcagggagacttcatcatcaataaaacaacagggcttatcaccatcgctc |
| | caggggggaaatgatagtcgggcggacttacgcactcacggtccaagcagcggataatgctcctcctg |
| | cagagcgaaggaactccatctgcactgtgtatattgaagtgcttccaccaaataatcaaagccctcctcgc |
| | ttcccacagctgatgtatagccttgaaattagtgaagccatgagggttggtgctgttttattaaatctacagg |
| | caactgatcgagagggagactcaataacatatgccattgagaatggagatcctcagagagtttttaatcttt |
| | cagaaaccacggggattctaaccttagggaaagcactggacagggaaagcactgatcgctacattctga |
| | tcatcacagcttcagatggcaggccagatgggacctcaactgccacagtaaacatagtggtgacagatgt |
| | caatgacaatgctccagtgtttgatccttatctgccaagaaatttatctgtggtggaagaagaagccaatgc |
| | ctttgtgggtcaagtaaaagcaacagaccctgatgctggaataaatggtcaagtgcactacagtttgggta |
| | actttaataatctttttcgtatcacatccaatgggagcatttacacagcagtgaagcttaacagagaagtcag |
| | ggactactatgaacttgttgttgtggcaacagatggagcagtacaccctcgtcattcaactctaaccttggc |
| | catcaaggttttggacattgatgataacagtcctgtgttcaccaattcaacatacactgtccttgttgaagaga |
| | atttgccagctgggactaccatccttcaaatagaggccaaagatgtcgaccttggagcaaatgtgtcttac |
| | cggataagaagcccagaagtgaagcacttttttgcactacatccatttacaggagaactatcgcttttaagg |
| | agtttagattatgaggcatttccagaccaagaagcaagtatcacttttctggtagaggcctttgatatttatgg |
| | aacaatgccacctggtattgctactgtcacagtgattgtaaaggatatgaatgattatcctcctgtctttagta |
| | aacgaatatacaaagggatggtggctccggatgcagtcaagggtacacctatcacaacagtttatgctga |
| | agatgcagaccctcctggattacctgcaagtcgtgtgaggtatagagtagatgatgtacagtttccttaccc |
| | tgccagtatttttgaagtggaagaagattctggaagagtaataacacgagtcaatcttaatgaagaacctac |
| | aacaatttttaagttggtggtggttgcttttgatgatggggagcctgtgatgtccagcagtgccacagtgaa |
| | gattcttgtcttacatcctggtgagatcccacgcttcacacaggaggaatatagacctcctccagtaagtga |
| | acttgccaccaaagggaccatggttggtgtaatttctgctgctgccattaatcaaagtattgtgtactccattg |
| | tttcaggaaatgaagaagatacatttggaattaataacatcacaggtgttatctatgtgaatggacctctgga |
| | ttatgagaccaggacaagctatgtacttcgagtccaagctgattccctggaagtggtccttgccaatctccg |
| | agttccttcaaaaagcaatacagctaaagtatacattgagattcaggatgaaaataatcatcccccagtgttt |
| | cagaaaaaattctacatcggaggtgtatctgaagatgcaagaatgtttacttctgtactcagagtgaaggct |
| | actgataaagatactggcaattatagtgtcatggcctacagactcataataccaccaattaaagagggaaa |
| | agaaggatttgtagtggaaacatatacagggcttatcaaaactgctatgctcttccataatatgaggagatc |
| | ctacttcaagtttcaagttattgcaactgacgactatgggaagggactgagcggcaaagccgatgtactcg |
| | tctccgtggtcaatcagctggatatgcaagtcattgtttccaatgtgcctcctactctagtggaaaaaaagat |
| | agaagatcttacagagatcttggatcgctatgttcaggaacaaattcctggtgccaaggtcgtagtggagt |
| | ccattggagctcgccggcatggagatgccttttccctagaagattacaccaaatgtgacttgactgtctatg |
| | caattgacccccaaaccaacagagccatcgatagaaatgagctttttaaatttttggatggcaaactacttg |
| | atatcaataaagactttcagccgtattatggggaaggaggacgcattctggagatccggactccagaggc |
| | agtgaccagcattaaaaagagaggagaaagtctaggatacacagaaggggccttgttggctctggcctt |
| | catcatcatcctctgctgcattcctgccatcttggtggttttggtcagctacagacagtttaaagtacgtcaag |
| | ctgagtgtacaaagactgcacgaattcaggccgcattacccgcggctaaaccagcagtgccggctcctg |
| | caccagtggcagcgcccccgccgccgccgccgcctccgccaggtgcgcatctctatgaagaacttgga |
| | gacagctcaatgcataatcttttccttctctaccattttcaacaaagcaggggaaataactcagtctcagaag |
| | acaggaaacatcaacaagttgtgatgcccttttcttccaatactattgaggctcacaagtcagctcatgtaga |
| | cggatcacttaagagcaacaaactgaagtctgcaagaaaattcacatttctatctgatgaggatgacttaag |
| | tgcccataatcccctttataaggaaaacataagtcaagtatcaacaaattcagacatttcacagagaacaga |
| | ttttgtagacccattttcacccaaaatacaagccaagagtaagtctctgaggggcccaagagaaaagattc |
| | agaggctgtggagtcagtcagtcagcttacccaggaggctgatgaggaaagttccaaatagaccagag |
| | atcatagatctgcagcagtggcaaggcaccaggcagaaagctgaaaatgaaaacactggaatctgtaca |
| | aacaaaagaggtagcagcaatccattgcttacaactgaagaggcaaatttgacagagaaagaggaaata |
| | aggcaaggtgaaacactgatgatagaaggaacagaacagttgaaatctctctcttcagactcttcattttgc |
| | tttcccaggcctcacttctcattctccactttgccaactgtttcaagaactgtggaactcaaatcagaacctaa |
| | tgtcatcagttctcctgctgagtgttccttggaactttctccttcaaggccttgtgttttacattcttcactctcta |
| | ggagagagacacctatttgtatgttacctattgaaaccgaaagaaatatttttgaaaattttgcccatccacc |
| | aaacatctctccttctgcctgtccccttccccctcctcctcctatttctcctccttctcctcctcctgctcctgctc |
| | ctcttgctcctcctcctgacatttctcctttttctcttttttgtcctcctccctctcctccttctatccctcttcctctt |
| | cctcctcctacattttttccactttccgtttcaacgtctggtcccccaacaccacctcttctacctccatttccaac |
| | tcctcttcctccaccacctccttctattccttgccctccacctccttcagcttcatttctgtccacagagtgtgtc |
| | tgtataacaggtgttaaatgcacgaccaacttgatgcctgccgagaaaattaagtcctctatgacacagcta |
| | tcaacaacgacagtgtgtaaaacagaccctcagagagaaccaaaaggcatcctcagacacgttaaaaac |
| | ttagcagaacttgaaaaatcagtagctaacatgtacagtcaaatagaaaaaaactatctacgcacaaatgtt |
| | tcagaacttcaaactatgtgcccttcagaagtaacaaatatggaaatcacatctgaacaaaacaagggga |
| | gtttgaacaatattgtcgagggaactgaaaaacaatctcacagtcaatctacttcactgtaa |
| 2 | MFRQFYLWTCLASGIILGSLFEICLGQYDDDWQYEDCKLARGGPP |
| | ATIVAIDEESRNGTILVDNMLIKGTAGGPDPTIELSLKDNVDYWVL |
| | MDPVKQMLFLNSTGRVLDRDPPMNIHSIVVQVQCINKKVGTIIYH |
| | EVRIVVRDRNDNSPTFKHESYYATVNELTPVGTTIFTGFSGDNGAT |
| | DIDDGPNGQIEYVIQYNPDDPTSNDTFEIPLMLTGNIVLRKRLNYE |
| | DKTRYFVIIQANDRAQNLNERRTTTTTLTVDVLDGDDLGPMFLPC |
| | VLVPNTRDCRPLTYQAAIPELRTPEELNPIIVTPPIQAIDQDRNIQPP |
| | SDRPGILYSILVGTPEDYPRFFHMHPRTAELSLLEPVNRDFHQKFD |
| | LVIKAEQDNGHPLPAFAGLHIEILDENNQSPYFTMPSYQGYILESAP |
| | VGATISDSLNLTSPLRIVALDKDIEDTKDPELHLFLNDYTSVFTVTQ |
| | TGITRYLTLLQPVDREEQQTYTFSITAFDGVQESEPVIVNIQVMDA |
| | NDNTPTFPEISYDVYVYTDMRPGDSVIQLTAVDADEGSNGEITYEI |
| | LVGAQGDFIINKTTGLITIAPGVEMIVGRTYALTVQAADNAPPAER |
| | RNSICTVYIEVLPPNNQSPPRFPQLMYSLEISEAMRVGAVLLNLQA |
| | TDREGDSITYAIENGDPQRVFNLSETTGILTLGKALDRESTDRYILII |
| | TASDGRPDGTSTATVNIVVTDVNDNAPVFDPYLPRNLSVVEEEAN |
| | AFVGQVKATDPDAGINGQVHYSLGNFNNLFRITSNGSIYTAVKLN |
| | REVRDYYELVVVATDGAVHPRHSTLTLAIKVLDIDDNSPVFTNST |
| | YTVLVEENLPAGTTILQIEAKDVDLGANVSYRIRSPEVKHFFALHP |
| | FTGELSLLRSLDYEAFPDQEASITFLVEAFDIYGTMPPGIATVTVIV |
| | KDMNDYPPVFSKRIYKGMVAPDAVKGTPITTVYAEDADPPGLPAS |
| | RVRYRVDDVQFPYPASIFEVEEDSGRVITRVNLNEEPTTIFKLVVV |
| | AFDDGEPVMSSSATVKILVLHPGEIPRFTQEEYRPPPVSELATKGT |
| | MVGVISAAAINQSIVYSIVSGNEEDTFGINNITGVIYVNGPLDYETR |
| | TSYVLRVQADSLEVVLANLRVPSKSNTAKVYIEIQDENNHPPVFQ |
| | KKFYIGGVSEDARMFTSVLRVKATDKDTGNYSVMAYRLIIPPIKE |
| | GKEGFVVETYTGLIKTAMLFHNMRRSYFKFQVIATDDYGKGLSG |
| | KADVLVSVVNQLDMQVIVSNVPPTLVEKKIEDLTEILDRYVQEQIP |
| | GAKVVVESIGARRHGDAFSLEDYTKCDLTVYAIDPQTNRAIDRNE |
| | LFKFLDGKLLDINKDFQPYYGEGGRILEIRTPEAVTSIKKRGESLGY |
| | TEGALLALAFIIILCCIPAILVVLVSYRQFKVRQAECTKTARIQAAL |
| | PAAKPAVPAPAPVAAPPPPPPPPPGAHLYEELGDSSMHNLFLLYHF |
| | QQSRGNNSVSEDRKHQQVVMPFSSNTIEAHKSAHVDGSLKSNKL |
| | KSARKFTFLSDEDDLSAHNPLYKENISQVSTNSDISQRTDFVDPFSP |
| | KIQAKSKSLRGPREKIQRLWSQSVSLPRRLMRKVPNRPEIIDLQQW |
| | QGTRQKAENENTGICTNKRGSSNPLLTTEEANLTEKEEIRQGETLM |
| | IEGTEQLKSLSSDSSFCFPRPHFSFSTLPTVSRTVELKSEPNVISSPAE |
| | CSLELSPSRPCVLHSSLSRRETPICMLPIETERNIFENFAHPPNISPSA |
| | CPLPPPPPISPPSPPPAPAPLAPPPDISPFSLFCPPPSPPSIPLPLPPPTFF |
| | PLSVSTSGPPTPPLLPPFPTPLPPPPPSIPCPPPPSASFLSTECVCITGV |
| | KCTTNLMPAEKIKSSMTQLSTTTVCKTDPQREPKGILRHVKNLAE |
| | LEKSVANMYSQIEKNYLRTNVSELQTMCPSEVTNMEITSEQNKGS |
| | LNNIVEGTEKQSHSQSTSL |
| 3 | atgtt tcgacagttt tatctctgga catgtttagc ttcagggatc atcctgggct ctctctttga |
| | aatctgcttg ggccagtatg atgatgactg gcaatatgag gattgcaaac tagctagggg |
| | aggaccacca gctaccatag ttgctattga tgaagaaagt cggaatggta caattctggt |
| | ggacaacatg ctgatcaaag ggactgctgg aggaccagac cccaccatag aactttcttt |
| | aaaggataat gtggattactgggtgttgat ggatcctgtt aagcaaatgc ttttcctgaa |
| | cagcaccgga agagttctgg atagagatcc accgatgaac atacactcca ttgtggtgca |
| | ggtccagtgc atcaacaaaaaagtgggcac tattatctac catgaagtgc gaatagtggt |
| | gagagacagg aatgacaactcacccacttt caagcatgaa agctactatg ccacagtgaa |
| | tgagctcact ccagttggtaccacaatatt cacaggattt tcaggagaca atggagctac |
| | agatatagat gatggaccaa atggacagat agagtatgtt attcagtata atccagatga |
| | tccgacatcc aatgacacctttgaaattcc cctaatgttg actggaaata tagtgttaag |
| | gaagaggctc aactatgaagataagactcg ctactttgtc ataatccaag ctaatgaccg |
| | tgcccaaaat ctgaatgagaggcgaaccac caccaccact ctcacagtgg atgttctgga |
| | tggagatgac ttgggtccaa tgtttcttcc ttgtgtcctt gtgccaaaca ctcgtgattg ccgtccactc |
| | acttatcaagctgccatacc tgagttgaga actccggaag aactgaaccc cattattgtt |
| | acgccaccaatccaagccat tgatcaggac cggaatattc aaccgccatc agataggcca |
| | ggaatcctctattccatcct tgttgggact cctgaggatt acccacgatt tttccatatg |
| | catcctaggacagcagaact tagtctcctg gagccagtaa acagagactt tcaccagaaa |
| | tttgatttggttattaaggc tgaacaagac aatggtcatc ctcttcctgc ctttgccggt |
| | ctacacattgaaatactgga tgaaaacaat caaagtccat attttacaat gcccagttat |
| | caaggctatatcctggaatc tgccccagtg ggagcaacca tttcggacag tctcaatttg |
| | acttcacctttaagaatagt agctctggac aaggacatag aagatgttcc acccagtgga |
| | gttcctacaaaagacccaga gcttcacctt tttctgaatg actacacctc agtcttcacc |
| | gtcacacagactggtattac tcgctacctc accttacttc aaccagtgga cagggaagaa |
| | cagcaaacttacaccttttc gataacagca tttgatggtg tacaagaaag tgagccagtc |
| | atcgtcaatattcaagtgat ggatgcaaat gataacacgc caaccttccc tgaaatatcc |
| | tatgatgtgtatgtttatac agacatgaga cctggggaca gtgtcataca gctcactgca |
| | gtcgacgcagacgaagggtc aaatggggag atcacatatg aaatccttgt tggggctcag |
| | ggagacttcatcatcaataa aacaacaggg cttatcacca tcgctccagg ggtggaaatg |
| | atagtcgggcggacttacgc actcacggtc caagcagcgg ataatgctcc tcctgcagag |
| | cgaaggaactccatctgcac tgtgtatatt gaagtgcttc caccaaataa tcaaagccct |
| | cctcgcttcccacagctgat gtatagcctt gaaattagtg aagccatgag ggttggtgct |
| | gttttattaaatctacaggc aactgatcga gagggagact caataacata tgccattgag |
| | aatggagatcctcagagagt ttttaatctt tcagaaacca cggggattct aaccttaggg |
| | aaagcactggacagggaaag cactgatcgc tacattctga tcatcacagc ttcagatggc |
| | aggccagatg ggacctcaac tgccacagta aacatagtgg tgacagatgt caatgacaat |
| | gctccagtgtttgatcctta tctgccaaga aatttatctg tggtggaaga agaagccaat |
| | gcctttgtgggtcaagtaaa agcaacagac cctgatgctg gaataaatgg tcaagtgcac |
| | tacagtttgggtaactttaa taatcttttt cgtatcacat ccaatgggag catttacaca gcagtgaagc |
| | ttaacagaga agtcagggac tactatgaac ttgttgttgt ggcaacagat |
| | ggagcagtacaccctcgtca ttcaactcta accttggcca tcaaggtttt ggacattgat |
| | gataacagtcctgtgttcac caattcaaca tacactgtcc ttgttgaaga gaatttgcca |
| | gctgggactaccatccttca aatagaggcc aaagatgtcg accttggagc aaatgtgtct |
| | taccggataagaagcccaga agtgaagcac ttttttgcac tacatccatt tacaggagaa |
| | ctatcgcttttaaggagttt agattatgag gcatttccag accaagaagc aagtatcact |
| | tttctggtagaggcctttga tatttatgga acaatgccac ctggtattgc tactgtcaca gtgattgtaa |
| | aggatatgaa tgattatcct cctgtcttta gtaaacgaat atacaaaggg |
| | atggtggctccggatgcagt caagggtaca cctatcacaa cagtttatgc tgaagatgca |
| | gaccctcctggattacctgc aagtcgtgtg aggtatagag tagatgatgt acagtttcct |
| | taccctgccagtatttttga agtggaagaa gattctggaa gagtaataac acgagtcaat |
| | cttaatgaag aacctacaac aatttttaag ttggtggtgg ttgcttttga tgatggggag |
| | cctgtgatgtccagcagtgc cacagtgaag attcttgtct tacatcctgg tgagatccca |
| | cgcttcacacaggaggaata tagacctcct ccagtaagtg aacttgccac caaagggacc |
| | atggttggtgtaatttctgc tgctgccatt aatcaaagta ttgtgtactc cattgtttca |
| | ggaaatgaagaagatacatt tggaattaat aacatcacag gtgttatcta tgtgaatgga |
| | cctctggattatgagaccag gacaagctat gtacttcgag tccaagctga ttccctggaa |
| | gtggtccttgccaatctccg agttccttca aaaagcaata cagctaaagt atacattgag |
| | attcaggatg aaaataatca tcccccagtg tttcagaaaa aattctacat cggaggtgta |
| | tctgaagatg caagaatgtt tacttctgta ctcagagtga aggctactga taaagatact |
| | ggcaattatagtgtcatggc ctacagactc ataataccac caattaaaga gggaaaagaa |
| | ggatttgtagtggaaacata tacagggctt atcaaaactg ctatgctctt ccataatatg aggagatcct |
| | acttcaagtt tcaagttatt gcaactgacg actatgggaa gggactgagc |
| | ggcaaagccgatgtactcgt ctccgtggtc aatcagctgg atatgcaagt cattgtttcc |
| | aatgtgcctcctactctagt ggaaaaaaag atagaagatc ttacagagat cttggatcgc |
| | tatgttcaggaacaaattcc tggtgccaag gtcgtagtgg agtccattgg agctcgccgg |
| | catggagatg ccttttccct agaagattac accaaatgtg acttgactgt ctatgcaatt |
| | gacccccaaaccaacagagc catcgataga aatgagcttt ttaaattttt ggatggcaaa |
| | ctacttgatatcaataaaga ctttcagccg tattatgggg aaggaggacg cattctggag |
| | atccggactccagaggcagt gaccagcatt aaaaagagag gagaaagtct aggatacaca |
| | gaaggggcct tgttggctct ggccttcatc atcatcctct gctgcattcc tgccatcttg |
| | gtggttttggtcagctacag acagtttaaa gtacgtcaag ctgagtgtac aaagactgca |
| | cgaattcaggccgcattacc cgcggctaaa ccagcagtgc cggctcctgc accagtggca |
| | gcgcccccgccgccgccgcc gcctccgcca ggtgcgcatc tctatgaaga acttggagac |
| | agctcaatgc ataagtatga aatgcctcaa tatgggagtc gccgtcgatt gttaccacca |
| | gctggacaggaggaatatgg tgaggtggtt ggtgaagctg aggaagaata tgaggaggaa |
| | gaggaagagccaaagaaaat taaaaaacca aaggttgaaa ttagagagcc tagtgaggag |
| | gaagaagtagttgtaactat cgaaaaacca ccagcagctg agcctacata cacaacatgg |
| | aagagagcca gaatattccc catgattttt aagaaagtta gaggattagc tgataaaaga |
| | ggaatcgttgaccttgaggg tgaagagtgg cagagacgcc ttgaggaaga agataaagat |
| | tatttgaaactcactctgga ccaagaggaa gcaacagaaa gcactgtaga atcagaggag |
| | gaatcctccagcgactatac tgaatacagt gaagaagagt ctgagttcag tgagtctgag |
| | actacagaag aggaatctga gtcagagaca ccctctgagg aggaggagag ttccacccct |
| | gaatcagaagaatcggaatc cacagagtca gaaggagaaa aagcaaggaa aaacattgtg |
| | cttgcaagaagaaggcccat ggttgaggag gtcaaggaag tcaagggtag gaaagaggag |
| | ccacaagaagaacaaaaaga acctaagatg gaagaagaag aacactcaga agaagaagaa |
| | agtggaccag cccctgtgga agaaagtaca gaccctgaag ctcaagatat ccctgaagag |
| | ggcagtgcagaatcagcttc ggtggaagga ggtgtggaaa gtgaggagga atcagaatca |
| | ggtagtagtagcagtagtag cgaaagtcag tctggaggtc catggggcta tcaggtacca |
| | gcgtatgacagaagcaagaa tgcaaaccaa aagaagtcgc caggagcaaa ctctgaaggt |
| | tacaacacag cactttga |
| 4 | MFRQFYLWTCLASGIILGSLFEICLGQYDDDWQYEDCKLARGGPP |
| | ATIVAIDEESRNGTILVDNMLIKGTAGGPDPTIELSLKDNVDYWVL |
| | MDPVKQMLFLNSTGRVLDRDPPMNIHSIVVQVQCINKKVGTIIYH |
| | EVRIVVRDRNDNSPTFKHESYYATVNELTPVGTTIFTGFSGDNG |
| | ATDIDDGPNGQIEYVIQYNPDDPTSNDTFEIPLMLTGNIVLRKRLN |
| | YEDKTRYFVIIQANDRAQNLNERRTTTTTLTVDVLDGDDLGPMFL |
| | PCVLVPNTRDCRPLTYQAAIPELRTPEELNPIIVTPPIQAIDQDRNIQ |
| | PPSDRPGILYSILVGTPEDYPRFFHMHPRTAELSLLEPVNRDFHQKF |
| | DLVIKAEQDNGHPLPAFAGLHIEILDENNQSPYFTMPSYQGYILES |
| | APVGATISDSLNLTSPLRIVALDKDIEDVPPSGVPTKDPELHLFLND |
| | YTSVFTVTQTGITRYLTLLQPVDREEQQTYTFSITAFDGVQESEPVI |
| | VNIQVMDANDNTPTFPEISYDVYVYTDMRPGDSVIQLTAVDADE |
| | GSNGEITYEILVGAQGDFIINKTTGLITIAPGVEMIVGRTYALTVQA |
| | ADNAPPAERRNSICTVYIEVLPPNNQSPPRFPQLMYSLEISEAMRV |
| | GAVLLNLQATDREGDSITYAIENGDPQRVFNLSETTGILTLGKALD |
| | RESTDRYILIITASDGRPDGTSTATVNIVVTDVNDNAPVFDPYLPR |
| | NLSVVEEEANAFVGQVKATDPDAGINGQVHYSLGNFNNLFRITSN |
| | GSIYTAVKLNREVRDYYELVVVATDGAVHPRHSTLTLAIKVLDID |
| | DNSPVFTNSTYTVLVEENLPAGTTILQIEAKDVDLGANVSYRIRSP |
| | EVKHFFALHPFTGELSLLRSLDYEAFPDQEASITFLVEAFDIYGTMP |
| | PGIATVTVIVKDMNDYPPVFSKRIYKGMVAPDAVKGTPITTVYAE |
| | DADPPGLPASRVRYRVDDVQFPYPASIFEVEEDSGRVITRVNLNEE |
| | PTTIFKLVVVAFDDGEPVMSSSATVKILVLHPGEIPRFTQEEYRPPP |
| | VSELATKGTMVGVISAAAINQSIVYSIVSGNEEDTFGINNITGVIYV |
| | NGPLDYETRTSYVLRVQADSLEVVLANLRVPSKSNTAKVYIEIQD |
| | ENNHPPVFQKKFYIGGVSEDARMFTSVLRVKATDKDTGNYSVMA |
| | YRLIIPPIKEGKEGFVVETYTGLIKTAMLFHNMRRSYFKFQVIATD |
| | DYGKGLSGKADVLVSVVNQLDMQVIVSNVPPTLVEKKIEDLTEIL |
| | DRYVQEQIPGAKVVVESIGARRHGDAFSLEDYTKCDLTVYAIDPQ |
| | TNRAIDRNELFKFLDGKLLDINKDFQPYYGEGGRILEIRTPEAVTSI |
| | KKRGESLGYTEGALLALAFIIILCCIPAILVVLVSYRQFKVRQAECT |
| | KTARIQAALPAAKPAVPAPAPVAAPPPPPPPPPGAHLYEELGDSSM |
| | HKYEMPQYGSRRRLLPPAGQEEYGEVVGEAEEEYEEEEEEPKKIK |
| | KPKVEIREPSEEEEVVVTIEKPPAAEPTYTTWKRARIFPMIFKKVRG |
| | LADKRGIVDLEGEEWQRRLEEEDKDYLKLTLDQEEATESTVESEE |
| | ESSSDYTEYSEEESEFSESETTEEESESETPSEEEESSTPESEESESTE |
| | SEGEKARKNIVLARRRPMVEEVKEVKGRKEEPQEEQKEPKMEEE |
| | EHSEEEESGPAPVEESTDPEAQDIPEEGSAESASVEGGVESEEESES |
| | GSSSSSSESQSGGPWGYQVPAYDRSKNANQKKSPGANSEGYNTA |
| | L |

The nucleic acid vectors (e.g., AAV vectors) used in the compositions and methods described herein include polynucleotide sequences that encode various isoforms of Pcdh15, or variants thereof, such as polynucleotide sequences that, when combined, encodes a protein having at least 85% sequence identity (e.g., 85%, 90%, 95%, 96%, 97%, 98%, 99%, or more, sequence identity) to the amino acid sequence of Pcdh15 corresponding to SEQ ID NO:2 or SEQ ID NO:4.

According to the methods described herein, a subject can be administered a composition containing a first nucleic acid vector and a second nucleic acid vector that contain an N-terminal and C-terminal portion, respectively, of a polynucleotide sequence encoding the amino acid sequence of SEQ ID NOS: 2 or 4, or a polynucleotide sequence encoding an amino acid sequence having at least 85% sequence identity (e.g., 85%, 90%, 95%, 96%, 97%, 98%, 99%, or more, sequence identity) to the amino acid sequence of SEQ ID NOS: 2 or 4, or a polynucleotide sequence encoding an amino acid sequence that contains one or more conservative amino acid substitutions relative to SEQ ID NOS: 2 or 4 (e.g., 1, 2, 3, 4, 5, 6, 7, 8, 9, 10, 11, 12, 13, 14, 15, 16, 17, 18, 19, 20 or more conservative amino acid substitutions), provided that the variant encoded retains the therapeutic function of Pcdh15. In some embodiments, the variant comprises an amino acid sequence that is at least 90% identical to SEQ ID NO:2 or 4.

In some embodiments, no more than 10% of the amino acids in the N-terminal portion of the human Pcdh15 and no more than 10% of the amino acids in the C-terminal portion of the human Pcdh15 may be replaced with conservative amino acid substitutions. In some embodiments, the Pcdh15 may be encoded by a polynucleotide having the sequence of SEQ ID NOS: 1 or 3. Variants of the polynucleotide sequence can also be used, for example, due to degeneracy of codon usage.

The organismal source of the nucleic acid sequence encoding Pcdh15 is not limiting. In some embodiments, Pcdh15 can be a homolog of human Pcdh15 from another mammalian species (e.g., mouse, rat, cow, horse, goat, sheep, donkey, cat, dog, rabbit, guinea pig, or other mammal). In some embodiments, the nucleic acid sequence is derived from a mammal. In some embodiments, the nucleic acid sequence is of human origin.

The nucleic acid molecules that can comprise the first or second vector can be produced using recombinant DNA technology (e.g., polymerase chain reaction (PCR) amplification, cloning) or chemical synthesis. Nucleic acids that encode Pcdh15 include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect (e.g., production of Pcdh15 protein in cells or other expression systems).

In some embodiments, the coding sequence of Pcdh15 is encoded by SEQ ID NOS: 1 or 3. The nucleic acid encoding Pcdh15 in accordance with the invention may contain a variety of different bases compared to the wild-type sequence and yet still encode a corresponding polypeptide that exhibits the biological activity of the native Pcdh15 polypeptide.

In some embodiments, a particular nucleotide sequence encoding Pcdh15 polypeptide may be identical over its entire length to the coding sequence in SEQ ID NOS: 1 or 3. In some embodiments, a particular nucleotide sequence encoding Pcdh15 polypeptide may be an alternate form of SEQ ID NOS: 1 or 3 due to degeneracy in the genetic code or variation in codon usage encoding the polypeptide of SEQ ID NOS: 2 or 4.

In some embodiments, the nucleic acid sequence of Pcdh15 can contain a nucleotide sequence that is highly identical, at least 60% identical, with a nucleotide sequence encoding Pcdh15 polypeptide. In some embodiments, the nucleic acid sequence of Pcdh15 comprises a nucleotide sequence that is at least 60%, 65%, 70%, 75%, 80%, 85%, 90%, 91%, 92%, 93%, 94%, 95%, 96%, 97%, 98% 99% or 100% identical with the encoding nucleotide sequence set forth in SEQ ID NOS: 1 or 3.

When a polynucleotide of the invention is used for the production of Pcdh15 polypeptide, the polynucleotide may include the coding sequence for the full-length polypeptide or a fragment thereof, by itself; the coding sequence for the full-length polypeptide or fragment in reading frame with other coding sequences, such as those encoding a leader or secretory sequence, a pre-, or pro or prepro-protein sequence, or other fusion peptide portions. The polynucleotide may also contain non-coding 5′ and 3′ sequences, such as transcribed, non-translated sequences, splicing and polyadenylation signals, ribosome binding sites and sequences that stabilize mRNA.

In some embodiments, the dual nucleotide sequences used in the dual vector system encoding the Pcdh15 peptide or a biologically active fragment or derivative thereof includes nucleic acid molecules comprising a polynucleotide having a nucleotide sequence at least 90%, 91%, 92%, 93%, 94%, 95%, 96%, 97%, 98% 99% or 100% identical to (a) a nucleotide sequence encoding Pcdh15 having the amino acid sequence in SEQ ID NOS: 2 or 4; or (b) a nucleotide sequence complementary to the nucleotide sequences in (a).

Conventional means utilizing known computer programs such as the BestFit program (Wisconsin Sequence Analysis Package, Version 10 for Unix, Genetics Computer Group, University Research Park, 575 Science Drive, Madison, Wis. 53711) may be utilized to determine if a particular nucleic acid molecule is at least 90%, 91%, 92%, 93%, 94%, 95%, 96%, 97%, 98% or 99% identical to SEQ ID NOS: 1 or 3, for example.

In some embodiments, the nucleotide sequences used in the dual vector system encoding Pcdh15 or a variant thereof encodes an amino acid sequence of Pcdh15 of SEQ ID NOS: 2 or 4, in which 1, 2, 3, 4, 5, 6, 7, 8, 9, 10 or more amino acid residues are substituted, deleted or added, in any combination.

In some embodiments, the nucleotide sequences (when considered after splicing and/or recombination) are at least 90% identical over their entire length to a polynucleotide encoding the corresponding portion of Pcdh15, for which the amino acid sequences are set out in SEQ ID NOS: 2 or 4, and polynucleotides which are complementary to such polynucleotides. In some embodiments, the polynucleotides are at least 91%, 92%, 93%, 94%, 95%, 96%, 97%, 98%, or at least 99% identical.

In some embodiments, the nucleic acid molecule (when considered after splicing and/or recombination) encodes a variant of Pcdh15 protein which is a biologically active fragment. In some embodiments, the biologically active fragment can be at least about 1600, 1700, 1800, or 1900 amino acids in length.

Stable expression of Pcdh15 or variant thereof in a mammalian cell can be achieved by integration of the polynucleotides containing the Pcdh15 or variant thereof into the nuclear genome of the mammalian cell. A variety of vectors for the delivery and integration of polynucleotides encoding exogenous proteins into the nuclear DNA of a mammalian cell have been developed. Examples of expression vectors are disclosed in, e.g., WO 1994/011026 and are incorporated herein by reference. In some embodiments, expression vectors for use in the compositions and methods described herein contain a polynucleotide sequence that encodes a portion of Pcdh15 or variant thereof, as well as, e.g., additional sequence elements used for the expression of these agents and/or the integration of these polynucleotide sequences into the genome of a mammalian cell. Certain vectors that can be used for the expression of Pcdh15 include plasmids that contain regulatory sequences, such as promoter and enhancer regions, which direct gene transcription. Other useful vectors for expression of Pcdh15 contain polynucleotide sequences that enhance the rate of translation of these genes or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

In some embodiments, the compositions and methods described herein increase the expression of Pcdh15 or variants thereof by administering a first nucleic acid vector that contains a polynucleotide encoding an N-terminal portion of an Pcdh15 and a second nucleic acid vector that contains a polynucleotide encoding a C-terminal portion of an Pcdh15. In order to utilize nucleic acid vectors for therapeutic application in the treatment of conditions described herein, they can be directed to the interior of the cell, and, in particular, to specific cell types.

Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

In some embodiments, Pcdh15 or variants thereof can also be introduced into a mammalian cell by targeting vectors containing portions of a gene encoding Pcdh15 or a variant thereof to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding a Pcdh15 by mammalian RNA polymerase is important for gene expression. As such, one may include sequence elements within the polynucleotide that exhibit a high affinity for transcription factors that recruit RNA polymerase and promote the assembly of the transcription complex at the transcription initiation site.

Such sequence elements include, e.g., a mammalian promoter, the sequence of which can be recognized and bound by specific transcription initiation factors and ultimately RNA polymerase.

Polynucleotides suitable for use in the compositions and methods described herein also include those that encode an Pcdh15 protein downstream of a mammalian promoter (e.g., a polynucleotide that encodes an N-terminal portion of an Pcdh15 downstream of a mammalian promoter). Promoters that are useful for the expression of an Pcdh15 protein in mammalian cells include ubiquitous promoters and cochlear hair cell-specific promoters. Ubiquitous promoters include the CAG promoter, or the cytomegalovirus (CMV) promoter. Cell type and tissue specific promoters can also be utilized.

Alternatively, promoters derived from viral genomes can also be used for the stable expression of these agents in mammalian cells. Examples of functional viral promoters that can be used to promote mammalian expression of these agents include adenovirus late promoter, vaccinia virus 7.5K promoter, SV40 promoter, tk promoter of HSV, mouse mammary tumor virus (MMTV) promoter, LTR promoter of HIV, promoter of Moloney virus, Epstein barr virus (EBV) promoter, and the Rous sarcoma virus (RSV) promoter.

Pcdh15 or a variant thereof (e.g., Pcdh15 having the amino acid sequence of SEQ ID NOS: 2 or 4) can be expressed in mammalian cells using a dual hybrid vector system. This approach uses two nucleic acid vectors (e.g., two adeno-associated virus vectors) to express a single, large protein. Each of the two nucleic acid vectors (e.g., two adeno-associated virus vectors) contains a portion of a polynucleotide that encodes the protein (e.g., one vector contains a polynucleotide encoding an N-terminal portion of the protein and the other vector contains a polynucleotide encoding a C-terminal portion of the protein, and the polynucleotide encoding the N-terminal portion of the protein and the polynucleotide encoding the C-terminal portion of the protein do not overlap). In some embodiments, the dual hybrid vectors can also feature an overlapping region at which homologous recombination can occur (e.g., a recombinogenic region that is contained within each vector) and splice donor and splice acceptor sequences (e.g., the first vector contains a splice donor sequence and the second vector contains a splice acceptor sequence).

In some embodiments, the recombinogenic region is 3′ of the splice donor sequence in the first nucleic acid vector and 5′ of the splice acceptor sequence in the second nucleic acid vector. In some embodiments, the first and second polynucleotide sequences can then join to form a single sequence based on one of two mechanisms: 1) recombination at the overlapping region, or 2) concatemerization of the ITRs. The remaining recombinogenic region(s) and/or the concatemerized ITRs can be removed by splicing, leading to the formation of a contiguous polynucleotide sequence that encodes the full-length protein of interest.

In some embodiments, the first and second vectors of the dual vector system are viral vectors. A “viral vector” is a virus that can be used to deliver genetic material into target cells. This can be done either in vivo or in vitro. In general, viral vectors are either inherently safe or are modified to present a low handling risk and have low toxicity with respect to the targeted cells. A “retrovirus” is a virus of the family Retroviridae that inserts a copy of its RNA genome into the DNA of a host cell, then uses a reverse transcriptase enzyme to produce DNA from its RNA genome. Retroviruses are known in the art to be useful in gene delivery systems. A “lentivirus” is a type of retrovirus; they are known as slow retroviruses. They are associated with severe immunodeficiency and death in humans but can be useful as viral vectors in gene therapy. An “adenovirus” is a virus of the family Adenoviridae that lacks an outer lipid bilayer and includes a double stranded DNA genome. Adenoviruses are well established in the art as viral vectors for gene therapy, and delivering genes coding proteins of interest to particular locations, as to selected cell types, is possible. An “adeno-associated virus” is of the genus Dependoparvovirus, which is of the family Parvoviridae. These are nonenveloped viruses having a single-stranded DNA genome. Adeno-associated viruses are well known in the art as attractive candidates for use as viral vectors for gene therapy. Unlike adenoviruses, they have the advantage that they do not cause disease.

In some embodiments, nucleic acids of the compositions and methods described herein are incorporated into recombinant AAV (rAAV) vectors and/or virions in order to facilitate their introduction into a cell. rAAV vectors useful in the compositions and methods described herein are recombinant nucleic acid constructs that include (1) a heterologous sequence to be expressed (e.g., a polynucleotide encoding an N-terminal or C-terminal portion of a Pcdh15 protein or variant thereof) and (2) viral sequences that facilitate stability and expression of the heterologous genes. The viral sequences may include those sequences of AAV that are required in cis for replication and packaging (e.g., functional ITRs) of the DNA into a virion. Such rAAV vectors may also contain marker or reporter genes. In some embodiments, useful rAAV vectors have one or more of the AAV wild-type genes deleted in whole or in part, but retain functional flanking ITR sequences. The AAV ITRs may be of any serotype suitable for a particular application. In some embodiments, the ITRs can be AAV2 ITRs. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery. rAAV virions useful in conjunction with the compositions and methods described herein include those derived from a variety of AAV serotypes including, without limitation, AAV1, AAV2, AAV2quad (Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S. In some embodiments, for targeting inner ear or retinal cells, the first and/or second vector are adeno-associated viral (AAV) vectors selected from AAV2/2, AAV2/5, AAV2/9, AAV2/Anc80, AAV-7m8 and R100.

The first and second nucleic acid vectors (e.g., AAV vectors) in the compositions and methods described herein may have the same serotype or different serotypes. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

The use of AAV vectors for delivering a functional Pcdh15 requires the use of a dual vector system, in in which the first member of the dual vector system encodes an N-terminal portion of an Pcdh15 and the second member encodes a C-terminal portion of an Pcdh15 such that, upon administration of the dual vector system to a cell, the polynucleotide sequences contained within the two vectors can join to form a single sequence that results in the production of a full-length Pcdh15.

In some embodiments, the first and/or second vector can comprise various sequence motifs that aid in integration, recombination, splicing and/or expression. In some embodiments, the first vector of the dual vector system can include, in 5′ to 3′ order, a first inverted terminal repeat (“ITR”); a promoter (e.g., a CMV promoter); a Kozak sequence; an N-terminal portion of an Pcdh15 coding sequence; a splice donor sequence; an AP gene fragment (e.g., an AP head sequence); and a second ITR; and the second member of the dual vector system can include, in 5′ to 3′ order, a first ITR; an AP gene fragment (e.g., an AP head sequence); a splice acceptor sequence; a C-terminal portion of an Pcdh15 coding sequence; a polyA sequence; and a second ITR. In some embodiments, the N-terminal portion of the Pcdh15 coding sequence and the C-terminal portion of the Pcdh15 coding sequence do not overlap and are joined in a cell (e.g., by recombination at the overlapping region (the AP gene fragment), or by concatemerization of the ITRs) to produce the full-length Pcdh15 amino sequence as set forth in SEQ ID NOS: 2 or 4 or variants thereof. Sequence motifs that can be useful in the dual vector systems herein are shown below in Table 2.

TABLE 2
Vector sequence motifs.

| Function | Sequence |
|---|---|
| 5′ ITR (first and | ctgcgcgctcgctcgctcactgaggccgcccgggcaaagcccgggcgtcgggcgacctttg |
| second vector) | gtcgcccggcctcagtgagcgagcgagcgcgcagagagggagtggccaactccatcacta |
| | ggggttcct (SEQ ID NO: 5) |
| 3′ ITR (first and | aggaacccctagtgatggagttggccactccctctctgcgcgctcgctcgctcactgaggccg |
| second vector) | ggcgaccaaaggtcgcccgacgcccgggctttgcccgggcggcctcagtgagcgagcga |
| | gcgcgcagctgcctgcagg (SEQ ID NO: 6) |
| CMV promoter | cgttacataacttacggtaaatggcccgcctggctgaccgcccaacgacccccgcccattga |
| (first vector) | cgtcaataatgacgtatgttcccatagtaacgccaatagggactttccattgacgtcaatgggtg |
| | gagtatttacggtaaactgcccacttggcagtacatcaagtgtatcatatgccaagtccgcccc |
| | ctattgacgtcaatgacggtaaatggcccgcctggcattatgcccagtacatgaccttacggga |
| | ctttcctacttggcagtacatctacgtattagtcatcgctattaccatggtgatgcggttttggcag |
| | tacaccaatgggcgtggatagcggtttgactcacggggatttccaagtctccaccccattgac |
| | gtcaatgggagtttgttttggcaccaaaatcaacgggactttccaaaatgtcgtaataaccccgc |
| | cccgttgacgcaaatgggcggtaggcgtgtacggtgggaggtctatataagcagagctcgttt |
| | agtgaaccgt (SEQ ID NO: 7) |
| 5′ partial AP | gtcgacgcagggcagcctctgtcatctccatcagggaggggtccagtgtggagtctcggtgg |
| site (first | atctcgtatttcatgtctccaggctcaaagagacccatgagatgggtcacagacgggtccagg |
| vector) | gaagcatgcatgagctcagtgcggttccacacataccgggcaccctggcgcttcgccagcca |
| | ttcctgcaccagattcttcccgtccagcctggtcccaccttggctgtagtcatctgggtactcag |
| | ggtctggggttcccatgcgaaacatgtactttcggcctcca |
| | (SEQ ID NO: 8) |
| Splice donor | gtaagtatcaaggttacaagacaggtttaaggagaccaatagaaactgggcttgtcgagacag |
| sequence (first | agaagactcttgcgtttctgaggtaccgcatgcaccacggacc (SEQ ID NO: 9) |
| vector) | |
| 3′ partial AP | cgcagggcagcctctgtcatctccatcagggaggggtccagtgtggagtctcggtggatctc |
| site (second | gtatttcatgtctccaggctcaaagagacccatgagatgggtcacagacgggtccagggaag |
| vector) | catgcatgagctcagtgcggttccacacataccgggcaccctggcgcttcgccagccattcct |
| | gcaccagattcttcccgtccagcctggtcccaccttggctgtagtcatctgggtactcagggtct |
| | ggggttcccatgcgaaacatgtactttcggcctccagga |
| | (SEQ ID NO: 10) (underlined portion is the splice acceptor |
| | sequence) |
| Splice acceptor | cctaggatcacgcatgctcgcgataggcacctattggtcttactgacatccactttgcctttctct |
| site (second | ccacag (SEQ ID NO: 11) |
| vector) | |
| bGH poly A | gcctcgactgtgccttctagttgccagccatctgttgtttgcccctcccccgtgccttccttgacc |
| site (second | ctggaaggtgccactcccactgtcctttcctaataaaatgaggaaattgcatcgcattgtctgag |
| vector) | taggtgtcattctattctggggggtggggtggggcaggacagcaagggggaggattgggaa |
| | gacaatagcaggcatgctgggga (SEQ ID NO: 12) |

The N-terminal portion of the Pcdh15 coding sequence or variant thereof that can be used in the first vector is not limiting. In some embodiments, the N-terminal portion of the Pcdh15 coding sequence encodes amino acids 1-732 of SEQ ID NOS: 2 or 4.

In some embodiments, the first vector of the dual vector system includes a CMV promoter having a polynucleotide sequence comprising at least 85%, 86%, 87%, 88%, 89%, 90%, 91%, 92%, 93%, 94%, 95%, 96%, 97%, 98%, 99% or greater identity to SEQ ID NO: 7 operably linked to nucleotides that encode the N-terminal amino acids of the Pcdh15 or variant thereof (e.g., amino acids 1-732 of SEQ ID NOS: 2 or 4).

In some embodiments, the nucleotide sequence that encodes the N-terminal amino acids of the Pcdh15 is any nucleotide sequence that, by redundancy of the genetic code, encodes, e.g., amino acids 1-732 of SEQ ID NOS: 2 or 4. The nucleotide sequences that encode the Pcdh15 can be partially or fully codon-optimized for expression. In some embodiments, the first member of the dual vector system includes the Kozak sequence.

In some embodiments, the first vector comprises a splice donor signal sequence positioned 3′ of the first coding polynucleotide and the second vector comprises a splice acceptor signal sequence positioned 5′ of the second coding polynucleotide. In some embodiments, the splice donor signal sequence comprises SEQ ID NO:9 and the splice acceptor signal sequence comprises SEQ ID NO:11.

In some embodiments, the first vector comprises a sequence that promotes recombination that is positioned 3′ of the splice donor signal sequence, and the second vector comprises a sequence that promotes recombination that is positioned 5′ of splice acceptor signal sequence. In some embodiments, the sequence that promotes recombination in the first vector is a partial AP site comprising SEQ ID NO:8 and the sequence that promotes recombination in the second vector is a partial AP site comprising SEQ ID NO:10.

In some embodiments, the first vector comprises a first inverted terminal repeat (ITR) sequence at a position that is located 5′ of the first coding polynucleotide, and a second ITR sequence that is that is located 3′ of the first coding polynucleotide, and the second vector comprises a first inverted terminal repeat (ITR) sequence at a position that is located 5′ of the second coding polynucleotide, and a second ITR sequence that is that is located 3′ of the second coding polynucleotide.

In some embodiments, the promoter is located at a position that is between the first ITR sequence and the first coding polynucleotide and the second ITR is located at a position that is 3′ to the splice donor site on the first vector, and the first ITR is located at a position that is 5′ to the splice acceptor site and the second ITR is located at a position that is 3′ of a poly(A) sequence on the second vector.

In some embodiments, the first ITRs in the first vector and second vector have at least 80% sequence identity (at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) to SEQ ID NO:5, and the second ITRs in the first vector and second vector have at least 80% sequence identity (at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) to SEQ ID NO:6.

The second member of the dual vector system includes nucleotides that encode C-terminal amino acids of the Pcdh15 or variant thereof. The C-terminal amino acids are not particularly limiting. In some embodiments, the second vector encodes amino acids 733-1962 of SEQ ID NO:2 or 733-1790 of SEQ ID NO:4 immediately followed by a stop codon. In some embodiments, the nucleotide sequence that encodes the C-terminal amino acids of the Pcdh15 or variant thereof is any nucleotide sequence that, by redundancy of the genetic code, encodes amino acids 733-1962 of SEQ ID NO:2 or 733-1790 of SEQ ID NO: 4. The nucleotide sequences that encode Pcdh15 or a variant thereof can be partially or fully codon-optimized for expression. In some embodiments, the second member of the dual vector system includes the splice acceptor sequence corresponding to SEQ ID NO:11.

In some embodiments, the second member of the dual vector system comprises a sequence that promotes recombination and is a partial AP site comprising SEQ ID NO:10.

In some embodiments, the second vector comprises a poly(A) sequence. The poly(A) sequence is not particularly limiting. In some embodiments, the poly(A) sequence is a bovine growth hormone (bGH) poly(A) signal sequence. In some embodiments, the poly(A) sequence is at least 80% identical (at least 80%, 85%, 86%, 87%, 88%, 89%, 90%, 91%, 92%, 93%, 94%, 95%, 96%, 97%, 98%, 99%, or 100% sequence identity) to SEQ ID NO: 12.

The following table below shows various vector sequences of the disclosure.

TABLE 3
Vector sequences

| SEQ ID | |
|---|---|
| NO: | Vector Sequence |
| 13 | HBL Sequence-Human PCDH15-CD1 full sequence (Retina isoform) |
| | 5′ITR, 3′ITR, CMV Promotor, SD site +5′ partial AP site, PCDH15 DNA |
| | seq |
| | ctgcgcgctcgctcgctcactgaggccgcccgggcaaagcccgggcgtcgggcgacctttggtcgcc |
| | cggcctcagtgagcgagcgagcgcgcagagagggagtggccaactccatcactaggggttccttgtag |
| | ttaatgattaacccgccatgctacttatctacgtagccatgctctaggaagatcggaattcgcccttaagcta |
| | gcggcgcgccgtttaaaccgttacataacttacggtaaatggcccgcctggctgaccgcccaacgaccc |
| | ccgcccattgacgtcaataatgacgtatgttcccatagtaacgccaatagggactttccattgacgtcaatg |
| | ggtggagtatttacggtaaactgcccacttggcagtacatcaagtgtatcatatgccaagtccgccccctat |
| | tgacgtcaatgacggtaaatggcccgcctggcattatgcccagtacatgaccttacgggactttcctacttg |
| | gcagtacatctacgtattagtcatcgctattaccatggtgatgcggttttggcagtacaccaatgggcgtgg |
| | atagcggtttgactcacggggatttccaagtctccaccccattgacgtcaatgggagtttgttttggcacca |
| | aaatcaacgggactttccaaaatgtcgtaataaccccgccccgttgacgcaaatgggcggtaggcgtgta |
| | cggtgggaggtctatataagcagagctcgtttagtgaaccgtcagatcactagaagctttattgcggtagg |
| | ccgccaccatggatgtttcgacagttttatctctggacatgtttagcttcagggatcatcctgggctctctcttt |
| | gaaatctgcttgggccagtatgatgatgactggcaatatgaggattgcaaactagctaggggaggaccac |
| | cagctaccatagttgctattgatgaagaaagtcggaatggtacaattctggtggacaacatgctgatcaaa |
| | gggactgctggaggaccagaccccaccatagaactttctttaaaggataatgtggattactgggtgttgat |
| | ggatcctgttaagcaaatgcttttcctgaacagcaccggaagagttctggatagagatccaccgatgaaca |
| | tacactccattgtggtgcaggtccagtgcatcaacaaaaaagtgggcactattatctaccatgaagtgcga |
| | atagtggtgagagacaggaatgacaactcacccactttcaagcatgaaagctactatgccacagtgaatg |
| | agctcactccagttggtaccacaatattcacaggattttcaggagacaatggagctacagatatagatgat |
| | ggaccaaatggacagatagagtatgttattcagtataatccagatgatccgacatccaatgacacctttgaa |
| | attcccctaatgttgactggaaatatagtgttaaggaagaggctcaactatgaagataagactcgctactttg |
| | tcataatccaagctaatgaccgtgcccaaaatctgaatgagaggcgaaccaccaccaccactctcacagt |
| | ggatgttctggatggagatgacttgggtccaatgtttcttccttgtgtccttgtgccaaacactcgtgattgcc |
| | gtccactcacttatcaagctgccatacctgagttgagaactccggaagaactgaaccccattattgttacgc |
| | caccaatccaagccattgatcaggaccggaatattcaaccgccatcagataggccaggaatcctctattc |
| | catccttgttgggactcctgaggattacccacgatttttccatatgcatcctaggacagcagaacttagtctc |
| | ctggagccagtaaacagagactttcaccagaaatttgatttggttattaaggctgaacaagacaatggtcat |
| | cctcttcctgcctttgccggtctacacattgaaatactggatgaaaacaatcaaagtccatattttacaatgcc |
| | cagttatcaaggctatatcctggaatctgccccagtgggagcaaccatttcggacagtctcaatttgacttc |
| | acctttaagaatagtagctctggacaaggacatagaagatacaaaagacccagagcttcacctttttctga |
| | atgactacacctcagtcttcaccgtcacacagactggtattactcgctacctcaccttacttcaaccagtgga |
| | cagggaagaacagcaaacttacaccttttcgataacagcatttgatggtgtacaagaaagtgagccagtc |
| | atcgtcaatattcaagtgatggatgcaaatgataacacgccaaccttccctgaaatatcctatgatgtgtatg |
| | tttatacagacatgagacctggggacagtgtcatacagctcactgcagtcgacgcagacgaagggtcaa |
| | atggggagatcacatatgaaatccttgttggggctcagggagacttcatcatcaataaaacaacagggctt |
| | atcaccatcgctccaggggggaaatgatagtcgggcggacttacgcactcacggtccaagcagcggat |
| | aatgctcctcctgcagagcgaaggaactccatctgcactgtgtatattgaagtgcttccaccaaataatcaa |
| | agccctcctcgcttcccacagctgatgtatagccttgaaattagtgaagccatgagggttggtgctgttttat |
| | taaatctacaggcaactgatcgagagggagactcaataacatatgccattgagaatggagatcctcagag |
| | agtttttaatctttcagaaaccacggggattctaaccttagggaaagcactggacagggaaagcactgatc |
| | gctacattctgatcatcacagcttcagatggcaggccagatgggacctcaactgccacagtaaacatagt |
| | ggtgacagatgtcaatgacaatgctccagtgtttgatccttatctgccaagaaatttatctgtggtaagtatca |
| | aggttacaagacaggtttaaggagaccaatagaaactgggcttgtcgagacagagaagactcttgcgttt |
| | ctgaggtaccgcatgcaccacggaccgtcgacgcagggcagcctctgtcatctccatcagggaggggt |
| | ccagtgtggagtctcggtggatctcgtatttcatgtctccaggctcaaagagacccatgagatgggtcaca |
| | gacgggtccagggaagcatgcatgagctcagtgcggttccacacataccgggcaccctggcgcttcgc |
| | cagccattcctgcaccagattcttcccgtccagcctggtcccaccttggctgtagtcatctgggtactcagg |
| | gtctggggttcccatgcgaaacatgtactttcggcctccaggatcctagagcatggctaagatctcctatgc |
| | taggctcgagttaagggcgaattcccgataaggatcttcctagagcatggctacgtagataagtagcatg |
| | gcgggttaatcattaactacaaggaacccctagtgatggagttggccactccctctctgcgcgctcgctcg |
| | ctcactgaggccgggcgaccaaaggtcgcccgacgcccgggctttgcccgggcggcctcagtgagcg |
| | agcgagcgcg |
| 14 | HBR Sequence-Human PCDH15-CD1 full sequence (Retina isoform) |
| | 5′ITR, 3′ITR, SA site +3′ partial AP site, PCDH15 DNA seq, bGH pA |
| | ctgcgcgctcgctcgctcactgaggccgcccgggcaaagcccgggcgtcgggcgacctttggtcgcc |
| | cggcctcagtgagcgagcgagcgcgcagagagggagtggccaactccatcactaggggttccttgtag |
| | ttaatgattaacccgccatgctacttatctacgtagccatgctctaggaagatcggaattcgcccttaagcta |
| | gcttaattaacgcagggcagcctctgtcatctccatcagggaggggtccagtgtggagtctcggtggatct |
| | cgtatttcatgtctccaggctcaaagagacccatgagatgggtcacagacgggtccagggaagcatgca |
| | tgagctcagtgcggttccacacataccgggcaccctggcgcttcgccagccattcctgcaccagattcttc |
| | ccgtccagcctggtcccaccttggctgtagtcatctgggtactcagggtctggggttcccatgcgaaacat |
| | gtactttcggcctccaggacctaggatcacgcatgctcgcgataggcacctattggtcttactgacatcca |
| | ctttgcctttctctccacaggtggaagaagaagccaatgcctttgtgggtcaagtaaaagcaacagaccct |
| | gatgctggaataaatggtcaagtgcactacagtttgggtaactttaataatctttttcgtatcacatccaatgg |
| | gagcatttacacagcagtgaagcttaacagagaagtcagggactactatgaacttgttgttgtggcaacag |
| | atggagcagtacaccctcgtcattcaactctaaccttggccatcaaggttttggacattgatgataacagtc |
| | ctgtgttcaccaattcaacatacactgtccttgttgaagagaatttgccagctgggactaccatccttcaaat |
| | agaggccaaagatgtcgaccttggagcaaatgtgtcttaccggataagaagcccagaagtgaagcacttt |
| | tttgcactacatccatttacaggagaactatcgcttttaaggagtttagattatgaggcatttccagaccaaga |
| | agcaagtatcacttttctggtagaggcctttgatatttatggaacaatgccacctggtattgctactgtcacag |
| | tgattgtaaaggatatgaatgattatcctcctgtctttagtaaacgaatatacaaagggatggtggctccgga |
| | tgcagtcaagggtacacctatcacaacagtttatgctgaagatgcagaccctcctggattacctgcaagtc |
| | gtgtgaggtatagagtagatgatgtacagtttccttaccctgccagtatttttgaagtggaagaagattctgg |
| | aagagtaataacacgagtcaatcttaatgaagaacctacaacaatttttaagttggtggtggttgcttttgatg |
| | atggggagcctgtgatgtccagcagtgccacagtgaagattcttgtcttacatcctggtgagatcccacgc |
| | ttcacacaggaggaatatagacctcctccagtaagtgaacttgccaccaaagggaccatggttggtgtaat |
| | ttctgctgctgccattaatcaaagtattgtgtactccattgtttcaggaaatgaagaagatacatttggaattaa |
| | taacatcacaggtgttatctatgtgaatggacctctggattatgagaccaggacaagctatgtacttcgagt |
| | ccaagctgattccctggaagtggtccttgccaatctccgagttccttcaaaaagcaatacagctaaagtata |
| | cattgagattcaggatgaaaataatcatcccccagtgtttcagaaaaaattctacatcggaggtgtatctga |
| | agatgcaagaatgtttacttctgtactcagagtgaaggctactgataaagatactggcaattatagtgtcatg |
| | gcctacagactcataataccaccaattaaagagggaaaagaaggatttgtagtggaaacatatacagggc |
| | ttatcaaaactgctatgctcttccataatatgaggagatcctacttcaagtttcaagttattgcaactgacgact |
| | atgggaagggactgagcggcaaagccgatgtactcgtctccgtggtcaatcagctggatatgcaagtcat |
| | tgtttccaatgtgcctcctactctagtggaaaaaaagatagaagatcttacagagatcttggatcgctatgtt |
| | caggaacaaattcctggtgccaaggtcgtagtggagtccattggagctcgccggcatggagatgcctttt |
| | ccctagaagattacaccaaatgtgacttgactgtctatgcaattgacccccaaaccaacagagccatcgat |
| | agaaatgagctttttaaatttttggatggcaaactacttgatatcaataaagactttcagccgtattatgggga |
| | aggaggacgcattctggagatccggactccagaggcagtgaccagcattaaaaagagaggagaaagt |
| | ctaggatacacagaaggggccttgttggctctggccttcatcatcatcctctgctgcattcctgccatcttgg |
| | tggttttggtcagctacagacagtttaaagtacgtcaagctgagtgtacaaagactgcacgaattcaggcc |
| | gcattacccgcggctaaaccagcagtgccggctcctgcaccagtggcagcgcccccgccgccgccgc |
| | cgcctccgccaggtgcgcatctctatgaagaacttggagacagctcaatgcataatcttttccttctctacca |
| | ttttcaacaaagcaggggaaataactcagtctcagaagacaggaaacatcaacaagttgtgatgccctttt |
| | cttccaatactattgaggctcacaagtcagctcatgtagacggatcacttaagagcaacaaactgaagtct |
| | gcaagaaaattcacatttctatctgatgaggatgacttaagtgcccataatcccctttataaggaaaacataa |
| | gtcaagtatcaacaaattcagacatttcacagagaacagattttgtagacccattttcacccaaaatacaag |
| | ccaagagtaagtctctgaggggcccaagagaaaagattcagaggctgtggagtcagtcagtcagcttac |
| | ccaggaggctgatgaggaaagttccaaatagaccagagatcatagatctgcagcagtggcaaggcacc |
| | aggcagaaagctgaaaatgaaaacactggaatctgtacaaacaaaagaggtagcagcaatccattgctt |
| | acaactgaagaggcaaatttgacagagaaagaggaaataaggcaaggtgaaacactgatgatagaagg |
| | aacagaacagttgaaatctctctcttcagactcttcattttgctttcccaggcctcacttctcattctccactttg |
| | ccaactgtttcaagaactgtggaactcaaatcagaacctaatgtcatcagttctcctgctgagtgttccttgg |
| | aactttctccttcaaggccttgtgttttacattcttcactctctaggagagagacacctatttgtatgttacctatt |
| | gaaaccgaaagaaatatttttgaaaattttgcccatccaccaaacatctctccttctgcctgtccccttccccc |
| | tcctcctcctatttctcctccttctcctcctcctgctcctgctcctcttgctcctcctcctgacatttctcctttttct |
| | cttttttgtcctcctccctctcctccttctatccctcttcctcttcctcctcctacattttttccactttccgtttcaa |
| | cgtctggtcccccaacaccacctcttctacctccatttccaactcctcttcctccaccacctccttctattccttg |
| | ccctccacctccttcagcttcatttctgtccacagagtgtgtctgtataacaggtgttaaatgcacgaccaac |
| | ttgatgcctgccgagaaaattaagtcctctatgacacagctatcaacaacgacagtgtgtaaaacagaccc |
| | tcagagagaaccaaaaggcatcctcagacacgttaaaaacttagcagaacttgaaaaatcagtagctaac |
| | atgtacagtcaaatagaaaaaaactatctacgcacaaatgtttcagaacttcaaactatgtgcccttcagaa |
| | gtaacaaatatggaaatcacatctgaacaaaacaaggggagtttgaacaatattgtcgagggaactgaaa |
| | aacaatctcacagtcaatctacttcactgtaaactagtatttaaatcaattggcctcgactgtgccttctagttg |
| | ccagccatctgttgtttgcccctcccccgtgccttccttgaccctggaaggtgccactcccactgtcctttcc |
| | taataaaatgaggaaattgcatcgcattgtctgagtaggtgtcattctattctggggggtggggggggca |
| | ggacagcaagggggaggattgggaagacaatagcaggcatgctggggaagatctcctagtctaggctc |
| | gagttaagggcgaattcccgataaggatcttcctagagcatggctacgtagataagtagcatggcgggtt |
| | aatcattaactacaaggaacccctagtgatggagttggccactccctctctgcgcgctcgctcgctcactg |
| | aggccgggcgaccaaaggtcgcccgacgcccgggctttgcccgggcggcctcagtgagcgagcgag |
| | cgcg |
| 15 | TSL Sequence-Human PCDH15-CD1 full sequence (Retina isoform) |
| | 5′ITR, 3′ITR, CMV Promotor, PCDH15 DNA seq, SD sequence |
| | ctgcgcgctcgctcgctcactgaggccgcccgggcaaagcccgggcgtcgggcgacctttggtcgcc |
| | cggcctcagtgagcgagcgagcgcgcagagagggagtggccaactccatcactaggggttccttgtag |
| | ttaatgattaacccgccatgctacttatctacgtagccatgctctaggaagatcggaattcgcccttaagcta |
| | gcggcgcgccgtttaaaccgttacataacttacggtaaatggcccgcctggctgaccgcccaacgaccc |
| | ccgcccattgacgtcaataatgacgtatgttcccatagtaacgccaatagggactttccattgacgtcaatg |
| | ggtggagtatttacggtaaactgcccacttggcagtacatcaagtgtatcatatgccaagtccgccccctat |
| | tgacgtcaatgacggtaaatggcccgcctggcattatgcccagtacatgaccttacgggactttcctacttg |
| | gcagtacatctacgtattagtcatcgctattaccatggtgatgcggttttggcagtacaccaatgggcgtgg |
| | atagcggtttgactcacggggatttccaagtctccaccccattgacgtcaatgggagtttgttttggcacca |
| | aaatcaacgggactttccaaaatgtcgtaataaccccgccccgttgacgcaaatgggcggtaggcgtgta |
| | cggtgggaggtctatataagcagagctcgtttagtgaaccgtcagatcactagaagctttattgcggtagg |
| | ccgccaccatggatgtttcgacagttttatctctggacatgtttagcttcagggatcatcctgggctctctcttt |
| | gaaatctgcttgggccagtatgatgatgactggcaatatgaggattgcaaactagctaggggaggaccac |
| | cagctaccatagttgctattgatgaagaaagtcggaatggtacaattctggtggacaacatgctgatcaaa |
| | gggactgctggaggaccagaccccaccatagaactttctttaaaggataatgtggattactgggtgttgat |
| | ggatcctgttaagcaaatgcttttcctgaacagcaccggaagagttctggatagagatccaccgatgaaca |
| | tacactccattgtggtgcaggtccagtgcatcaacaaaaaagtgggcactattatctaccatgaagtgcga |
| | atagtggtgagagacaggaatgacaactcacccactttcaagcatgaaagctactatgccacagtgaatg |
| | agctcactccagttggtaccacaatattcacaggattttcaggagacaatggagctacagatatagatgat |
| | ggaccaaatggacagatagagtatgttattcagtataatccagatgatccgacatccaatgacacctttgaa |
| | attcccctaatgttgactggaaatatagtgttaaggaagaggctcaactatgaagataagactcgctactttg |
| | tcataatccaagctaatgaccgtgcccaaaatctgaatgagaggcgaaccaccaccaccactctcacagt |
| | ggatgttctggatggagatgacttgggtccaatgtttcttccttgtgtccttgtgccaaacactcgtgattgcc |
| | gtccactcacttatcaagctgccatacctgagttgagaactccggaagaactgaaccccattattgttacgc |
| | caccaatccaagccattgatcaggaccggaatattcaaccgccatcagataggccaggaatcctctattc |
| | catccttgttgggactcctgaggattacccacgatttttccatatgcatcctaggacagcagaacttagtctc |
| | ctggagccagtaaacagagactttcaccagaaatttgatttggttattaaggctgaacaagacaatggtcat |
| | cctcttcctgcctttgccggtctacacattgaaatactggatgaaaacaatcaaagtccatattttacaatgcc |
| | cagttatcaaggctatatcctggaatctgccccagtgggagcaaccatttcggacagtctcaatttgacttc |
| | acctttaagaatagtagctctggacaaggacatagaagatacaaaagacccagagcttcacctttttctga |
| | atgactacacctcagtcttcaccgtcacacagactggtattactcgctacctcaccttacttcaaccagtgga |
| | cagggaagaacagcaaacttacaccttttcgataacagcatttgatggtgtacaagaaagtgagccagtc |
| | atcgtcaatattcaagtgatggatgcaaatgataacacgccaaccttccctgaaatatcctatgatgtgtatg |
| | tttatacagacatgagacctggggacagtgtcatacagctcactgcagtcgacgcagacgaagggtcaa |
| | atggggagatcacatatgaaatccttgttggggctcagggagacttcatcatcaataaaacaacagggctt |
| | atcaccatcgctccaggggtggaaatgatagtcgggcggacttacgcactcacggtccaagcagcggat |
| | aatgctcctcctgcagagcgaaggaactccatctgcactgtgtatattgaagtgcttccaccaaataatcaa |
| | agccctcctcgcttcccacagctgatgtatagccttgaaattagtgaagccatgagggttggtgctgttttat |
| | taaatctacaggcaactgatcgagagggagactcaataacatatgccattgagaatggagatcctcagag |
| | agtttttaatctttcagaaaccacggggattctaaccttagggaaagcactggacagggaaagcactgatc |
| | gctacattctgatcatcacagcttcagatggcaggccagatgggacctcaactgccacagtaaacatagt |
| | ggtgacagatgtcaatgacaatgctccagtgtttgatccttatctgccaagaaatttatctgtggtaagtatca |
| | aggttacaagacaggtttaaggagaccaatagaaactgggcttgtcgagacagagaagactcttgcgttt |
| | ctgaggtaccgcatgcaccacggaccgggatcctagagcatggctaagatctcctatgctaggctcgag |
| | ttaagggcgaattcccgataaggatcttcctagagcatggctacgtagataagtagcatggcgggttaatc |
| | attaactacaaggaacccctagtgatggagttggccactccctctctgcgcgctcgctcgctcactgaggc |
| | cgggcgaccaaaggtcgcccgacgcccgggctttgcccgggcggcctcagtgagcgagcgagcgcg |
| | cag |
| 16 | TSR Sequence-Human PCDH15-CD1 full sequence (Retina isoform) |
| | 5′ITR, 3′ITR, SA site, PCDH15 DNA seq, bGH pA |
| | ctgcgcgctcgctcgctcactgaggccgcccgggcaaagcccgggcgtcgggcgacctttggtcgcc |
| | cggcctcagtgagcgagcgagcgcgcagagagggagtggccaactccatcactaggggttccttgtag |
| | ttaatgattaacccgccatgctacttatctacgtagccatgctctaggaagatcggaattcgcccttaagcta |
| | gcttaattaacctaggatcacgcatgctcgcgataggcacctattggtcttactgacatccactttgcctttct |
| | ctccacaggtggaagaagaagccaatgcctttgtgggtcaagtaaaagcaacagaccctgatgctggaa |
| | taaatggtcaagtgcactacagtttgggtaactttaataatctttttcgtatcacatccaatgggagcatttaca |
| | cagcagtgaagcttaacagagaagtcagggactactatgaacttgttgttgtggcaacagatggagcagt |
| | acaccctcgtcattcaactctaaccttggccatcaaggttttggacattgatgataacagtcctgtgttcacc |
| | aattcaacatacactgtccttgttgaagagaatttgccagctgggactaccatccttcaaatagaggccaaa |
| | gatgtcgaccttggagcaaatgtgtcttaccggataagaagcccagaagtgaagcacttttttgcactacat |
| | ccatttacaggagaactatcgcttttaaggagtttagattatgaggcatttccagaccaagaagcaagtatc |
| | acttttctggtagaggcctttgatatttatggaacaatgccacctggtattgctactgtcacagtgattgtaaa |
| | ggatatgaatgattatcctcctgtctttagtaaacgaatatacaaagggatggtggctccggatgcagtcaa |
| | gggtacacctatcacaacagtttatgctgaagatgcagaccctcctggattacctgcaagtcgtgtgaggt |
| | atagagtagatgatgtacagtttccttaccctgccagtatttttgaagtggaagaagattctggaagagtaat |
| | aacacgagtcaatcttaatgaagaacctacaacaatttttaagttggtggtggttgcttttgatgatggggag |
| | cctgtgatgtccagcagtgccacagtgaagattcttgtcttacatcctggtgagatcccacgcttcacacag |
| | gaggaatatagacctcctccagtaagtgaacttgccaccaaagggaccatggttggtgtaatttctgctgct |
| | gccattaatcaaagtattgtgtactccattgtttcaggaaatgaagaagatacatttggaattaataacatcac |
| | aggtgttatctatgtgaatggacctctggattatgagaccaggacaagctatgtacttcgagtccaagctga |
| | ttccctggaagtggtccttgccaatctccgagttccttcaaaaagcaatacagctaaagtatacattgagatt |
| | caggatgaaaataatcatcccccagtgtttcagaaaaaattctacatcggaggtgtatctgaagatgcaag |
| | aatgtttacttctgtactcagagtgaaggctactgataaagatactggcaattatagtgtcatggcctacaga |
| | ctcataataccaccaattaaagagggaaaagaaggatttgtagtggaaacatatacagggcttatcaaaac |
| | tgctatgctcttccataatatgaggagatcctacttcaagtttcaagttattgcaactgacgactatgggaag |
| | ggactgagcggcaaagccgatgtactcgtctccgtggtcaatcagctggatatgcaagtcattgtttccaa |
| | tgtgcctcctactctagtggaaaaaaagatagaagatcttacagagatcttggatcgctatgttcaggaaca |
| | aattcctggtgccaaggtcgtagtggagtccattggagctcgccggcatggagatgccttttccctagaag |
| | attacaccaaatgtgacttgactgtctatgcaattgacccccaaaccaacagagccatcgatagaaatgag |
| | ctttttaaatttttggatggcaaactacttgatatcaataaagactttcagccgtattatggggaaggaggacg |
| | cattctggagatccggactccagaggcagtgaccagcattaaaaagagaggagaaagtctaggataca |
| | cagaaggggccttgttggctctggccttcatcatcatcctctgctgcattcctgccatcttggtggttttggtc |
| | agctacagacagtttaaagtacgtcaagctgagtgtacaaagactgcacgaattcaggccgcattacccg |
| | cggctaaaccagcagtgccggctcctgcaccagtggcagcgcccccgccgccgccgccgcctccgcc |
| | aggtgcgcatctctatgaagaacttggagacagctcaatgcataatcttttccttctctaccattttcaacaaa |
| | gcaggggaaataactcagtctcagaagacaggaaacatcaacaagttgtgatgcccttttcttccaatact |
| | attgaggctcacaagtcagctcatgtagacggatcacttaagagcaacaaactgaagtctgcaagaaaat |
| | tcacatttctatctgatgaggatgacttaagtgcccataatcccctttataaggaaaacataagtcaagtatca |
| | acaaattcagacatttcacagagaacagattttgtagacccattttcacccaaaatacaagccaagagtaa |
| | gtctctgaggggcccaagagaaaagattcagaggctgtggagtcagtcagtcagcttacccaggaggct |
| | gatgaggaaagttccaaatagaccagagatcatagatctgcagcagtggcaaggcaccaggcagaaag |
| | ctgaaaatgaaaacactggaatctgtacaaacaaaagaggtagcagcaatccattgcttacaactgaaga |
| | ggcaaatttgacagagaaagaggaaataaggcaaggtgaaacactgatgatagaaggaacagaacagt |
| | tgaaatctctctcttcagactcttcattttgctttcccaggcctcacttctcattctccactttgccaactgtttca |
| | agaactgtggaactcaaatcagaacctaatgtcatcagttctcctgctgagtgttccttggaactttctccttc |
| | aaggccttgtgttttacattcttcactctctaggagagagacacctatttgtatgttacctattgaaaccgaaa |
| | gaaatatttttgaaaattttgcccatccaccaaacatctctccttctgcctgtccccttccccctcctcctcctat |
| | ttctcctccttctcctcctcctgctcctgctcctcttgctcctcctcctgacatttctcctttttctcttttttgtcct |
| | cctccctctcctccttctatccctcttcctcttcctcctcctacattttttccactttccgtttcaacgtctggtcccc |
| | caacaccacctcttctacctccatttccaactcctcttcctccaccacctccttctattccttgccctccacctc |
| | cttcagcttcatttctgtccacagagtgtgtctgtataacaggtgttaaatgcacgaccaacttgatgcctgc |
| | cgagaaaattaagtcctctatgacacagctatcaacaacgacagtgtgtaaaacagaccctcagagagaa |
| | ccaaaaggcatcctcagacacgttaaaaacttagcagaacttgaaaaatcagtagctaacatgtacagtca |
| | aatagaaaaaaactatctacgcacaaatgtttcagaacttcaaactatgtgcccttcagaagtaacaaatat |
| | ggaaatcacatctgaacaaaacaaggggagtttgaacaatattgtcgagggaactgaaaaacaatctcac |
| | agtcaatctacttcactgtaaactagtatttaaatcaattggcctcgactgtgccttctagttgccagccatctg |
| | ttgtttgcccctcccccgtgccttccttgaccctggaaggtgccactcccactgtcctttcctaataaaatga |
| | ggaaattgcatcgcattgtctgagtaggtgtcattctattctggggggtggggggggcaggacagcaag |
| | ggggaggattgggaagacaatagcaggcatgctggggaagatctcctagtctaggctcgagttaaggg |
| | cgaattcccgataaggatcttcctagagcatggctacgtagataagtagcatggcgggttaatcattaacta |
| | caaggaacccctagtgatggagttggccactccctctctgcgcgctcgctcgctcactgaggccgggcg |
| | accaaaggtcgcccgacgcccgggctttgcccgggcggcctcagtgagcgagcgagcgcgcag |
| 17 | HBL Sequence-Human PCDH15-CD2 full sequence (Inner ear isoform) |
| | 5′ITR, 3′ITR,CMV Promotor, SD site +5′ partial AP site, PCDH15 DNA |
| | seq |
| | ctgcgcgctcgctcgctcactgaggccgcccgggcaaagcccgggcgtcgggcgacctttggtcgcc |
| | cggcctcagtgagcgagcgagcgcgcagagagggagtggccaactccatcactaggggttccttgtag |
| | ttaatgattaacccgccatgctacttatctacgtagccatgctctaggaagatcggaattcgcccttaagcta |
| | gcggcgcgccgtttaaaccgttacataacttacggtaaatggcccgcctggctgaccgcccaacgaccc |
| | ccgcccattgacgtcaataatgacgtatgttcccatagtaacgccaatagggactttccattgacgtcaatg |
| | ggtggagtatttacggtaaactgcccacttggcagtacatcaagtgtatcatatgccaagtccgccccctat |
| | tgacgtcaatgacggtaaatggcccgcctggcattatgcccagtacatgaccttacgggactttcctacttg |
| | gcagtacatctacgtattagtcatcgctattaccatggtgatgcggttttggcagtacaccaatgggcgtgg |
| | atagcggtttgactcacggggatttccaagtctccaccccattgacgtcaatgggagtttgttttggcacca |
| | aaatcaacgggactttccaaaatgtcgtaataaccccgccccgttgacgcaaatgggcggtaggcgtgta |
| | cggtgggaggtctatataagcagagctcgtttagtgaaccgtcagatcactagaagctttattgcggtagg |
| | ccgccaccatggatgtttcgacagttttatctctggacatgtttagcttcagggatcatcctgggctctctcttt |
| | gaaatctgcttgggccagtatgatgatgactggcaatatgaggattgcaaactagctaggggaggaccac |
| | cagctaccatagttgctattgatgaagaaagtcggaatggtacaattctggtggacaacatgctgatcaaa |
| | gggactgctggaggaccagaccccaccatagaactttctttaaaggataatgtggattactgggtgttgat |
| | ggatcctgttaagcaaatgcttttcctgaacagcaccggaagagttctggatagagatccaccgatgaaca |
| | tacactccattgtggtgcaggtccagtgcatcaacaaaaaagtgggcactattatctaccatgaagtgcga |
| | atagtggtgagagacaggaatgacaactcacccactttcaagcatgaaagctactatgccacagtgaatg |
| | agctcactccagttggtaccacaatattcacaggattttcaggagacaatggagctacagatatagatgat |
| | ggaccaaatggacagatagagtatgttattcagtataatccagatgatccgacatccaatgacacctttgaa |
| | attcccctaatgttgactggaaatatagtgttaaggaagaggctcaactatgaagataagactcgctactttg |
| | tcataatccaagctaatgaccgtgcccaaaatctgaatgagaggcgaaccaccaccaccactctcacagt |
| | ggatgttctggatggagatgacttgggtccaatgtttcttccttgtgtccttgtgccaaacactcgtgattgcc |
| | gtccactcacttatcaagctgccatacctgagttgagaactccggaagaactgaaccccattattgttacgc |
| | caccaatccaagccattgatcaggaccggaatattcaaccgccatcagataggccaggaatcctctattc |
| | catccttgttgggactcctgaggattacccacgatttttccatatgcatcctaggacagcagaacttagtctc |
| | ctggagccagtaaacagagactttcaccagaaatttgatttggttattaaggctgaacaagacaatggtcat |
| | cctcttcctgcctttgccggtctacacattgaaatactggatgaaaacaatcaaagtccatattttacaatgcc |
| | cagttatcaaggctatatcctggaatctgccccagtgggagcaaccatttcggacagtctcaatttgacttc |
| | acctttaagaatagtagctctggacaaggacatagaagatacaaaagacccagagcttcacctttttctga |
| | atgactacacctcagtcttcaccgtcacacagactggtattactcgctacctcaccttacttcaaccagtgga |
| | cagggaagaacagcaaacttacaccttttcgataacagcatttgatggtgtacaagaaagtgagccagtc |
| | atcgtcaatattcaagtgatggatgcaaatgataacacgccaaccttccctgaaatatcctatgatgtgtatg |
| | tttatacagacatgagacctggggacagtgtcatacagctcactgcagtcgacgcagacgaagggtcaa |
| | atggggagatcacatatgaaatccttgttggggctcagggagacttcatcatcaataaaacaacagggctt |
| | atcaccatcgctccaggggtggaaatgatagtcgggcggacttacgcactcacggtccaagcagcggat |
| | aatgctcctcctgcagagcgaaggaactccatctgcactgtgtatattgaagtgcttccaccaaataatcaa |
| | agccctcctcgcttcccacagctgatgtatagccttgaaattagtgaagccatgagggttggtgctgttttat |
| | taaatctacaggcaactgatcgagagggagactcaataacatatgccattgagaatggagatcctcagag |
| | agtttttaatctttcagaaaccacggggattctaaccttagggaaagcactggacagggaaagcactgatc |
| | gctacattctgatcatcacagcttcagatggcaggccagatgggacctcaactgccacagtaaacatagt |
| | ggtgacagatgtcaatgacaatgctccagtgtttgatccttatctgccaagaaatttatctgtggtaagtatca |
| | aggttacaagacaggtttaaggagaccaatagaaactgggcttgtcgagacagagaagactcttgcgttt |
| | ctgaggtaccgcatgcaccacggaccgtcgacgcagggcagcctctgtcatctccatcagggaggggt |
| | ccagtgtggagtctcggtggatctcgtatttcatgtctccaggctcaaagagacccatgagatgggtcaca |
| | gacgggtccagggaagcatgcatgagctcagtgcggttccacacataccgggcaccctggcgcttcgc |
| | cagccattcctgcaccagattcttcccgtccagcctggtcccaccttggctgtagtcatctgggtactcagg |
| | gtctggggttcccatgcgaaacatgtactttcggcctccaggatcctagagcatggctaagatctcctatgc |
| | taggctcgagttaagggcgaattcccgataaggatcttcctagagcatggctacgtagataagtagcatg |
| | gcgggttaatcattaactacaaggaacccctagtgatggagttggccactccctctctgcgcgctcgctcg |
| | ctcactgaggccgggcgaccaaaggtcgcccgacgcccgggctttgcccgggcggcctcagtgagcg |
| | agcgagcgcg |
| 18 | HBR Sequence-Human PCDH15-CD2 full sequence (Inner ear isoform) |
| | 5′ITR, 3′ITR, SA site +3′ partial AP site, PCDH15 DNA seq, bGH pA |
| | ctgcgcgctcgctcgctcactgaggccgcccgggcaaagcccgggcgtcggggacctttggtcgcc |
| | cggcctcagtgagcgagcgagcgcgcagagagggagtggccaactccatcactaggggttccttgtag |
| | ttaatgattaacccgccatgctacttatctacgtagccatgctctaggaagatcggaattcgcccttaagcta |
| | gcttaattaacgcagggcagcctctgtcatctccatcagggaggggtccagtgtggagtctcggtggatct |
| | cgtatttcatgtctccaggctcaaagagacccatgagatgggtcacagacgggtccagggaagcatgca |
| | tgagctcagtgcggttccacacataccgggcaccctggcgcttcgccagccattcctgcaccagattcttc |
| | ccgtccagcctggtcccaccttggctgtagtcatctgggtactcagggtctggggttcccatgcgaaacat |
| | gtactttcggcctccaggacctaggatcacgcatgctcgcgataggcacctattggtcttactgacatcca |
| | ctttgcctttctctccacaggtggaagaagaagccaatgcctttgtgggtcaagtaaaagcaacagaccct |
| | gatgctggaataaatggtcaagtgcactacagtttgggtaactttaataatctttttcgtatcacatccaatgg |
| | gagcatttacacagcagtgaagcttaacagagaagtcagggactactatgaacttgttgttgtggcaacag |
| | atggagcagtacaccctcgtcattcaactctaaccttggccatcaaggttttggacattgatgataacagtc |
| | ctgtgttcaccaattcaacatacactgtccttgttgaagagaatttgccagctgggactaccatccttcaaat |
| | agaggccaaagatgtcgaccttggagcaaatgtgtcttaccggataagaagcccagaagtgaagcacttt |
| | tttgcactacatccatttacaggagaactatcgcttttaaggagtttagattatgaggcatttccagaccaaga |
| | agcaagtatcacttttctggtagaggcctttgatatttatggaacaatgccacctggtattgctactgtcacag |
| | tgattgtaaaggatatgaatgattatcctcctgtctttagtaaacgaatatacaaagggatggtggctccgga |
| | tgcagtcaagggtacacctatcacaacagtttatgctgaagatgcagaccctcctggattacctgcaagtc |
| | gtgtgaggtatagagtagatgatgtacagtttccttaccctgccagtatttttgaagtggaagaagattctgg |
| | aagagtaataacacgagtcaatcttaatgaagaacctacaacaatttttaagttggtggtggttgcttttgatg |
| | atggggagcctgtgatgtccagcagtgccacagtgaagattcttgtcttacatcctggtgagatcccacgc |
| | ttcacacaggaggaatatagacctcctccagtaagtgaacttgccaccaaagggaccatggttggtgtaat |
| | ttctgctgctgccattaatcaaagtattgtgtactccattgtttcaggaaatgaagaagatacatttggaattaa |
| | taacatcacaggtgttatctatgtgaatggacctctggattatgagaccaggacaagctatgtacttcgagt |
| | ccaagctgattccctggaagtggtccttgccaatctccgagttccttcaaaaagcaatacagctaaagtata |
| | cattgagattcaggatgaaaataatcatcccccagtgtttcagaaaaaattctacatcggaggtgtatctga |
| | agatgcaagaatgtttacttctgtactcagagtgaaggctactgataaagatactggcaattatagtgtcatg |
| | gcctacagactcataataccaccaattaaagagggaaaagaaggatttgtagtggaaacatatacagggc |
| | ttatcaaaactgctatgctcttccataatatgaggagatcctacttcaagtttcaagttattgcaactgacgact |
| | atgggaagggactgagcggcaaagccgatgtactcgtctccgtggtcaatcagctggatatgcaagtcat |
| | tgtttccaatgtgcctcctactctagtggaaaaaaagatagaagatcttacagagatcttggatcgctatgtt |
| | caggaacaaattcctggtgccaaggtcgtagtggagtccattggagctcgccggcatggagatgcctttt |
| | ccctagaagattacaccaaatgtgacttgactgtctatgcaattgacccccaaaccaacagagccatcgat |
| | agaaatgagctttttaaatttttggatggcaaactacttgatatcaataaagactttcagccgtattatgggg |
| | aaggaggacgcattctggagatccggactccagaggcagtgaccagcattaaaaagagaggagaaag |
| | tctaggatacacagaaggggccttgttggctctggccttcatcatcatcctctgctgcattcctgccatcttg |
| | gtggttttggtcagctacagacagtttaaagtacgtcaagctgagtgtacaaagactgcacgaattcagg |
| | ccgcattacccgcggctaaaccagcagtgccggctcctgcaccagtggcagcgcccccgccgccgcc |
| | gccgcctccgccaggtgcgcatctctatgaagaacttggagacagctcaatgcataagtatgaaatgcct |
| | caatatgggagtcgccgtcgattgttaccaccagctggacaggaggaatatggtgaggtggttggtgaa |
| | gctgaggaagaatatgaggaggaagaggaagagccaaagaaaattaaaaaaccaaaggttgaaattag |
| | agagcctagtgaggaggaagaagtagttgtaactatcgaaaaaccaccagcagctgagcctacatacac |
| | aacatggaagagagccagaatattccccatgatttttaagaaagttagaggattagctgataaaagaggaa |
| | tcgttgaccttgagggtgaagagtggcagagacgccttgaggaagaagataaagattatttgaaactcac |
| | tctggaccaagaggaagcaacagaaagcactgtagaatcagaggaggaatcctccagcgactatactg |
| | aatacagtgaagaagagtctgagttcagtgagtctgagactacagaagaggaatctgagtcagagacac |
| | cctctgaggaggaggagagttccacccctgaatcagaagaatcggaatccacagagtcagaaggagaa |
| | aaagcaaggaaaaacattgtgcttgcaagaagaaggcccatggttgaggaggtcaaggaagtcaaggg |
| | taggaaagaggagccacaagaagaacaaaaagaacctaagatggaagaagaagaacactcagaaga |
| | agaagaaagtggaccagcccctgtggaagaaagtacagaccctgaagctcaagatatccctgaagagg |
| | gcagtgcagaatcagcttcggtggaaggaggtgtggaaagtgaggaggaatcagaatcaggtagtagt |
| | agcagtagtagcgaaagtcagtctggaggtccatggggctatcaggtaccagcgtatgacagaagcaa |
| | gaatgcaaaccaaaagaagtcgccaggagcaaactctgaaggttacaacacagcactttgatatttaaat |
| | caattggcctcgactgtgccttctagttgccagccatctgttgtttgcccctcccccgtgccttccttgaccct |
| | ggaaggtgccactcccactgtcctttcctaataaaatgaggaaattgcatcgcattgtctgagtaggtgtca |
| | ttctattctggggggtggggggggcaggacagcaagggggaggattgggaagacaatagcaggcat |
| | gctggggaagatctcctagtctaggctcgagttaagggcgaattcccgataaggatcttcctagagcatg |
| | gctacgtagataagtagcatggcgggttaatcattaactacaaggaacccctagtgatggagttggccact |
| | ccctctctgcgcgctcgctcgctcactgaggccgggcgaccaaaggtcgcccgacgcccgggctttgc |
| | ccgggcggcctcagtgagcgagcgagcgcg |
| 19 | TSL Sequence-Human PCDH15-CD2 full sequence (Inner ear isoform) |
| | 5′ITR, 3′ITR, CMV Promotor, PCDH15 DNA seq, SD sequence |
| | ctgcgcgctcgctcgctcactgaggccgcccgggcaaagcccgggcgtcgggcgacctttggtcgcc |
| | cggcctcagtgagcgagcgagcgcgcagagagggagtggccaactccatcactaggggttccttgtag |
| | ttaatgattaacccgccatgctacttatctacgtagccatgctctaggaagatcggaattcgcccttaagcta |
| | gcggcgcgccgtttaaaccgttacataacttacggtaaatggcccgcctggctgaccgcccaacgaccc |
| | ccgcccattgacgtcaataatgacgtatgttcccatagtaacgccaatagggactttccattgacgtcaatg |
| | ggtggagtatttacggtaaactgcccacttggcagtacatcaagtgtatcatatgccaagtccgccccctat |
| | tgacgtcaatgacggtaaatggcccgcctggcattatgcccagtacatgaccttacgggactttcctacttg |
| | gcagtacatctacgtattagtcatcgctattaccatggtgatgcggttttggcagtacaccaatgggcgtgg |
| | atagcggtttgactcacggggatttccaagtctccaccccattgacgtcaatgggagtttgttttggcacca |
| | aaatcaacgggactttccaaaatgtcgtaataaccccgccccgttgacgcaaatgggcggtaggcgtgta |
| | cggtgggaggtctatataagcagagctcgtttagtgaaccgtcagatcactagaagctttattgcggtagg |
| | ccgccaccatggatgtttcgacagttttatctctggacatgtttagcttcagggatcatcctgggctctctcttt |
| | gaaatctgcttgggccagtatgatgatgactggcaatatgaggattgcaaactagctaggggaggaccac |
| | cagctaccatagttgctattgatgaagaaagtcggaatggtacaattctggtggacaacatgctgatcaaa |
| | gggactgctggaggaccagaccccaccatagaactttctttaaaggataatgtggattactgggtgttgat |
| | ggatcctgttaagcaaatgcttttcctgaacagcaccggaagagttctggatagagatccaccgatgaaca |
| | tacactccattgtggtgcaggtccagtgcatcaacaaaaaagtgggcactattatctaccatgaagtgcga |
| | atagtggtgagagacaggaatgacaactcacccactttcaagcatgaaagctactatgccacagtgaatg |
| | agctcactccagttggtaccacaatattcacaggattttcaggagacaatggagctacagatatagatgat |
| | ggaccaaatggacagatagagtatgttattcagtataatccagatgatccgacatccaatgacacctttgaa |
| | attcccctaatgttgactggaaatatagtgttaaggaagaggctcaactatgaagataagactcgctactttg |
| | tcataatccaagctaatgaccgtgcccaaaatctgaatgagaggcgaaccaccaccaccactctcacagt |
| | ggatgttctggatggagatgacttgggtccaatgtttcttccttgtgtccttgtgccaaacactcgtgattgcc |
| | gtccactcacttatcaagctgccatacctgagttgagaactccggaagaactgaaccccattattgttacgc |
| | caccaatccaagccattgatcaggaccggaatattcaaccgccatcagataggccaggaatcctctattc |
| | catccttgttgggactcctgaggattacccacgatttttccatatgcatcctaggacagcagaacttagtctc |
| | ctggagccagtaaacagagactttcaccagaaatttgatttggttattaaggctgaacaagacaatggtcat |
| | cctcttcctgcctttgccggtctacacattgaaatactggatgaaaacaatcaaagtccatattttacaatgcc |
| | cagttatcaaggctatatcctggaatctgccccagtgggagcaaccatttcggacagtctcaatttgacttc |
| | acctttaagaatagtagctctggacaaggacatagaagatacaaaagacccagagcttcacctttttctga |
| | atgactacacctcagtcttcaccgtcacacagactggtattactcgctacctcaccttacttcaaccagtgga |
| | cagggaagaacagcaaacttacaccttttcgataacagcatttgatggtgtacaagaaagtgagccagtc |
| | atcgtcaatattcaagtgatggatgcaaatgataacacgccaaccttccctgaaatatcctatgatgtgtatg |
| | tttatacagacatgagacctggggacagtgtcatacagctcactgcagtcgacgcagacgaagggtcaa |
| | atggggagatcacatatgaaatccttgttggggctcagggagacttcatcatcaataaaacaacagggctt |
| | atcaccatcgctccaggggtggaaatgatagtcgggcggacttacgcactcacggtccaagcagcggat |
| | aatgctcctcctgcagagcgaaggaactccatctgcactgtgtatattgaagtgcttccaccaaataatcaa |
| | agccctcctcgcttcccacagctgatgtatagccttgaaattagtgaagccatgagggttggtgctgttttat |
| | taaatctacaggcaactgatcgagagggagactcaataacatatgccattgagaatggagatcctcagag |
| | agtttttaatctttcagaaaccacggggattctaaccttagggaaagcactggacagggaaagcactgatc |
| | gctacattctgatcatcacagcttcagatggcaggccagatgggacctcaactgccacagtaaacatagt |
| | ggtgacagatgtcaatgacaatgctccagtgtttgatccttatctgccaagaaatttatctgtggtaagtatca |
| | aggttacaagacaggtttaaggagaccaatagaaactgggcttgtcgagacagagaagactcttgcgttt |
| | ctgaggtaccgcatgcaccacggaccgggatcctagagcatggctaagatctcctatgctaggctcgag |
| | ttaagggcgaattcccgataaggatcttcctagagcatggctacgtagataagtagcatggcgggttaatc |
| | attaactacaaggaacccctagtgatggagttggccactccctctctgcgcgctcgctcgctcactgaggc |
| | cgggcgaccaaaggtcgcccgacgcccgggctttgcccgggcggcctcagtgagcgagcgagcgcg |
| | cag |
| 20 | TSR Sequence-Human PCDH15-CD2 full sequence (Inner ear isoform) |
| | 5′ITR, 3′ITR,SA site, PCDH15 DNA seq, bGH pA |
| | ctgcgcgctcgctcgctcactgaggccgcccgggcaaagcccgggcgtcgggcgacctttggtcgcc |
| | cggcctcagtgagcgagcgagcgcgcagagagggagtggccaactccatcactaggggttccttgtag |
| | ttaatgattaacccgccatgctacttatctacgtagccatgctctaggaagatcggaattcgcccttaagcta |
| | gcttaattaacctaggatcacgcatgctcgcgataggcacctattggtcttactgacatccactttgcctttct |
| | ctccacaggtggaagaagaagccaatgcctttgtgggtcaagtaaaagcaacagaccctgatgctggaa |
| | taaatggtcaagtgcactacagtttgggtaactttaataatctttttcgtatcacatccaatgggagcatttaca |
| | cagcagtgaagcttaacagagaagtcagggactactatgaacttgttgttgtggcaacagatggagcagt |
| | acaccctcgtcattcaactctaaccttggccatcaaggttttggacattgatgataacagtcctgtgttcacc |
| | aattcaacatacactgtccttgttgaagagaatttgccagctgggactaccatccttcaaatagaggccaaa |
| | gatgtcgaccttggagcaaatgtgtcttaccggataagaagcccagaagtgaagcacttttttgcactacat |
| | ccatttacaggagaactatcgcttttaaggagtttagattatgaggcatttccagaccaagaagcaagtatc |
| | acttttctggtagaggcctttgatatttatggaacaatgccacctggtattgctactgtcacagtgattgtaaa |
| | ggatatgaatgattatcctcctgtctttagtaaacgaatatacaaagggatggtggctccggatgcagtcaa |
| | gggtacacctatcacaacagtttatgctgaagatgcagaccctcctggattacctgcaagtcgtgtgaggt |
| | atagagtagatgatgtacagtttccttaccctgccagtatttttgaagtggaagaagattctggaagagtaat |
| | aacacgagtcaatcttaatgaagaacctacaacaatttttaagttggtggtggttgcttttgatgatggggag |
| | cctgtgatgtccagcagtgccacagtgaagattcttgtcttacatcctggtgagatcccacgcttcacacag |
| | gaggaatatagacctcctccagtaagtgaacttgccaccaaagggaccatggttggtgtaatttctgctgct |
| | gccattaatcaaagtattgtgtactccattgtttcaggaaatgaagaagatacatttggaattaataacatcac |
| | aggtgttatctatgtgaatggacctctggattatgagaccaggacaagctatgtacttcgagtccaagctga |
| | ttccctggaagtggtccttgccaatctccgagttccttcaaaaagcaatacagctaaagtatacattgagatt |
| | caggatgaaaataatcatcccccagtgtttcagaaaaaattctacatcggaggtgtatctgaagatgcaag |
| | aatgtttacttctgtactcagagtgaaggctactgataaagatactggcaattatagtgtcatggcctacaga |
| | ctcataataccaccaattaaagagggaaaagaaggatttgtagtggaaacatatacagggcttatcaaaac |
| | tgctatgctcttccataatatgaggagatcctacttcaagtttcaagttattgcaactgacgactatgggaag |
| | ggactgagcggcaaagccgatgtactcgtctccgtggtcaatcagctggatatgcaagtcattgtttccaa |
| | tgtgcctcctactctagtggaaaaaaagatagaagatcttacagagatcttggatcgctatgttcaggaaca |
| | aattcctggtgccaaggtcgtagtggagtccattggagctcgccggcatggagatgccttttccctagaag |
| | attacaccaaatgtgacttgactgtctatgcaattgacccccaaaccaacagagccatcgatagaaatgag |
| | ctttttaaatttttggatggcaaactacttgatatcaataaagactttcagccgtattatggggaaggaggacg |
| | cattctggagatccggactccagaggcagtgaccagcattaaaaagagaggagaaagtctaggataca |
| | cagaaggggccttgttggctctggccttcatcatcatcctctgctgcattcctgccatcttggtggttttggtc |
| | agctacagacagtttaaagtacgtcaagctgagtgtacaaagactgcacgaattcaggccgcattacccg |
| | cggctaaaccagcagtgccggctcctgcaccagtggcagcgcccccgccgccgccgccgcctccgcc |
| | aggtgcgcatctctatgaagaacttggagacagctcaatgcataagtatgaaatgcctcaatatgggagtc |
| | gccgtcgattgttaccaccagctggacaggaggaatatggtgaggtggttggtgaagctgaggaagaat |
| | atgaggaggaagaggaagagccaaagaaaattaaaaaaccaaaggttgaaattagagagcctagtgag |
| | gaggaagaagtagttgtaactatcgaaaaaccaccagcagctgagcctacatacacaacatggaagag |
| | agccagaatattccccatgatttttaagaaagttagaggattagctgataaaagaggaatcgttgaccttga |
| | gggtgaagagtggcagagacgccttgaggaagaagataaagattatttgaaactcactctggaccaaga |
| | ggaagcaacagaaagcactgtagaatcagaggaggaatcctccagcgactatactgaatacagtgaag |
| | aagagtctgagttcagtgagtctgagactacagaagaggaatctgagtcagagacaccctctgaggagg |
| | aggagagttccacccctgaatcagaagaatcggaatccacagagtcagaaggagaaaaagcaaggaa |
| | aaacattgtgcttgcaagaagaaggcccatggttgaggaggtcaaggaagtcaagggtaggaaagagg |
| | agccacaagaagaacaaaaagaacctaagatggaagaagaagaacactcagaagaagaagaaagtg |
| | gaccagcccctgtggaagaaagtacagaccctgaagctcaagatatccctgaagagggcagtgcagaa |
| | tcagcttcggtggaaggaggtgtggaaagtgaggaggaatcagaatcaggtagtagtagcagtagtagc |
| | gaaagtcagtctggaggtccatggggctatcaggtaccagcgtatgacagaagcaagaatgcaaacca |
| | aaagaagtcgccaggagcaaactctgaaggttacaacacagcactttgaatttaaatcaattggcctcgac |
| | tgtgccttctagttgccagccatctgttgtttgcccctcccccgtgccttccttgaccctggaaggtgccact |
| | cccactgtcctttcctaataaaatgaggaaattgcatcgcattgtctgagtaggtgtcattctattctggggg |
| | gtggggtggggcaggacagcaagggggaggattgggaagacaatagcaggcatgctggggaagatc |
| | tcctagtctaggctcgagttaagggcgaattcccgataaggatcttcctagagcatggctacgtagataag |
| | tagcatggcgggttaatcattaactacaaggaacccctagtgatggagttggccactccctctctgcgcgc |
| | tcgctcgctcactgaggccgggcgaccaaaggtcgcccgacgcccgggctttgcccgggggcctca |
| | gtgagcgagcgagcgcgcag |

In some embodiments, the invention provides a vector comprising a polynucleotide sequence of SEQ ID NO:13 or a variant thereof comprising at least 60% identity (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) thereto.

In another aspect, the invention provides a vector comprising a polynucleotide sequence of SEQ ID NO:14 or a variant thereof comprising at least 60% identity (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) thereto.

In another aspect, the invention provides a vector comprising a polynucleotide sequence of SEQ ID NO:15 or a variant thereof comprising at least 60% identity (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) thereto.

In another aspect, the invention provides a vector comprising a polynucleotide sequence of SEQ ID NO:16 or a variant thereof comprising at least 60% identity (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) thereto.

In another aspect, the invention provides a vector comprising a polynucleotide sequence of SEQ ID NO:17 or a variant thereof comprising at least 60% identity (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) thereto.

In another aspect, the invention provides a vector comprising a polynucleotide sequence of SEQ ID NO:18 or a variant thereof comprising at least 60% identity (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) thereto.

In another aspect, the invention provides a vector comprising a polynucleotide sequence of SEQ ID NO:19 or a variant thereof comprising at least 60% identity (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) thereto.

In another aspect, the invention provides a vector comprising a polynucleotide sequence of SEQ ID NO:20 or a variant thereof comprising at least 60% identity (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) thereto.

In some embodiments, the dual vector system comprises a first vector that comprises a nucleotide sequence that is at least 60% identical (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) to SEQ ID NO:13 and the second vector comprises a nucleotide sequence that is at least 60% identical (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) to SEQ ID NO:14.

In some embodiments, the dual vector system comprises a first vector that comprises a nucleotide sequence that is at least 60% identical (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) to SEQ ID NO:15, and the second vector comprises a nucleotide sequence that is at least 60% identical (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) to SEQ ID NO:16.

In some embodiments, the dual vector system comprises a first vector that comprises a nucleotide sequence that is at least 60% identical (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) to SEQ ID NO: 17 and the second vector comprises a nucleotide sequence that is at least 60% identical (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) to SEQ ID NO:18.

In some embodiments, the dual vector system comprises a first vector that comprises a nucleotide sequence that is at least 60% identical (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) to SEQ ID NO: 19 and the second vector comprises a nucleotide sequence that is at least 60% identical (at least 60%, 65%, 70%, 75%, 80%, 85%, 86%, 87%, 88%, 89%, 90%, 91%, 92%, 93%, 94%, 95%, 96%, 97%, 98%, 99% or greater sequence identity) to SEQ ID NO:20.

### Pharmaceutical Compositions

In another embodiment, the invention provides a pharmaceutical composition comprising an effective amount of a vector as provided herein in combination with a pharmaceutically acceptable excipient.

In another embodiment, the invention provides a pharmaceutical compositions comprising dual vector systems as provided herein. In some embodiments, the compositions can comprise effective amounts of the first or second vector or both in combination with a pharmaceutically acceptable excipient.

A “pharmaceutically acceptable excipient” is a material that acts in concert with an active ingredient of a medication to impart desirable qualities to a drug intended to be introduced into the body of a subject. The desirable qualities could include enhancing long term stability, acting as a diluent for an active ingredient that must be administered in small amounts, enhancement of therapeutic qualities of an active ingredient, facilitating absorption of an active ingredient into the body, adjusting viscosity, enhancing solubility of an active ingredient, or modifying macroscopic properties of a drug such as flowability or adhesion. Pharmaceutically acceptable excipients can comprise but are not limited to diluents, binders, pH stabilizing agents, disintegrants, surfactants, glidants, dyes, flavoring agents, preservatives, sorbents, sweeteners and lubricants. These materials can take many different forms. See, e.g., Nema, et al., Excipients and their use in injectable products, PDA J. Pharm. Sci. & Tech. 1997, 51 (4): 166-171.

The nucleic acid vectors (e.g., AAV vectors) described herein may be incorporated into a vehicle for administration into a patient, such as a human patient suffering from any of the conditions described herein. Pharmaceutical compositions containing vectors, such as viral vectors, that contain a polynucleotide encoding a portion of an Pcdh15 can be prepared using methods known in the art. For example, such compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions.

Mixtures of the nucleic acid vectors (e.g., AAV vectors) described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the inner ear, the composition may be formulated to contain a synthetic perilymph solution. An exemplary synthetic perilymph solution includes 20-200 mM NaCl, 1-5 mM KCl, 0.1-10 mM CaCl₂, 1-10 mM glucose, and 2-50 mM FIEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

The compositions described herein may be administered to a subject with sensorineural hearing loss, vision loss, or vestibular dysfunction by a variety of routes, such as local administration to the inner ear (e.g., administration into the perilymph or endolymph, e.g., by injection or catheter insertion through the round window membrane, injection into a semicircular canal, by canalostomy, or by intratympanic or transtympanic injection, e.g., administration to a cochlear hair cell), retina, intravenous, parenteral, intradermal, transdermal, intramuscular, intranasal, subcutaneous, percutaneous, intratracheal, intraperitoneal, intraarterial, intravascular, inhalation, perfusion, lavage, and oral administration. If the compositions are administered by direct delivery to the inner ear, a second fenestration or vent hole may be added elsewhere in the inner ear. The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bimonthly, monthly, or bi-weekly). In some embodiments, the first and second nucleic acid vectors (e.g., AAV vectors) are administered simultaneously (e.g., in one composition). In some embodiments, the first and second nucleic acid vectors (e.g., AAV vectors) are administered sequentially (e.g., the second nucleic acid vector is administered immediately after the first nucleic acid vector, or 5 minutes, 10 minutes, 15 minutes, 20 minutes, 25 minutes, 30 minutes, 45 minutes, 1 hour, 2 hours, 8 hours, 12 hours, 1 day, 2 days, 7 days, two weeks, 1 month or more after the first nucleic acid vector). The first and second nucleic acid vector can have the same serotype or different serotypes (e.g., AAV serotypes).

Subjects that may be treated as described herein are subjects having or at risk of developing sensorineural hearing loss, vision loss or vestibular dysfunction. The compositions and methods described herein can be used to treat subjects having a mutation in Pcdh15 (e.g., a mutation that reduces Pcdh15 function or expression, or a Pcdh15 mutation associated with sensorineural hearing loss), subjects having a family history of autosomal recessive sensorineural hearing loss, vision loss or vestibular dysfunction, or subjects whose Pcdh15 mutational status and/or Pcdh15 activity level is unknown. The methods described herein may include a step of screening a subject for a mutation in Pcdh15 prior to treatment with or administration of the compositions described herein. A subject can be screened for an Pcdh15 mutation using standard methods known to those of skill in the art (e.g., genetic testing). The methods described herein may also include a step of assessing hearing, vision or vestibular dysfunction in a subject prior to treatment with or administration of the compositions described herein. Hearing can be assessed using standard tests, such as audiometry, ABR, electrocochleography (ECOG), and otoacoustic emissions.

The compositions and methods described herein may also be administered as a preventative treatment to patients at risk of developing hearing loss, vision loss or vestibular dysfunction, e.g., patients who have a family history of such conditions or patients carrying a Pcdh15 mutation who do not yet exhibit the condition.

Treatment may include administration of a composition containing the nucleic acid vectors (e.g., AAV vectors) described herein in various unit doses. Each unit dose will ordinarily contain a predetermined quantity of the therapeutic composition. The quantity to be administered, and the particular route of administration and formulation, are within the skill of those in the clinical arts. A unit dose need not be administered as a single injection but may include continuous infusion over a set period of time. Dosing may be performed using a syringe pump to control infusion rate in order to minimize damage to the tissue administered.

In cases in which the nucleic acid vectors are AAV vectors (e.g., AAV1, AAV2, AAV2quad (Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, or PHP.S vectors), the AAV vectors may have a titer of, for example, from about 1×10⁹ vector genomes (VG)/mL to about 1×10¹⁶ VG/mL (e.g., 1×10⁹ VG/mL, 2×10⁹ VG/mL, 3×10⁹ VG/mL, 4×10⁹ VG/mL, 5×10⁹ VG/mL, 6×10⁹ VG/mL, 7×10⁹ VG/mL, 8×10⁹ VG/mL, 9×10⁹ VG/mL, 1×10¹⁰ VG/mL, 2×10¹⁰ VG/mL, 3×10¹⁰ VG/mL, 4×10¹⁰ VG/mL, 5×10¹⁰ VG/mL, 6×10¹⁰ VG/mL, 7×10¹⁰ VG/mL, 8×10¹⁰ VG/mL, 9×10¹⁰ VG/mL, 1×10¹¹ VG/mL, 2×10¹¹ VG/mL, 3×10¹¹ VG/mL, 4×10¹¹ VG/mL, 5×10¹¹ VG/mL, 6×10¹¹ VG/mL, 7×10¹¹ VG/mL, 8×10¹¹ VG/mL, 9×10¹¹ VG/mL, 1×10¹² VG/mL, 2×10¹² VG/mL, 3×10¹² VG/mL, 4×10¹² VG/mL, 5×10¹² VG/mL, 6×10¹² VG/mL, 7×10¹² VG/mL, 8×10¹² VG/mL, 9×10¹² VG/mL, 1×10¹³ VG/mL, 2×10¹³ VG/mL, 3×10¹³ VG/mL, 4×10¹³ VG/mL, 5×10¹³ VG/mL, 6×10¹³ VG/mL, 7×10¹³ VG/mL, 8×10¹³ VG/mL, 9×10¹³ VG/mL, 1×10¹⁴ VG/mL, 2×10¹⁴ VG/mL, 3×10¹⁴ VG/mL, 4×10¹⁴ VG/mL, 5×10¹⁴ VG/mL, 6×10¹⁴ VG/mL, 7×10¹⁴ VG/mL, 8×10^{14 VG/mL,} 9×10¹⁴ VG/mL, 1×10¹⁵ VG/mL, 2×10¹⁵ VG/mL, 3×10¹⁵ VG/mL, 4×10¹⁵ VG/mL, 5×10¹⁵ VG/mL, 6×10¹⁵ VG/mL, 7×10¹⁵ VG/mL, 8×10¹⁵ VG/mL, 9×10¹⁵ VG/mL, or 1×10¹⁶ VG/mL) in a volume of, e.g., about 1 pL to about 200 pL (e.g., 1, 2, 3, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 pL).

In some embodiments, the AAV vectors may be administered to the subject at a dose of about 1×10⁷ VG/ear to about 2×10¹⁵ VG/ear (e.g., 1×10⁷ VG/ear, 2×10⁷ VG/ear, 3×10⁷ VG/ear, 4×10⁷ VG/ear, 5×10⁷ VG/ear, 6×10⁷ VG/ear, 7×10⁷ VG/ear, 8×10⁷ VG/ear, 9×10⁷ VG/ear, 1×10⁸ VG/ear, 2×10⁸ VG/ear, 3×10⁸ VG/ear, 4×10⁸ VG/ear, 5×10⁸ VG/ear, 6×10⁸ VG/ear, 7×10⁸ VG/ear, 8×10⁸ VG/ear, 9×10⁸ VG/ear, 1×10⁹ VG/ear, 2×10⁹ VG/ear, 3×10⁹ VG/ear, 4×10⁹ VG/ear, 5×10⁹ VG/ear, 6×10⁹ VG/ear, 7×10⁹ VG/ear, 8×10⁹ VG/ear, 9×10⁹ VG/ear, 1×10¹⁰ VG/ear, 2×10¹⁰ VG/ear, 3×10¹⁰ VG/ear, 4×10¹⁰ VG/ear, 5×10¹⁰ VG/ear, 6×10¹⁰ VG/ear, 7×10¹⁰ VG/ear, 8×10¹⁰ VG/ear, 9×10¹⁰ VG/ear, 1×10¹¹ VG/ear, 2×10¹¹ VG/ear, 3×10¹¹ VG/ear, 4×10¹¹ VG/ear, 5×10¹¹ VG/ear, 6×10¹¹ VG/ear, 7×10¹¹ VG/ear, 8×10¹¹ VG/ear, 9×10¹¹ VG/ear, 1×10¹² VG/ear, 2×10¹² VG/ear, 3×10¹² VG/ear, 4×10¹² VG/ear, 5×10¹² VG/ear, 6×10¹² VG/ear, 7×10¹² VG/ear, 8×10¹² VG/ear, 9×10¹² VG/ear, 1×10¹³ VG/ear, 2×10¹³ VG/ear, 3×10¹³ VG/ear, 4×10¹³ VG/ear, 5×10¹³ VG/ear, 6×10¹³ VG/ear, 7×10¹³ VG/ear, 8×10¹³ VG/ear, 9×10¹³ VG/ear, 1×10¹⁴ VG/ear, 2×10¹⁴ VG/ear, 3×10¹⁴ VG/ear, 4×10¹⁴ VG/ear, 5×10¹⁴VG/ear, 6×10¹⁴VG/ear, 7×10¹⁴VG/ear, 8×10¹⁴VG/ear, 9×10¹⁴VG/ear, 1×10¹⁵ VG/ear, or 2×10¹⁵ VG/ear).

In some embodiments, the AAV vectors may be administered to the subject at a dose of about 1×10⁷ VG/retina to about 2×10¹⁵ VG/retina (e.g., 1×10⁷ VG/retina, 2×10⁷ VG/retina, 3×10⁷ VG/retina, 4×10⁷ VG/retina, 5×10⁷ VG/retina, 6×10⁷ VG/retina, 7×10⁷ VG/retina, 8×10⁷ VG/retina, 9×10⁷ VG/retina, 1×10⁸ VG/retina, 2×10⁸ VG/retina, 3×10⁸ VG/retina, 4×10⁸ VG/retina, 5×10⁸ VG/retina, 6×10⁸ VG/retina, 7×10⁸ VG/retina, 8×10⁸ VG/retina, 9×10⁸ VG/retina, 1×10⁹ VG/retina, 2×10⁹ VG/retina, 3×10⁹ VG/retina, 4×10⁹ VG/retina, 5×10⁹ VG/retina, 6×10⁹ VG/retina, 7×10⁹ VG/retina, 8×10⁹ VG/retina, 9×10⁹ VG/retina, 1×10¹⁰ VG/retina, 2×10¹⁰ VG/retina, 3×10¹⁰ VG/retina, 4×10¹⁰ VG/retina, 5×10¹⁰ VG/retina, 6×10¹⁰ VG/retina, 7×10¹⁰ VG/retina, 8×10¹⁰ VG/retina, 9×10¹⁰ VG/retina, 1×10¹¹ VG/retina, 2×10¹¹ VG/retina, 3×10¹¹ VG/retina, 4×10¹¹ VG/retina, 5×10¹¹ VG/retina, 6×10¹¹ VG/retina, 7×10¹¹ VG/retina, 8×10¹¹ VG/retina, 9×10¹¹ VG/retina, 1×10¹² VG/retina, 2×10¹² VG/retina, 3×10¹² VG/retina, 4×10¹² VG/retina, 5×10¹² VG/retina, 6×10¹² VG/retina, 7×10¹² VG/retina, 8×10¹² VG/retina, 9×10¹² VG/retina, 1×10¹³ VG/retina, 2×10¹³ VG/retina, 3×10¹³ VG/retina, 4×10¹³ VG/retina, 5×10¹³ VG/retina, 6×10¹³ VG/retina, 7×10¹³ VG/retina, 8×10¹³ VG/retina, 9×10¹³ VG/retina, 1×10¹⁴ VG/retina, 2×10¹⁴ VG/retina, 3×10¹⁴ VG/retina, 4×10¹⁴ VG/retina, 5×10¹⁴VG/retina, 6×10¹⁴VG/retina, 7×10¹⁴VG/retina, 8×10¹⁴VG/retina, 9×10¹⁴VG/retina, 1×10¹⁵ VG/retina, or 2×10¹⁵ VG/retina).

The compositions described herein are administered in an amount sufficient to improve hearing, vision, or vestibular function, and increase Pcdh15 or a variant thereof expression (e.g., expression of Pcdh15 in a cochlear hair cell, e.g., an inner hair cell), or increase Pcdh15 function. Hearing or vision may be evaluated using standard hearing tests (e.g., audiometry, ABR, electrocochleography (ECOG), and otoacoustic emissions) and may be improved by 5% or more (e.g., 5%, 10%, 15%, 20%, 30%, 40%, 50%, 60%, 70%, 80%, 90%, 100% or more) compared to hearing measurements obtained prior to treatment. In some embodiments, the compositions are administered in an amount sufficient to improve the subject's ability to understand speech.

The compositions described herein may also be administered in an amount sufficient to slow or prevent the development or progression of sensorineural hearing loss (e.g., in subjects who carry a mutation in Pcdh15 or have a family history of autosomal recessive hearing loss but do not exhibit hearing impairment, or in subjects exhibiting mild to moderate hearing loss). Pcdh15 expression may be evaluated using immunohistochemistry, Western blot analysis, quantitative real-time PCR, or other methods known in the art for detection protein or mRNA, and may be increased by 5% or more (e.g., 5%, 10%, 15%, 20%, 30%, 40%, 50%, 60%, 70%, 80%, 90%, 100% or more) compared to Pcdh15 expression prior to administration of the compositions described herein. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months or more following administration of the composition depending on the dose and route of administration used for treatment. Depending on the outcome of the evaluation, the patient may receive additional treatments.

### Kits

The compositions described herein can be provided in a kit for use in treating sensorineural hearing loss, vision loss, or vestibular dysfunction. Compositions may include nucleic acid vectors (e.g., AAV vectors) described herein (e.g., a first nucleic acid vector containing a polynucleotide that encodes and N-terminal portion of a Pcdh15 or variant and a second nucleic acid vector containing a polynucleotide that encodes a C-terminal portion of Pcdh15 or variant thereof), optionally packaged in an AAV virus capsid (e.g., an AAV1 capsid). The kit can further include a package insert that instructs a user of the kit, such as a physician, to perform the methods described herein. The kit may optionally include a syringe or other device for administering the composition.

All publications herein are incorporated by reference to the same extent as if each individual publication or patent application was specifically and individually indicated to be incorporated by reference.

### EXAMPLES

### Example 1. Dual AAV Based PCDH15 Gene Therapy Achieves Sustained Rescue of Visual Function in Mice and Gene Signatures in Patient-Derived Retinal Organoids

Mutations in the PCDH15 gene, encoding protocadherin-15, are amongst the leading causes of Usher syndrome type 1 (USH1F) worldwide. A founder truncating variant of PCDH15 has a ˜2% carrier frequency in Ashkenazi Jews accounting for nearly 60% of their USH1 cases. Although cochlear implants can restore hearing perception in USH1 patients, presently there are no effective treatments for the vision loss due to retinitis pigmentosa. We established patient-derived retinal organoids (ROs), and a founder allele-specific Pcdh15 knockin mouse model as platforms to ascertain therapeutic strategies. Using a dual-vector approach to circumvent the size limitation of adeno-associated virus (AAVs), we observed robust expression of exogenous PCDH15 in the retinae of Pcdh15^{KI} mice, sustained recovery of electroretinogram (ERG) amplitudes and key retinoid oxime, substantially improved light-dependent translocation of phototransduction proteins, and enhanced retinal pigmented epithelium derived enzymes. Transduction of these constructs in patient-derived ROs resulted in improved expression of PCDH15. Thus, in two separate patient-associated models our data raises the hope and paves the way for future gene therapy trials in USH1F subjects.

### Results

### Development of Dual AAV Based PCDH15 Gene Delivery Strategy

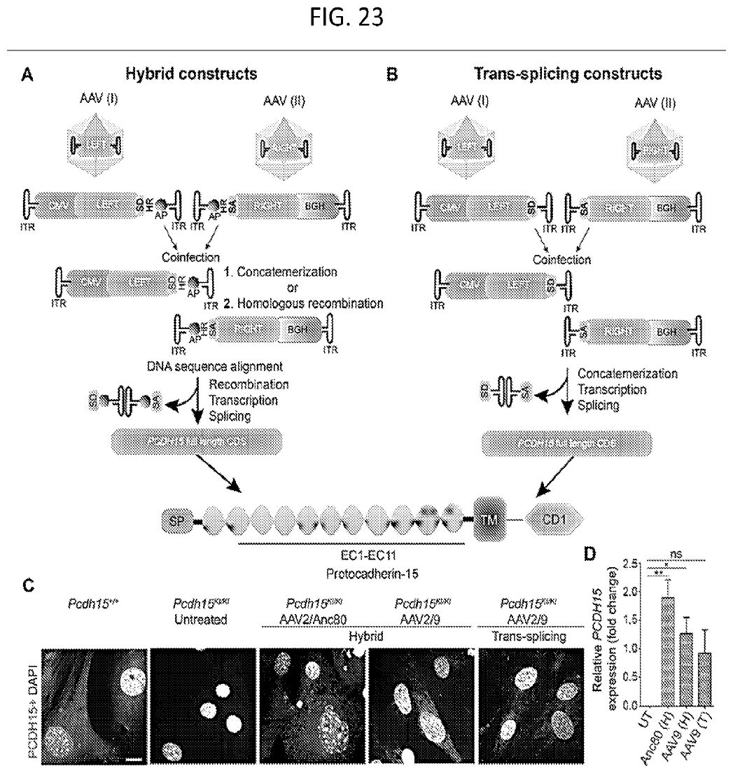

To develop gene-replacement based treatment for vision impairment in USH1F subjects, we used our recently reported Pcdh15^{KI} mice, and designed a AAV-based gene augmentation strategy (Sethna et al., Elife, (2021), 10; Carvalho et al., Front Neurosci, (2017), 11:503). In humans, the coding sequence for PCDH15 is ˜5.7 Kb, too large to fit in a single AAV vector, therefore, to circumvent this limitation, we designed and compared dual-vector approach using hybrid and trans-splicing recombination strategies (Carvalho et al., Front Neurosci, (2017), 11:503). The longest human isoform of the PCDH15 (ENST00000373957.7), encoding a 1962 amino acid polypeptide (NCB1 accession: CCDS73137) was split into two halves at a site that lacks predicted structural domains, and yield similar-sized fragments for packaging into AAV vectors. For the hybrid approach, part of the human alkaline phosphatase placental gene sequence was fused with the PCDH15 constructs (Carvalho et al., Front Neurosci, (2017), 11:503). To evaluate recombination of full-length PCDH15, dual plasmids encoding the hybrid, or trans-splicing strategies, were transfected into USH1F patient derived primary fibroblast cells. Cells lysates were collected and analyzed by RT-PCR using primers flanking the recombination sites. After confirming the expression of full-length PCDH15, we packaged constructs of both hybrid and trans-splicing approaches, into two different AAV serotypes; AAV2/9 and AAV2/Anc80L65 (FIGS. 23A and B). These serotypes were selected based on their high transduction efficiency in the retinal sensory cells, including light-sensing photoreceptors (Carvalho et al., Front Neurosci, (2017), 11:503; Carvalho et al., Hum Gene Ther, (2018), 29:771-784; Vandenberghe et al., PLOS One, (2013), 8: e53463).

### In Vitro Evaluation of Dual AAV PCDH15 Vectors

To evaluate recombination of full-length protocadherin-15, encoded by exogenous PCDH15 gene delivery, we first, infected adipose derived mesenchymal stem cells (MSCs) from Pcdh15^{KI/+} (control) and Pcdh15^{KI/KI} (mutant) mice with our three different dual AAV constructs. Using an antibody directed against the cytoplasmic domain of the protein (Ahmed, Z. M., et al., Hum Mol Genet, (2003), 12:3215-3223; Ahmed et al., J Neurosci, (2006), 26:7022-7034), we found comparable restoration of expression of full length protocadherin-15 with all three dual AAV constructs (FIG. 23C), indicating that both hybrid and trans-splicing approaches were able to recombine and generate full length PCDH15 mRNA.

Next, we infected USH1F patient derived primary dermal fibroblasts with each of these dual AAV constructs and assessed the full-length PCDH15 mRNA levels five days post-infection. Compared to non-transfected cells, we found varying levels of PCDH15 in cells infected simultaneously with both vectors (FIG. 23D). However, the hybrid approach constructs packaged into Anc80/L65 capsid yielded highest expression (FIG. 23D), thus we selected these constructs for in vivo studies in Pcdh15^{KI/KI} mice.

### Subretinal Dual-Vector Delivery Restores Retinal Protocadherin-15 Expression and Photoreceptor Function

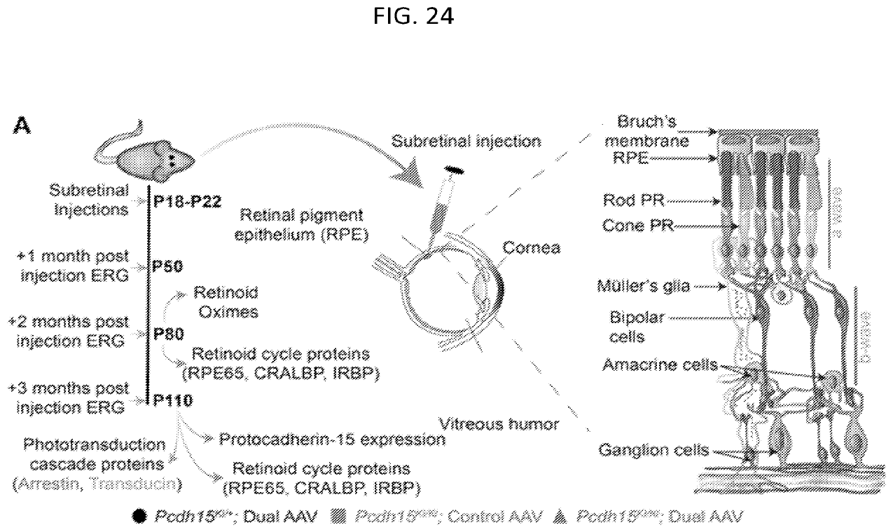

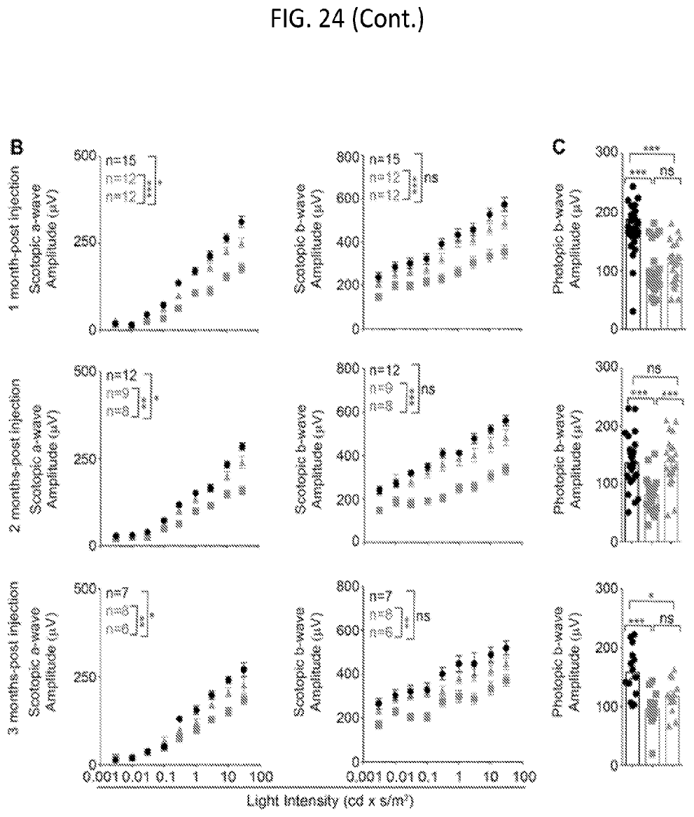

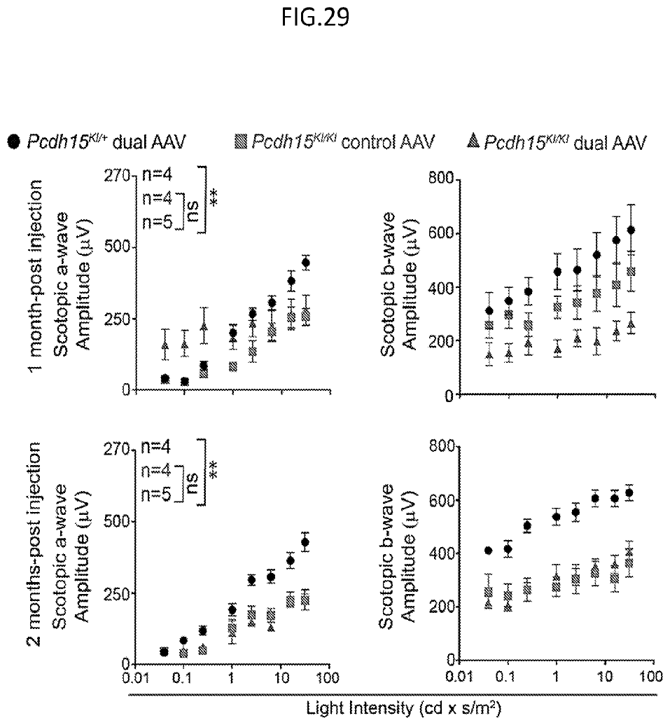

Next, to rescue the retinal phenotype in the Pcdh15^{KI} mice, we delivered our dual hybrid AAV PCDH15 vectors subretinally to maximize photoreceptor targeting (FIG. 24A). Our first cohort of mice were injected in both eyes at P30 with either 1 μl of the AAV2/Anc80L65 PCDH15 left end vector (control) or 1 μl of a 1:1 dilution of both left and right end of our AAV2/Anc80L65 PCDH15 vectors (dual vectors; treatment). Retinal function of these mice was evaluated via ERGs, 1- and 2-months post injections, and compared to Pcdh15^{KI/+} control littermates (FIG. 29). We observed no statistically significant improvement in the ERG amplitudes in Pcdh15^{K/KI} mutant mice that received dual viruses as compared to control AAV (FIG. 29). We reasoned that the failure of rescue of visual function could be due to the more mature development stage of the retina.

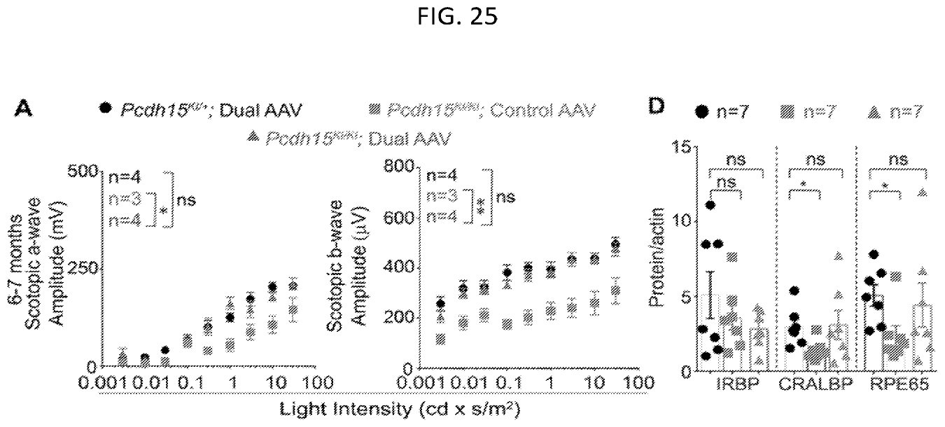

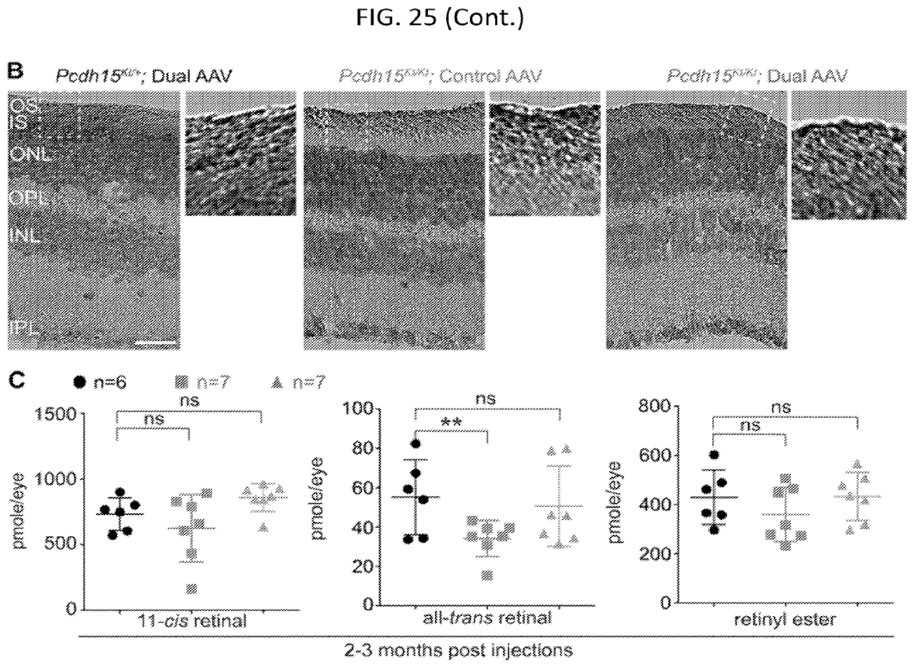

Next, we injected a cohort of control and mutant mice at an earlier age (P18-P22) and evaluated the temporal rescue of retinal functions at specific ages (schematic of treatment paradigm and biomarkers assessment; FIG. 24A). Scotopic ERGs, which preferentially analyze rod photoreceptor function, revealed significant improvement in both, a-wave and b-wave amplitudes in mutant mice treated with dual AAV vectors as early as 1-month post-injection as compared to control injected mutant mice (FIG. 24B). Similarly, the photopic responses, which preferentially measure cone photoreceptor function, were also rescued in dual AAV injected Pcdh15^{KI/KI} mice (FIG. 24C). As expected, mutant mice injected with control left end AAV did not rescue the ERG amplitudes (FIG. 24B). Finally, the recovery of ERG amplitudes persisted out to 28 weeks after injection, the latest time point tested (FIG. 25A). Consistent with ERG findings, twelve weeks after subretinal injections of the hybrid AAV2/Anc80L65 vector pair, we found restoration of protocadherin-15 expression in the photoreceptors of dual AAV treated mutant mice as compared to mutants treated with the control left end vector (FIG. 25B). These data demonstrates that that full length PCDH15 is reconstituted in retinal photoreceptors efficiently with sustained expression at older ages.

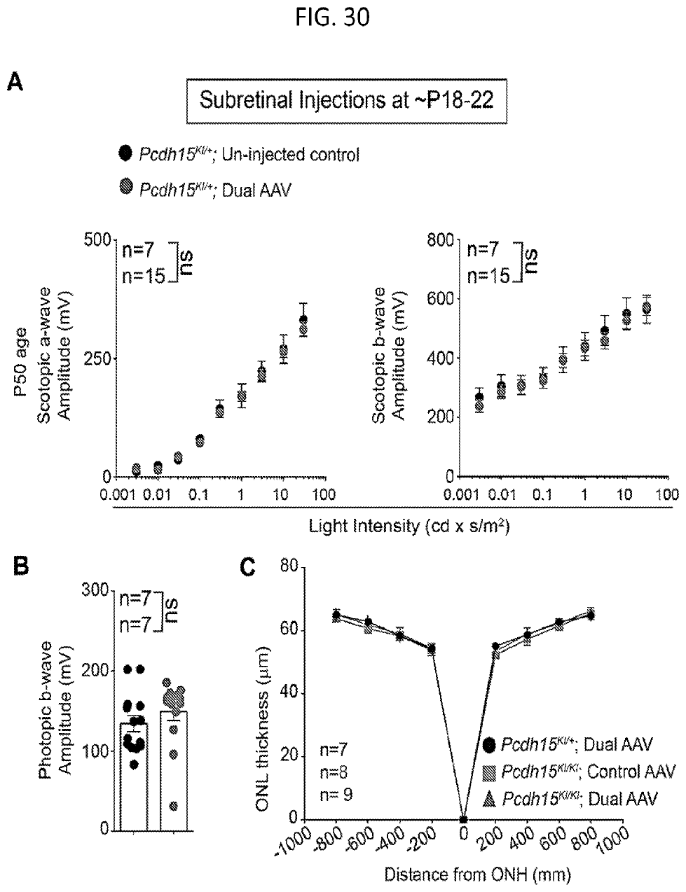

As expected, AAV mediated subretinal delivery of PCDH15 showed no apparent retinal degeneration in treated mutant mice as measured by non-invasive imaging optical coherence tomography (OCT; FIG. 30C). Finally, the Pcdh15^{KI/+} mice that received sub-retinal injection of dual AAV vectors had no changes in ERG amplitudes when compared with uninjected mice (FIG. 30A-B), indicating that exogenous expression of PCDH15 fragments, and full-length protocadherin-15 protein had no overt consequences for normal retinal function. Taken together, these results suggest that dual vector gene replacement of full-length protocadherin-15 is safe for the retina and can restore photoreceptors function in a patient-relevant mouse model of USH1F.

### Dual-Vector Delivery Restores Protocadherin-15 Mediated Functions in the Retina

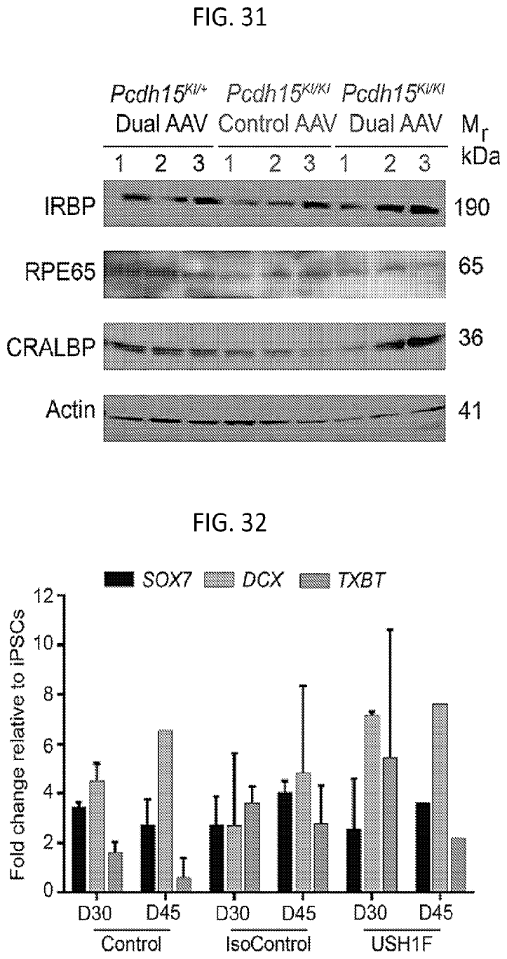

As shown in our previous study, loss of protocadherin-15 resulted in impaired levels of crucial retinoid cycle proteins: RPE65, an essential isomerase which catalyzes the conversion of all-trans retinyl ester to 11-cis retinol, and CRALBP (cellular retinaldehyde-binding protein), a key retinoid transporter (Sethna et al., Elife, (2021), 10). Moreover, Pcdh15^{KI/KI} mice had reduced levels of retinoids (Sethna et al., Elife, (2021), 10). Thus, we next analyzed the impact of PCDH15 gene replacement on the levels of visual cycle proteins and retinoid oxime in the retinae of Pcdh15^{KI/KI} mice. Intriguingly, immunoblotting revealed significantly improved levels of RPE65 and CRALBP at 2-3 months post-treatment, in Pcdh15^{KI/KI} mice that received dual AAV vectors, but not in mutant mice that had received the control right end AAV vector only (FIG. 25D; and FIG. 31). Next, we quantified the retinoids levels one hour after dark adaptation following bleaching with 15,000 lux for one hour (Li et al., Hum Mutat, (2019), 40:426-443). Dual AAV treated mutant mice had retinoid levels that were comparable to Pcdh15^{KI/+} control mice (FIG. 25C).

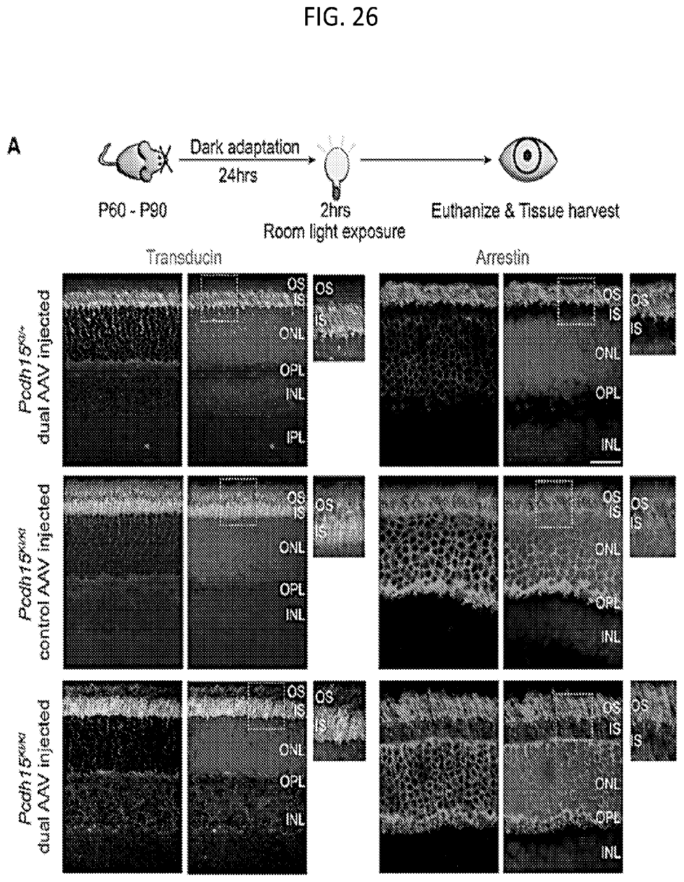

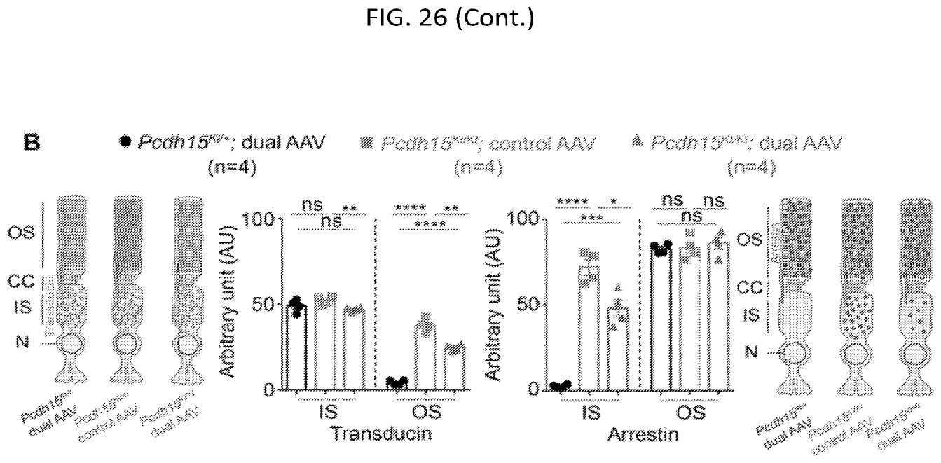

Arrestin and transducin shuttle between inner (IS) and outer segments (OS) of photoreceptors in response to light and dark conditions and are important for phototransduction cascade reaction termination (Burns et al., Annu Rev Neurosci, (2001), 24:779-805; Arshavsky et al., Neuron, (2002), 36:1-3). We previously reported significant mislocalization of both arrestin and transducin to the photoreceptor IS and OS in mutant mice, only under light-adapted conditions (Sethna et al., Elife, (2021), 10). For these studies, ˜3 months post-injected mice after overnight dark adaptation followed by exposure to normal room light for 2 hr and their retinae were examined for localization of arrestin and transducin. We found partial rescue of localization of transducin and arrestin in mutant mice after PCDH15 gene delivery compared to control mice. (FIG. 26A-B). Taken together, these results suggest that exogenous expression of PCDH15 restored key visual cycle proteins expression, retinoid synthesis, and phototransduction proteins shuttling in mutant mice.

### Development and Validation of an USH1F-Patient Derived Retinal Organoids (ROs) Model

Next to further translate the exciting pre-clinical mouse model data to human retinal tissue, we generated hiPSCs from the dermal fibroblasts of an USH1F subject harboring homozygous p.Arg245* variant, and from a healthy control individual. Next, we generated a mutation-corrected isogenic control cell line of USH1F (IsoCon) through CRISPR-based genome editing.

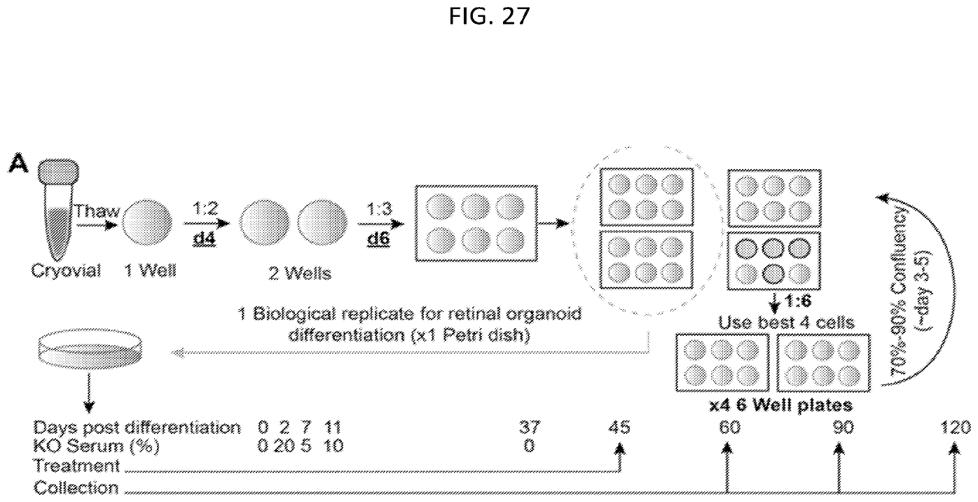

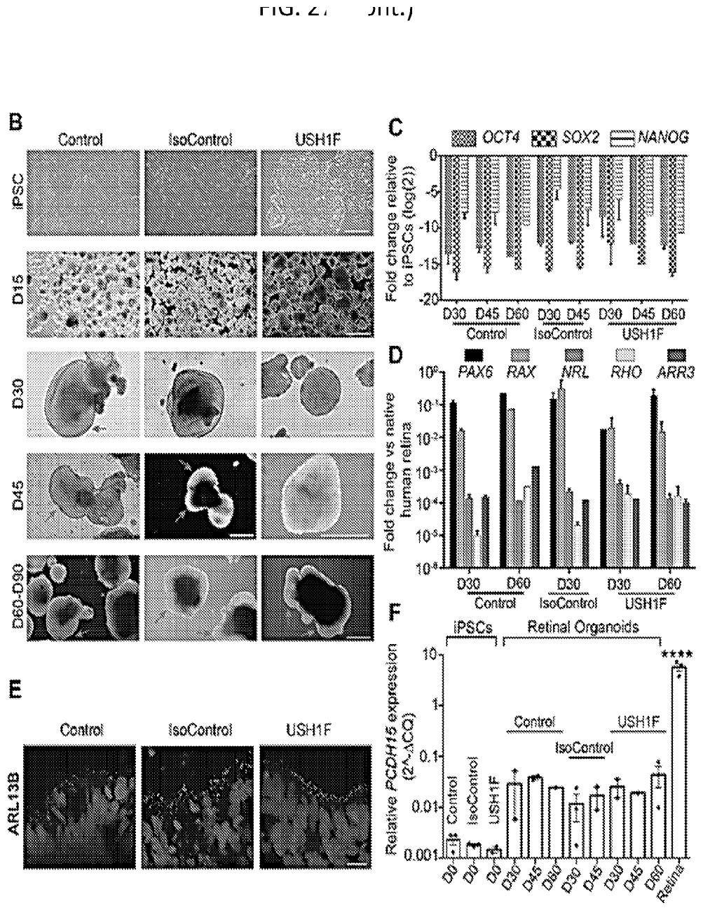

These iPSCs were expended and differentiated into ROs (schematic, FIG. 27A). All three lines (control, IsoControl and USH1F) developed neuroepithelium over time (FIG. 27B). Upon differentiation, pluripotency markers OCT4, SOX2 and NANOG were downregulated (FIG. 27C), while germ layer markers TBXT, SOX7 and DCX were upregulated when compared to the levels of expression in non-differentiating iPSC (FIG. 32). Next, differentiation of iPSCs into ROs was further confirmed through temporal expression profiling for retinal progenitors (PAX6, RAX), photoreceptor cells specific markers (NRL, RHO), and a cone-specific marker (ARR3) (FIG. 27D). Finally, the presence of the developing connecting cilium, labeled with anti-ARL13B antibody, indicated morphological maturation of photoreceptors in iPSCs-derived ROs (FIG. 27E). No noticeable difference among the patient derived versus control ROs were observed for any of the above markers (FIG. 27C-E). Next, we assessed endogenous expression of PCDH15 in patient and wild type ROs relative to native human retina at multiple time points. PCDH15 expression is significantly lower in iPSCs and ROs samples relative to native human retina, however, similar expression was found within the different tested samples (FIG. 27F), indicating that despite the presence of the p.Arg245* mutation, PCDH15 mRNA escapes nonsense mediated decay in USH1F ROs (FIG. 27F).

Dual-AAV Based Gene Delivery Improved the Expression of PCDH15 in iPSC-Derived ROs

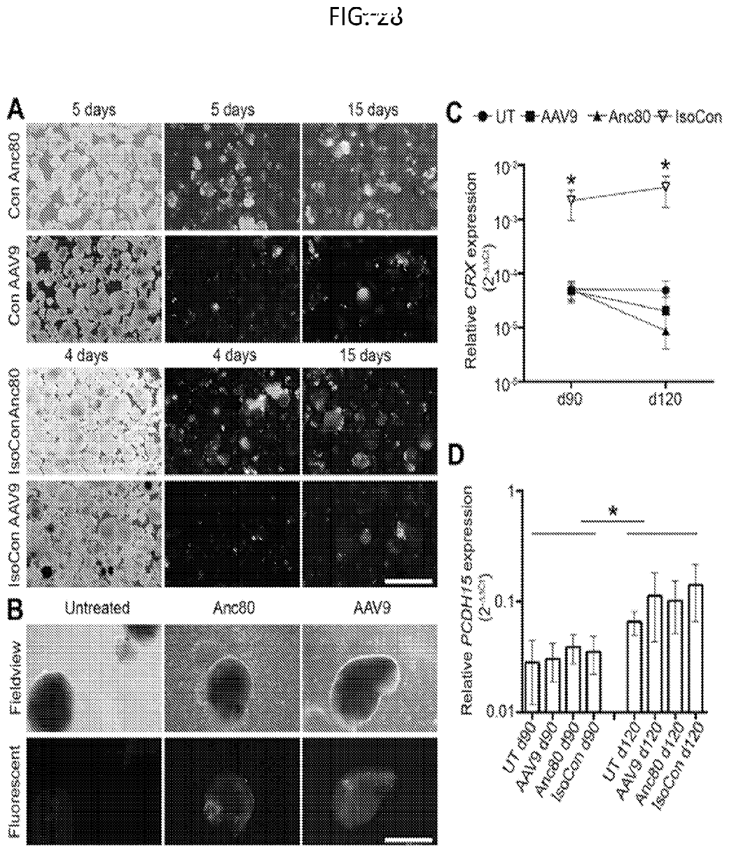

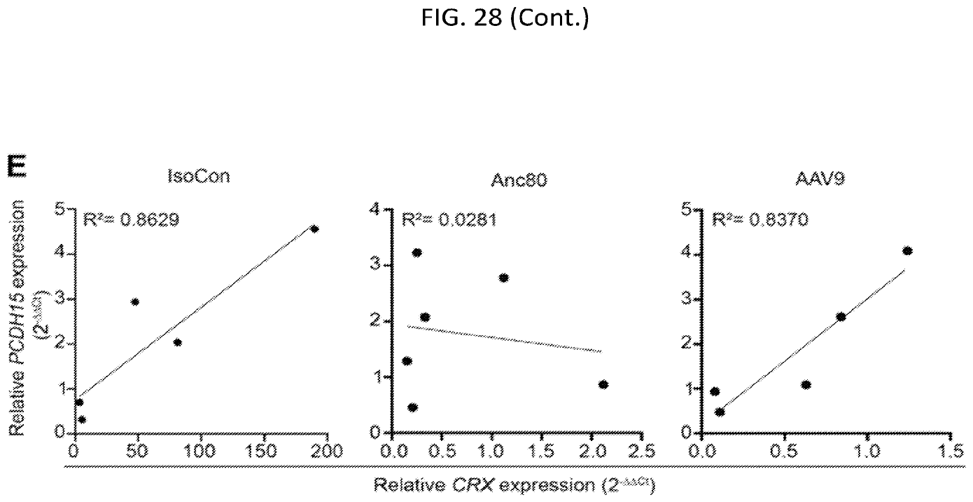

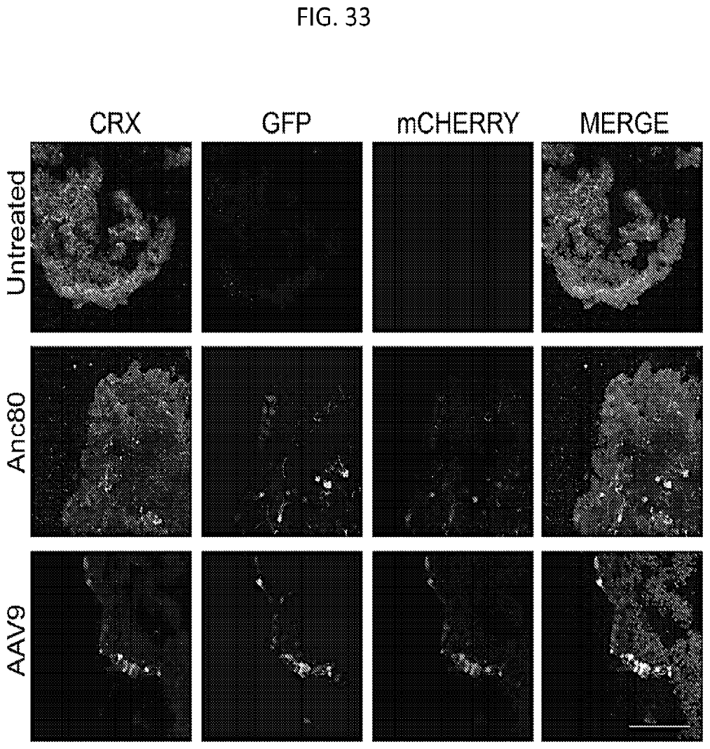

We next assessed the relative serotype efficacy in ROs for single and dual AAV co-transduction. For single AAV transduction efficacy, we found observable GFP expression with both AAV2/Anc80L65 and AAV2/9 serotypes with persistently higher expression of GFP with AAV2/Anc80L65 transduced ROs (FIG. 28A). Next, to determine the co-transduction efficiency, we packaged viral capsids either with GFP or mCHERRY reporter cassettes. ROs were co-transduced at day 45 post-differentiation and imaged 14 days post-transduction, confirming expression of both reporters with both serotypes (FIG. 28B). To confirm the expression of both serotypes in developing photoreceptors, we immunolabeled the ROs with anti-CRX antibodies (an early photoreceptor marker) and found comparable results for either capsid (FIG. 33). To rule out any apparent toxic effects on AAV transduction, we analyzed expression of CRX, a photoreceptor-specific transcription factor crucial for photoreceptor differentiation, at later time points and found no significant changes (FIG. 28C).

Next, we assessed ROs transduced with dual AAV vectors, which revealed improved PCDH15 expression over-time (FIG. 28D). No statistically significant difference between AAV2/9 vs AAV2/Anc80L65 treatment groups indicating both serotypes carrying PCDH15 constructs had similar gene expression levels in ROs (FIG. 28D).

To further probe PCDH15 expression in ROs, we compared PCDH15 expression relative to CRX expression. We found strong correlation of CRX expression with PCDH15 in isogenic control ROs. However, ROs transduced with dual AAV2/Anc80L65 PCDH15 constructs did not correlate to CRX expression over the assessed time course. In contrast, AAV2/9 transduced ROs revealed a positive correlation with CRX expression, indicating that AAV2/9 might have a better transduction efficiency of photoreceptors as compared to AAV2/Anc80L65, despite overall lower transduction (FIG. 28E). Taken together, our study revealed improved expression of PCDH15 in USH1F-ROs transduced with dual AAV capsid constructs.

### Discussion

Here, we demonstrated that efficient gene delivery into the neurosensory retina with the dual-AAV capsids was able to rescue visual function in a patient relevant mouse model of USH1F. We previously reported that mice homozygous for p.Arg250* truncating variant of Pcdh15 have significantly attenuated ERG amplitudes, and deficits in their visual cycle and retinoid synthesis, without overt cell loss (Sethna et al., Elife, (2021), 10). The pcdh15b zebrafish mutants also displayed vision deficits and structural deficits in the photoreceptors and synaptic regions (Miles et al., Dis Model Mech, (2021), 14). Taken together, these observations strongly suggest that protocadherin-15 plays multiple roles in the retina, and is essential for photoreceptors, synaptic, and retinal pigment epithelium functions (Sethna et al., Elife, (2021), 10; Miles et al., Dis Model Mech, (2021), 14). Considering the high prevalence of USH1F in the Ashkenazi Jewish population with a carrier frequency of 2%, and 7-12% worldwide estimate, we tested dual vector gene therapy approaches in a pre-clinical Pcdh15 mutant mouse model to rescue retinal functional deficits (Brownstein et al., Pediatr Res, (2004), 55:995-1000; Ben-Yosef, T., et al., N Engl J Med, (2003), 348:1664-1670; Vozzi et al., Mol Vis, (2011), 17:1662-1668).

We found that recombination of full-length PCDH15 led to restoration of expression of protocadherin-15 in the photoreceptors, recovery of ERG amplitudes, improved retinoid oximes, and RPE enzymes levels. The sustained recovery of ERG amplitudes persisted several months post-injection. We further found that PCDH15 delivery also resulted in significantly improved rescue of phototransduction proteins, arrestin and transducin's light-dependent translocation between inner and outer segments of photoreceptors. We speculate that the partial rescue in the shuttling of visual cycle proteins could be due to limited transduction efficiency of dual-AAV vectors, amount of protocadherin-15 protein being produced, exogenous gene delivery age, or specific spatio-temporal requirement. It is possible that to increase the efficiency of our dual AAV-PCDH15 constructs, alternate approaches including use of intein-mediated protein recombination,⁴⁴ other synthetic capsids with higher transduction efficiency, or repeated dosing of dual-AAV PCDH15 constructs, may be required to fully rescue arrestin-transducin translocation during light cycle (Tornabene et al., Sci Transl Med, (2019), 11; Croze et al., Int Ophthalmol Clin, (2021), 61:59-89; Dalkara et al., Sci Transl Med, (2013), 5:189ra176; Khabou et al., Biotechnol Bioeng, (2016), 113:2712-2724; Ramachandran et al., Hum Gene Ther, (2017), 28:154-167). Taken together, we have demonstrated improvements in several functional endpoints and biomarkers which can be further assessed in larger species, such as minipigs and NHP, prior to trials in humans.

AAV vectors are a clinically favorable modality for retinal gene transfer, and are currently being used in the clinics for gene delivery and treatment of retinal diseases (Lee et al., Transl Vis Sci Technol, (2019), 8:14; Cheng et al., Hum Gene Ther, (2022), 33:865-878; Russell et al., Lancet, (2017), 390:849-860). To overcome the size limitation of AAV vectors, we adopted split gene strategies, trans-splicing and AP-hybrid approaches (Carvalho et al., Front Neurosci, (2017), 11:503). In agreement with previous reports, we found higher expression of full length gene via AP-hybrid approach, as this approach prevents random concatemerization (Carvalho et al., Front Neurosci, (2017), 11:503; Ghosh et al., Hum Gene Ther, (2011), 22:77-83; Duan et al., Mol Ther, (2001), 4:383-391; Duan et al., Methods Mol Biol, (2003), 219:29-51; Ghosh et al., Mol Ther, (2008), 16:124-130; Yan et al., Methods Enzymol, (2002), 346:334-357; Yan et al., Proc Natl Acad Sci USA, (2000), 97:6716-6721). Hence the AP-hybrid approach may also be useful for gene delivery of other USH genes (e.g. MYO7A, CDH23) that are too large to be packaged into single AAV vectors. After validation studies, we selected AAV2/Anc80L65 serotype for gene delivery in Pcdh15K mice for specific reasons including; a) their high transduction efficiency for retinal pigment epithelium and photoreceptors, b) faster onset and sustained expression in murine and non-human primates (NHP) retina, c) minimal adverse side effects (Carvalho et al., Hum Gene Ther, (2018), 29:771-784). We utilized sub-retinal injection as a method of delivery due to its ability to target macula in NPHs and humans and high vector delivery to photoreceptors, retinal pigment epithelium and Müller glial cells compared to intravitreal approach which preferentially target ganglion cell layer and not the outer retina due to the shielding properties of the inner limiting membrane (Buck et al., Int J Mol Sci, (2020), 21). In agreement with preclinical and clinical data for subretinal injections we did not observe gross adverse impact of our subretinal injections to the integrity of the retina (Miraldi et al., Ophthalmic Genet, (2018), 39:671-677; Carvalho et al., Front Neurosci, (2017), 11:503; Carvalho et al., Hum Gene Ther, (2018), 29:771-784; Ramachandran et al., Hum Gene Ther, (2017), 28:154-167; Russell et al., Lancet, (2017), 390:849-860; Lee et al., Mol Ther Methods Clin Dev, (2019), 13:55-66; Zinn et al., Cell Rep, (2015), 12:1056-1068). Finally, with regards to potential toxicity caused by high dosage or by expression of the transgenes in other cell populations, such as bipolar cells or amacrine cells, we observed no overall adverse effect on ERG amplitudes in control mice injected with dual-AAV vector constructs. Neither was there any apparent change in mice behavior and overall health. Similarly, the mutant mice that received the dual-AAV constructs had improved ERG amplitudes persisting throughout 28 weeks post injection, the latest time point tested, further ruling out any obvious adverse impact of AAV2/Anc80L65 capsids. These observations are in agreement with the previous studies on the use of this AAV capsid in the retina (Carvalho et al., Front Neurosci, (2017), 11:503; Hsu et al., Mol Ther Nucleic Acids, (2023), 31:164-181; Wang et al., PLOS One, (2017), 12, e0182473).

Although PCDH15 dysfunction leads to progressive degeneration of retinal photoreceptor cells in humans, it is important to note that Pcdh15 mutant mice on C57BL/6J background and under normal light conditions do not recapitulate these degenerative features (Sethna et al., Elife, (2021), 10). Apparently, young USH1F patients also display visual deficits without any significant degeneration of their photoreceptors (Sethna et al., Elife, (2021), 10). Thus, our results suggest that there may be a broad temporal window for gene augmentation therapy to prevent photoreceptors degeneration and progressive vision loss in USH1F subjects.

As a step forward towards evaluation of our dual-AAV constructs as potential therapy for USH1F subjects, we needed to assess the possibility of these constructs to restore PCDH15 expression in human retinal tissues. Therefore, we generated USH1F-patient derived ROs, three-dimensional laminar structures that include all key retinal cell types and display an ultrastructure and laminar organization of the human native retina, and thus are a representative model to study human retinal disorders and therapeutic intervention (Li et al., Front Cell Neurosci, (2021), 15:638439). USH1F subject-derived ROs demonstrated no differences in the expression of retinal progenitor or photoreceptor genes, nor any apparent degeneration of photoreceptor cells observed, which is consistent with the post-developmental, later onset of RP in USH1F subjects (Ahmed et al., Clin Genet, (2003), 63:431-444; Smith et al., Am J Med Genet, (1994), 50:32-38; Sethna et al., Elife, (2021), 10; El-Amraou et al., C R Biol, (2014), 337:167-177). Further affirming the viability of our model for USH1F, we were able to observe rudimentary photoreceptor connecting cilia in patient ROs. Deficiency in pcdh15, has been implicated in abnormalities at the connecting cilium and outer segment, and loss of calyceal processes in frogs and zebrafish models (Schietroma et al., J Cell Biol, (2017), 216:1849-1864; Miles et al., Dis Model Mech, (2021), 14; Seile et al., Development, (2005), 132:615-623). However, initial outer segment biogenesis is reportedly independent of protocadherin-15 (Schietroma et al., J Cell Biol, (2017), 216:1849-1864; Miles et al., Dis Model Mech, (2021), 14).

Next, we assessed the impact of dual AAV-based gene delivery in ROs and found ROs transduced with dual AAV vectors revealed improved PCDH15 expression over-time. No statistically significant difference between the two serotypes (AAV2/9 vs AAV2/Anc80L65) treatment groups indicated that both serotypes carrying PCDH15 constructs had similar expression of the gene in ROs. We found strong correlation of CRX expression with PCDH15 in isogenic control ROs, and partial rescue of this correlation was observed in AAV2/9 transduced ROs, but not in ROs transduced with the AAV2/Anc80L65 serotype. More widespread dissemination of AAV2/Anc80L65 in contrast to AAV2/9, or differential level of protein synthesis, might be the plausible explanations for this outcome, however, further studies are required to determine the precise mechanism.

In conclusion, an excellent safety profile of AAV2/An80L65 and AAV2/9 has been demonstrated in several animal model studies as well as in many ongoing clinical trials. Thus, both serotypes are good candidates for human clinical trials of USH1F. We report here, in the USH1F mouse model, our proof of concept that retinal delivery of a fragmented PCDH15 cDNA through a dual-AAV vector approach can efficiently and effectively restore expression of full-length protocadherin-15 protein, resulting in a sustained improvement of the visual function and molecular deficits of these mice. Furthermore, our dual-AAV approached restored expression of PCDH15 in iPSCs-derived ROs. To our knowledge, our study is the first dedicated study to examine multiple approaches to restore expression and function of PCDH15 in USH1F patient-specific systems.

### Methods

### Animal Model

All animal studies were conducted in accordance with the ARRIVE guidelines and ARVO Statement for the Use of Animals in Ophthalmic and Vision Research and the National Institutes of Health Guide for the Care and Use of Laboratory Animals, and after the approval by the University of Maryland Baltimore Institutional Animal Care and Use Committee. For gene augmentation studies, we used Pcdh15 p.Arg250* knock-in (equivalent to human Arg245*; Pcdh15^{KI}) mice (Sethna et al., Elife, (2021), 10). Mice were housed in a facility with 12 hr of light on/off cycle and fed on standard mouse diet (after weaning) with water available ad libitum. We used CO2 asphyxiation followed by cervical dislocation to euthanized mice.

### AAV Vectors Generation

For gene augmentation purpose, the longest human isoform of human PCDH15 gene, encoding a 1962 amino acid protein (PCDH15-205, Ensemble Transcript ID: ENST00000373957.7, NCBI accession: CCDS73137), was split into halves (left and right ends), and subcloned into Adeno-associated virus (AAV) backbone plasmids by Genewiz (South Plainfield, NJ). Left end constructs had the ubiquitous cytomegalovirus (CMV) promoter to drive gene expression and the first half of the PCDH15 gene (split at 2196 bp), while the right end construct contained the second half of the PCDH15 gene followed by the BGH sequence (FIG. 23A-B). We developed both trans-splice and hybrid approaches constructs (Carvalho et al., Front Neurosci, (2017), 11:503). The hybrid recombinogenic region was derived from the human alkaline phosphatase placental gene (288 bp-c.1008) (Carvalho et al., Front Neurosci, (2017), 11:503). AAV vectors were synthesized at the Gene Transfer Vector Core, Harvard Medical School, Boston, USA. The constructs were packaged into two different serotypes, AAV2/9 and AAV2/Anc80L65, which reportedly have high transduction rates in photoreceptor cells (Carvalho et al., Hum Gene Ther, (2018), 29:771-784; Vandenberghe et al., PLOS One, (2013), 8: e53463). All viral stocks had a titer between 10¹² and 10¹⁴ vector genomes (vg)/mL.

### In Vitro Validation of AAV Constructs

We performed in vitro validation of AAV2/9 hybrid and trans-splicing and AAV2/Anc80L65 hybrid vectors by transducing adipose-derived mesenchymal stem cells (MSC), and USH1F subjects derived primary fibroblast cells.

MSCs were isolated from Pcdh15^{+/+} and Pcdh15^{KI/KI} mice (n=3-4/genotype) as described previously (Sethna et al., Elife, (2021), 10). Briefly, at the age of 2-3 months mice were euthanized and adipose tissue was dissected from a subcutaneous site and thoroughly washed, to remove blood vessels, hairs, and other type of connective tissues, with several changes of 1×PBS and minced for further processing for MSC isolation, expansion, and culturing. For in vitro validation of hybrid and trans-splice constructs, WT and mutant MSCs were grown in 6-well plates on a coverslip. At 60% to 70% confluency MSCs were infected with 2 μl of both hybrid and trans-splicing vectors pairs at the titer 10¹² vector genomes (vg)/mL, followed by changing culture medium to DMEM supplemented with 2% FBS and 1×Pen-Strep. Cells were harvested after five days of infections and fixed in 4% paraformaldehyde for further processing. Dual AAV mediated expression of full length protocadherin-15 was detected using an antibody raised against the C-terminus (PB303; i.e. only full length protein would be detected). DAPI was used to stain cell nuclei.

Following informed consent, skin fibroblasts were collected from a person with Usher Syndrome who was homozygous for the c.733C>T (p.R245X) variant in PCDH15. Skin fibroblasts were cultured in media consisting of Knock-out Dulbecco's Modified Eagle Medium/F-12 (KO-DMEM/F12, 12660012, Gibco) supplemented with 10% v/v fetal bovine serum (FBS, WS-FBS-AU-015, Fisher Biotec) and 1×Penicillin/Streptomycin (Pen/Strep, 15140122, Gibco). For PCDH15 constructs validation studies, fibroblasts were transduced with 5 μl of either hybrid or trans-splicing vector pairs at 10¹² vg/mL followed by changing culture DMEM supplemented with 2% FBS and 1×Pen-Strep. After AAV transduction fibroblasts were incubated in 5% CO2 at 37° C. for 5 days before harvesting for RNA extraction using TRIzol reagent (Thermo Fisher Scientific). A SMART First-Strand cDNA Synthesis Kit (Clontech) and oligo-dT primers were used to synthesized cDNA from mRNA. Realtime semiquantitative PCR reactions in triplicates were carried using SYBR Green probes and PCDH15- or GAPDH-specific primers (Table 4).

TABLE 4
List of genes and primers used in the manuscript

| Gene | Forward primer sequence | Reverse primer sequence |
|---|---|---|
| ARR3 | CCAGAAAAGGCTCAACATCAACT | ACCAGGACAACACCGTCAAT |
| | (SEQ ID NO: 21) | (SEQ ID NO: 22) |
| DCX | GCAGTCTCCCATCTCTACGC | CGAGTCCGAGTCATCCAAGG |
| | (SEQ ID NO: 23) | (SEQ ID NO: 24) |
| GAPDH | TGCACCACCAACTGCTTAGC | GGCATGGACTGTGGTCATGAG |
| | (SEQ ID NO: 25) | (SEQ ID NO: 26) |
| NANOG | CTCCAACATCCTGAACCTCAGC | CGTCACACCATTGCTATTCTTCG |
| | (SEQ ID NO: 27) | (SEQ ID NO: 28) |
| NRL | CACTGACCACATCCTCTCGG | GAGGGTTCCCGCTTTACCTC |
| | (SEQ ID NO: 29) | (SEQ ID NO: 30) |
| OCT4 | CCTGAAGCAGAAGAGGATCACC | AAAGCGGCAGATGGTCGTTTGG |
| | (SEQ ID NO: 31) | (SEQ ID NO: 32) |
| PAX6 | ACATTTAAACTCTGGGGCAGGT | GCTGGGTTGGTGTGTGAGAG |
| | (SEQ ID NO: 33) | (SEQ ID NO: 34) |
| PCDH15 | TGTTTGATCCTTATCTGCCAAG | TTCCAGCATCAGGGTCTGTT |
| | (SEQ ID NO: 35) | (SEQ ID NO: 36) |
| RAX | GTCTGTCGGTCCTGAGCAAA | ATATTGCGTCATGCCAGGGT |
| | (SEQ ID NO: 37) | (SEQ ID NO: 38) |
| RHO | TTTGGAGGGCTTCTTTGCCA | TTACACACCACCACGTACCG |
| | (SEQ ID NO: 39) | (SEQ ID NO: 40) |
| RPL13A | CCTGGAGAAGAGGAAAGAGA | TTGAGGACCTCTGTGTATTTGTCA |
| | (SEQ ID NO: 41) | A (SEQ ID NO: 42) |
| SOX2 | GCTACAGCATGATGCAGGACCA | TCTGCGAGCTGGTCATGGAGTT |
| | (SEQ ID NO: 43) | (SEQ ID NO: 44) |
| SOX7 | TTCTTGTGCCTTGAGTGGCA | ATAGAGGCGGCACTCGGATA |
| | (SEQ ID NO: 45) | (SEQ ID NO: 46) |
| TXBT | ACGATCCTGGGTGTGCGTAA | CCGATGCCTCAACTCTCCAG |
| | (SEQ ID NO: 47) | (SEQ ID NO: 48) |
| VSX2 | GAAGGCATTCAACGAAGCCC | ATACTCCGCCATGACACTGC |
| | (SEQ ID NO: 49) | (SEQ ID NO: 50) |

### Subretinal Delivery of Dual AAV Constructs

Subretinal injections were performed in mice at either ˜P18-P22 or ˜P30. Prior to subretinal injections mice were anesthetized with ketamine-xylazine (100 and 10 mg/kg, respectively) and pupils were dilated with 1% Tropicamide. To perform the subretinal injections, a NaNOFIL syringe with 35GA blunt needle (World Precision Instrument, #NF35BL), filled with 1 μl of PCDH15 AAV virus was used. Using a dissecting microscope, the mouse eye was proposed gently using forceps. The temporal conjunctiva was gently pinched with tipped forceps (COLIBI Suturing 7.5C, #5550060 FT, WPI company), and a small incision was made using a 23G sterile syringe to expose the scleral tissue. The NaNOFIL needle was then inserted parallel to the retina to inject the desired volume of 1 μl. The plunger was depressed slowly over 30 seconds with even pressure. Eyes were cleaned with sterile eyewash, and methylcellulose was applied on both eyes to prevent dehydration and to minimize anesthesia induced cataracts.

### Electroretinography (ERG) and Optical Coherence Tomography (OCT)

ERGs were recorded as previously described (Sethna et al., Nat Commun, (2021), 12:3906). Briefly, following overnight dark-adaptation, mice were anesthetized with ketamine-xylazine (100 and 10 mg/kg, respectively) and pupils were dilated with 1% Tropicamide. ERG waveforms were elicited using consecutively brighter stimuli (0.003962233-3.147314 cd·s/m2) with 5-60 s intervals using the Diagnosys Color Dome Ganzfeld system (Diagnosys Systems, Lowell, MA). Three to five waveforms per intensity were averaged. For photopic response measurement mice were exposed to 30 cds/m² white background light for 3 minutes. Photopic, cone-only, responses were elicited at a single bright flash (3.15 cd·s/m²) under a steady rod-suppressing field of 30 cd·s/m², with 10 waves averaged. Waves were analyzed using the Espion software (Diagnosys Systems, Lowell, MA).

OCT was performed using the Spectralis OCT (Heidelberg Engineering, Heidelberg, Germany). Prior to OCT analysis mice were anesthetized and eyes were dilated with 1% Tropicamide. We used custom-designed plano-concave contact lens micro-M 2.00/5.00 (Cantor & Nissel Ltd, Northamptonshire, UK) to obtain cross sections of the entire retina. Outer nuclear quantification (ONL) was performed as detailed previously (Zeng et al., Invest Ophthalmol Vis Sci, (2016), 57: OCT277-287).

### Immunoblotting

Mouse eyecups were homogenized in CHAPS buffer containing 1× protease inhibitors, 1 mM Na orthovanadate, 10 mM Na glycerophosphate, and 10 mM NaF. Equivalent protein concentrations were fractionated on a 4-20% Bis-Tris gel (Invitrogen) and transferred to polyvinylidene fluoride (PVDF) membrane (Millipore). Membranes were blocked and then probed with the rabbit anti-RPE65 (1:100), CRALBP (1:1000; Santa Cruz Biotechnology Sc-28193), IRBP (1:500; Santa Cruz Biotechnology Sc-25787), and Actin (1:500; clone 13E5 Cell Signaling Technology 4970S) overnight at 4° C., followed by three washes of 30 minutes each in 1×TBST. After washing, blots were incubated with horseradish peroxidase-conjugated anti-rabbit antibody (1:1000, Sigma NA934V, Lot #17640116) for 2 hrs at room temperature, followed by detection using the ECL Prime Western Blotting System (Thermo Fisher Scientific 32,106). Samples were analyzed in triplicate.

### Retinoid Extraction and Analysis

All procedures for retinoid extraction were performed under red safelights as described.²⁸ Overnight dark-adapted mice were euthanized with CO2, eyes enucleated, lens and vitreous removed, followed by freezing the eyecups in pairs, on dry ice, and stored at −80° C. Mouse eyes were homogenized in fresh hydroxylamine buffer (1 ml of 0.1 M MOPS, 0.1 MNH2OH, and pH 6.5). 1 ml ethanol was added, samples were mixed and incubated (30 min in the dark at RT). Retinoids were extracted using previously described method (Sethna et al., Elife, (2021), 10). Retinaloxime standards were prepared from 11-cis retinal (National Eye Institute, NIH), and all-trans retinal and synthetic retinyl palmitate (Sigma-Aldrich, Saint Louis, MO) (Garwin et al., Methods Enzymol, (2000), 316:313-324). Synthetic retinyl palmitate was obtained from Sigma-Aldrich. Retinoids were separated on LiChrospher Si-60 (5 μm) normal phase columns using a mobile phase consisting of 11.2% ethyle acetate, 2% dioxane, and 1.4% octanol (v/v/v) in hexane (HEDO) at a flow rate of 0.6 ml/min. Spectral data were acquired over the range of 250-400 nm. Absorbance was monitored at 350 nm for retinaloximes and at 325 nm for retinyl esters. Peak areas were integrated and quantified using external calibration curves. Data were analyzed using Empower three software (Waters Corp., Milford, MA).

### Immunohistochemistry and Confocal Microscopic Imaging

Mice (2-3-month post injection) were dark adapted overnight and exposed to normal room light for 2 hr after light onset and euthanized after room light exposure and eyes were enucleated following CO2 asphyxiation followed by cervical dislocation. Eyes were immediately fixed in Prefer fixative (Anatech LTD, Battle Creek, MI), paraffin embedded and stained using standard published protocols (Sethna et al., Methods Mol Biol, (2013), 935:245-254; Sethna et al., J Biol Chem, (2016), 291:6494-6506). Briefly, 7 μm sections were deparaffinized, rehydrated in 1×PBS, blocked and permeabilized with 10% normal goat serum/0.3% Triton-X 100 for 2 hr at room temperature (RT), and incubated overnight at 4° C. with the indicated primary antibodies to transducin (1:100 dilution, #Sc-517057, Santa Cruz Biotechnology, Dallas, TX) and arrestin (clone C10C10, 1:25 dilution, kind gift from Drs. Paul Hargrave and Clay Smith, University of Florida, FA). The following day, sections were incubated with Alexa fluor labeled goat secondary antibodies (1:250) and DAPI (Thermo Fisher Scientific, Waltham, MA) to label nuclei. Sections were scanned using the UMSOM core facility Nikon W1 spinning disk microscope and images were processed using FIJI software (Schindelin et al., Nat Methods (2012), 9:676-682). To stain for protocadherin-15, dissected eyes were fixed in mix of 2% glutaraldehyde and 2% paraformaldehyde (Electron Microscopy Sciences, Hatfield, PA) and processed for immunostaining using DAB method, and custom antibody targeting the C-terminus of protocadherin-15 (PB303; 1:200) (Duncan et al., J Biol Methods, (2016), 3; Ahmed, Z. M., et al., Hum Mol Genet, (2003), 12:3215-3223).

### Patient Recruitment

All participants gave informed written consent (McCaughey et al., Cell Stem Cell, (2016), 18:307-308). All experimental work was approved by the Human Research Ethics committees of the Royal Victorian Eye and Ear Hospital (11/1031H, 13/1151H-004), as per the requirements of the National Health & Medical Research Council of Australia (NHMRC) and in accordance with the Declarations of Helsinki.

iPSC Generation, Culture, and Differentiation into Human ROs and AAV Transduction

Two iPSC lines were derived from an Usher 1F patient who previously donated dermal fibroblasts. These fibroblasts were reprogrammed using a previously described protocol (Daniszewski et al., iScience, (2018), 7:30-39). The following human iPSC lines were cultured for differentiation: i) USH1F: iPSCs derived from an Usher 1F patient with homozygous Arg245* mutations (c.733C>T) in exon 8 of PCDH15; ii) IsoCon: mutation-corrected isogenic control of USH1F. iPSCs were maintained and expanded on low-growth factor Matrigel-coated (354230, In Vitro Technologies) 6-well plates, using mTeSR-1 media (85850, STEMCELL Technologies) or Stem Flex, a more robust and growth factor-stable equivalent (A3349401, Gibco), with 1× Pen/strep. All cells were maintained by humidified incubation with 5% CO2 at 37° C. Cultures were passaged every 5 days, or earlier if they had already reached ˜90% confluency. iPSCs were differentiated into embryoid bodies at a 70-90% confluency using a mechanical dissociation technique described previously (Mellough et al., Stem Cells Transl Med, (2019), 8:694-706). Embryoid bodies/retinal organoids were maintained in a humified incubator at 37° C. and 5% CO2. Daily media changes occurred until day 37 post-differentiation, after which media was changed every second day. iPS media mTESR™1 (85850, StemCell Technologies) with 1% ROCK inhibitor Y-27632 (SCM075, Sigma-Aldrich), was used for the first two days post-differentiation. From day 3 onwards, retinal organoid media was used, the concentration of some components gradually changed over time (Table 5).

TABLE 5
Retinal Organoid Media

| | 20% KOSR | 15% KOSR | 10% KOSR | 0% KOSR |
|---|---|---|---|---|
| | Days post-differentiation | | | |
| | 3-8 | 9-12 | 13-37 | 38-120 |
| Knockout Serum | 20% | 15% | 10% | N/A |
| Replacement | | | | |
| (Gibco, USA) | | | | |
| MEM Non- | 1% | 1% | 1% | 1% |
| Essential Amino | | | | |
| Acids (Gibco, | | | | |
| USA) | | | | |
| Penicillin- | 1% | 1% | 1% | 1% |
| Streptomycin | | | | |
| 10,000 U/mL | | | | |
| (Gibco, USA) | | | | |
| GlutaMAX | 1% | 1% | 1% | 1% |
| Supplement | | | | |
| (Gibco, USA) | | | | |
| B27 supplement | 2% | 2% | 2% | 2% |
| (Harry Perkins, | | | | |
| AU) | | | | |
| Recombinant | 5 ng/mL | 5 ng/mL | 5 ng/mL | 10 ng/mL |
| Human IGF-1 | | | | |
| (PeproTech, | | | | |
| USA) | | | | |
| Knockout | 75% | 80% | 85% | 95% |
| DMEM/F-12 | | | | |
| (Gibco, USA) | | | | |

KOSR=Knockout Serum Replacement. Components are given by volume as a percentage of total media.

ROs collected on day 30, 45, and 60 of differentiation underwent transcript analysis by RT-qPCR. All samples were washed with PBS prior to the addition of TriReagent. RNA was extracted via chloroform-TRIzol™ extraction (Buljan, M., et al., Curr Opin Struct Biol, (2013), 23:443-450). The aqueous phase was further purified with a RNeasy® Mini Kit (74104, Qiagen, Germany). Eluted RNA was measured via a Nanodrop 2000 spectrophotometer (Thermo Fisher Scientific) and up to 500 ng was used to synthesize cDNA (Superscript IV first-strand synthesis system, Invitrogen) using 50 μM Oligo d (T) 20 primers. Post-mortem human retina collected from Lions Eye Bank (Nedlands, WA 6009) was used as a positive control and RNA and cDNA extracted as described above. Information on the donor sample is as follows: Donor ID: D21-0680, age post-mortem: 107 hrs; age of donor: 24; sex: female.

RT-qPCR reactions were carried out on 384 well plates (10 μL reaction volume). To assess the development of ROs, SYBR-green master mix (1725151, Bio-Rad) was used in combination with primer sets designed to bind to sequences in pluripotent-, retinal- and housekeeping genes. Reconstitution of the PCDH15 gene was assessed using a TaqMan (TaqMan fast advanced master mix, 4444557, Applied Biosystems) fluorophore-labelled primer/probe assay (Roche Life Sciences) designed to span the gene split region. Samples were loaded in technical triplicates (5 ng/well) and normalized to endogenous housekeeping gene GAPDH. Plates were sealed and centrifuged at 4° C. at 1200 g for 1 minute, then analyzed using a Bio-Rad-CFX 384 touch RT-qPCR machine.

For histology, ROs were collected, washed in 1×PBS and fixed in 4% paraformaldehyde (1004968350, Merck) at room temperature for 1 hour. Paraformaldehyde was then removed and ROs washed three times in PBS before cryoprotecting overnight in PBS with 30% w/v sucrose at 4° C. Cryoprotected ROs were then embedded in Optimal Cutting Temperature (OCT) compound (4586, Scigen), frozen at −20° C. and cut into 10 μm-thick cryosections on a CM3050S cryostat (Leica). For IHC, cryosections were washed in 1×PBS before being incubated with blocking buffer (5% v/v normal goat serum and 0.3% v/v Triton X-100 in PBS) at room temperature for 1 hour. Blocking buffer was removed prior to overnight incubation with primary antibody in antibody diluent (1% w/v bovine serum albumin and 0.3% v/v Triton X-100 in PBS) in a humidified chamber at 4° C. Slides were then washed in antibody diluent and incubated at room temperature with secondary antibody. Subsequently, slides were washed in PBS, cover slipped with Vectashield mounting medium with DAPI (H-1200, Vector Laboratories). Immunostained RO sections were imaged using a confocal microscope (Nikon A1RMP). Images are presented as Z-stack maximum intensity projections taken at 1 μm intervals.

For dual AAV capsid efficacy evaluation at least 10 ROs were transferred in each well of a 6-well plate and maintained in 3 mL/well differentiation media. Purified vectors AAV2/9-GFP or AAV2/Anc80-CMV.GFP for single capsid transduction and AAV2/9-CMV.GFP or AAV2/9-CMV.mCherry and AAV2/Anc80-CMV.GFP or AAV2/Anc80L65-CMV.mCherry were diluted in 1× phosphate-buffered saline (PBS, 10010023, Gibco) and added directly to the media at 1.6×10⁸ vg/organoid on day 30 post differentiation. Every 48 hours half of the medium was replaced with fresh. ROs were collected for analysis at day 4, 5, and 15 post-transduction for single capsid transduction and 14 days post transduction for dual AAV capsid transduction. After AAV capsid evaluation ROs were treated with dual AAV PCDH15 hybrid vectors (AAV2/9 and AAV2/Anc80L65) with 1×10¹⁰ vg/vector/organoid at day 45 post differentiation. ROs were collected at day 90 and day 120 for PCDH15 relative expression analysis by RT-qPCR and flow cytometry. RO dissociation was achieved using Embryoid Dissociation Kit, human and mouse (130-096-348, Miltenyi Biotec, Germany) following manufacturer's instructions.

### Statistical Data Analysis

One-way ANOVA with Tukey's post hoc test or Student's t-test was used to compare the control sample to test samples with the data presented as mean±SEM. Differences with p<0.05 were considered significant. Unpaired t-tests were conducted comparing endogenous PCDH15 expression between cell lines where n>1. Data is presented as mean (where n=1) or mean±SEM, (where n>1). Data were analyzed using GraphPad Prism (GraphPad Software, Inc, La Jolla, CA).

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

1. A dual vector system for expressing a Protocadherin-15 protein or variant thereof in a subject, wherein the Protocadherin-15 protein comprises SEQ ID NO:2 or SEQ ID NO:4, wherein the dual vector system comprises
i) a first vector comprising a first coding polynucleotide that encodes an N-terminal portion of the Protocadherin-15 protein or variant thereof; and
ii) a second vector comprising a second coding polynucleotide that encodes a C-terminal portion of the Protocadherin-15 protein or variant thereof,
wherein the first coding polynucleotide and the second coding polynucleotide that encode the Protocadherin-15 protein or variant thereof do not overlap.

2. (canceled)

3. The dual vector system of claim 1, wherein the variant comprises an amino acid sequence that is at least 90% identical to SEQ ID NO:2 or 4.

4. The dual vector system of claim 1, wherein the first and second vector comprises an adeno-associated virus (AAV).

5. The dual vector system of claim 1, wherein the first and/or second vector comprises an adeno-associated virus (AAV) selected from AAV2/2, AAV2/5, AAV2/9, AAV2/Anc80, AAV-7m8 and R100.

6. The dual vector system of claim 1, wherein the first and second vectors comprise a promoter operably linked to the first and second coding polynucleotide.

7. (canceled)

8. (canceled)

9. (canceled)

10. (canceled)

11. (canceled)

12. (canceled)

13. The dual vector system of claim 1, wherein the first coding polynucleotide encodes amino acids 1-732 of SEQ ID NOS: 2 or 4.

14. The dual vector system of claim 1, wherein the second coding polynucleotide encodes amino acids 733-1962 of SEQ ID NO: 2 or amino acids 733-1790 of SEQ ID NO:4.

15. (canceled)

16. The dual vector system of claim 1, wherein the first vector comprises a first inverted terminal repeat (ITR) sequence at a position that is located 5′ of the first coding polynucleotide, and a second ITR sequence that is that is located 3′ of the first coding polynucleotide, and the second vector comprises a first inverted terminal repeat (ITR) sequence at a position that is located 5′ of the second coding polynucleotide, and a second ITR sequence that is that is located 3′ of the second coding polynucleotide, wherein the promoter is located at a position that is between the first ITR sequence and the first coding polynucleotide and the second ITR is located at a position that is 3′ to the splice donor site on the first vector, and the first ITR is located at a position that is 5′ to the splice acceptor site and the second ITR is located at a position that is 3′ of a poly(A) sequence.

17. (canceled)

18. The dual vector system of claim 16, wherein the first ITRs in the first vector and second vector have at least 85% sequence identity to SEQ ID NO:5, and the second ITRs in the first vector and second vector have at least 80% sequence identity to SEQ ID NO:6.

19. (canceled)

20. (canceled)

21. (canceled)

22. The dual vector system of claim 1, wherein the first vector comprises a polynucleotide sequence at least 60% identical to SEQ ID NO:13 and wherein the second vector comprises a polynucleotide sequence at least 60% identical to SEQ ID NO:14.

23. (canceled)

24. The dual vector system of claim 1, wherein the first vector comprises a polynucleotide sequence at least 60% identical to SEQ ID NO:15 and wherein the second vector comprises a polynucleotide sequence at least 60% identical to SEQ ID NO:16.

25. (canceled)

26. The dual vector system of claim 1, wherein the first vector comprises a polynucleotide sequence at least 60% identical to SEQ ID NO:17 and wherein the second vector comprises a polynucleotide sequence at least 60% identical to SEQ ID NO:18.

27. (canceled)

28. The dual vector system of claim 1, wherein the first vector comprises a polynucleotide sequence at least 60% identical to SEQ ID NO:19 and wherein the second vector comprises a polynucleotide sequence at least 60% identical to SEQ ID NO:20.

29. (canceled)

30. A method of treating sensorineural hearing loss in a subject, comprising administering to the subject one or more compositions comprising a therapeutically effective amount of the dual vector system of claim 1.

31. (canceled)

32. (canceled)

33. The method of claim 30, wherein the subject has been diagnosed as having Usher syndrome type I.

34. The method of claim 30, wherein the dual vector system is injected into the inner ear of the subject.

35. (canceled)

36. (canceled)

37. (canceled)

38. (canceled)

39. (canceled)

40. (canceled)

41. (canceled)

42. (canceled)

43. (canceled)

44. A pharmaceutical composition comprising the dual vector system of claim 1.

45. (canceled)


SEQUENCE LISTING (50 sequences)

SEQ ID NO: 1 - DNA, 5889 bp, Homo sapiens
  source 1..5889: mol_type = other DNA
atgtttcgacagttttatctctggacatgtttagcttcagggatcatcctgggctctctctttgaaatctgcttgggccagtatgatgatgactggcaatatgaggattgcaaactagctaggggaggaccaccagctaccatagttgctattgatgaagaaagtcggaatggtacaattctggtggacaacatgctgatcaaagggactgctggaggaccagaccccaccatagaactttctttaaaggataatgtggattactgggtgttgatggatcctgttaagcaaatgcttttcctgaacagcaccggaagagttctggatagagatccaccgatgaacatacactccattgtggtgcaggtccagtgcatcaacaaaaaagtgggcactattatctaccatgaagtgcgaatagtggtgagagacaggaatgacaactcacccactttcaagcatgaaagctactatgccacagtgaatgagctcactccagttggtaccacaatattcacaggattttcaggagacaatggagctacagatatagatgatggaccaaatggacagatagagtatgttattcagtataatccagatgatccgacatccaatgacacctttgaaattcccctaatgttgactggaaatatagtgttaaggaagaggctcaactatgaagataagactcgctactttgtcataatccaagctaatgaccgtgcccaaaatctgaatgagaggcgaaccaccaccaccactctcacagtggatgttctggatggagatgacttgggtccaatgtttcttccttgtgtccttgtgccaaacactcgtgattgccgtccactcacttatcaagctgccatacctgagttgagaactccggaagaactgaaccccattattgttacgccaccaatccaagccattgatcaggaccggaatattcaaccgccatcagataggccaggaatcctctattccatccttgttgggactcctgaggattacccacgatttttccatatgcatcctaggacagcagaacttagtctcctggagccagtaaacagagactttcaccagaaatttgatttggttattaaggctgaacaagacaatggtcatcctcttcctgcctttgccggtctacacattgaaatactggatgaaaacaatcaaagtccatattttacaatgcccagttatcaaggctatatcctggaatctgccccagtgggagcaaccatttcggacagtctcaatttgacttcacctttaagaatagtagctctggacaaggacatagaagatacaaaagacccagagcttcacctttttctgaatgactacacctcagtcttcaccgtcacacagactggtattactcgctacctcaccttacttcaaccagtggacagggaagaacagcaaacttacaccttttcgataacagcatttgatggtgtacaagaaagtgagccagtcatcgtcaatattcaagtgatggatgcaaatgataacacgccaaccttccctgaaatatcctatgatgtgtatgtttatacagacatgagacctggggacagtgtcatacagctcactgcagtcgacgcagacgaagggtcaaatggggagatcacatatgaaatccttgttggggctcagggagacttcatcatcaataaaacaacagggcttatcaccatcgctccaggggtggaaatgatagtcgggcggacttacgcactcacggtccaagcagcggataatgctcctcctgcagagcgaaggaactccatctgcactgtgtatattgaagtgcttccaccaaataatcaaagccctcctcgcttcccacagctgatgtatagccttgaaattagtgaagccatgagggttggtgctgttttattaaatctacaggcaactgatcgagagggagactcaataacatatgccattgagaatggagatcctcagagagtttttaatctttcagaaaccacggggattctaaccttagggaaagcactggacagggaaagcactgatcgctacattctgatcatcacagcttcagatggcaggccagatgggacctcaactgccacagtaaacatagtggtgacagatgtcaatgacaatgctccagtgtttgatccttatctgccaagaaatttatctgtggtggaagaagaagccaatgcctttgtgggtcaagtaaaagcaacagaccctgatgctggaataaatggtcaagtgcactacagtttgggtaactttaataatctttttcgtatcacatccaatgggagcatttacacagcagtgaagcttaacagagaagtcagggactactatgaacttgttgttgtggcaacagatggagcagtacaccctcgtcattcaactctaaccttggccatcaaggttttggacattgatgataacagtcctgtgttcaccaattcaacatacactgtccttgttgaagagaatttgccagctgggactaccatccttcaaatagaggccaaagatgtcgaccttggagcaaatgtgtcttaccggataagaagcccagaagtgaagcacttttttgcactacatccatttacaggagaactatcgcttttaaggagtttagattatgaggcatttccagaccaagaagcaagtatcacttttctggtagaggcctttgatatttatggaacaatgccacctggtattgctactgtcacagtgattgtaaaggatatgaatgattatcctcctgtctttagtaaacgaatatacaaagggatggtggctccggatgcagtcaagggtacacctatcacaacagtttatgctgaagatgcagaccctcctggattacctgcaagtcgtgtgaggtatagagtagatgatgtacagtttccttaccctgccagtatttttgaagtggaagaagattctggaagagtaataacacgagtcaatcttaatgaagaacctacaacaatttttaagttggtggtggttgcttttgatgatggggagcctgtgatgtccagcagtgccacagtgaagattcttgtcttacatcctggtgagatcccacgcttcacacaggaggaatatagacctcctccagtaagtgaacttgccaccaaagggaccatggttggtgtaatttctgctgctgccattaatcaaagtattgtgtactccattgtttcaggaaatgaagaagatacatttggaattaataacatcacaggtgttatctatgtgaatggacctctggattatgagaccaggacaagctatgtacttcgagtccaagctgattccctggaagtggtccttgccaatctccgagttccttcaaaaagcaatacagctaaagtatacattgagattcaggatgaaaataatcatcccccagtgtttcagaaaaaattctacatcggaggtgtatctgaagatgcaagaatgtttacttctgtactcagagtgaaggctactgataaagatactggcaattatagtgtcatggcctacagactcataataccaccaattaaagagggaaaagaaggatttgtagtggaaacatatacagggcttatcaaaactgctatgctcttccataatatgaggagatcctacttcaagtttcaagttattgcaactgacgactatgggaagggactgagcggcaaagccgatgtactcgtctccgtggtcaatcagctggatatgcaagtcattgtttccaatgtgcctcctactctagtggaaaaaaagatagaagatcttacagagatcttggatcgctatgttcaggaacaaattcctggtgccaaggtcgtagtggagtccattggagctcgccggcatggagatgccttttccctagaagattacaccaaatgtgacttgactgtctatgcaattgacccccaaaccaacagagccatcgatagaaatgagctttttaaatttttggatggcaaactacttgatatcaataaagactttcagccgtattatggggaaggaggacgcattctggagatccggactccagaggcagtgaccagcattaaaaagagaggagaaagtctaggatacacagaaggggccttgttggctctggccttcatcatcatcctctgctgcattcctgccatcttggtggttttggtcagctacagacagtttaaagtacgtcaagctgagtgtacaaagactgcacgaattcaggccgcattacccgcggctaaaccagcagtgccggctcctgcaccagtggcagcgcccccgccgccgccgccgcctccgccaggtgcgcatctctatgaagaacttggagacagctcaatgcataatcttttccttctctaccattttcaacaaagcaggggaaataactcagtctcagaagacaggaaacatcaacaagttgtgatgcccttttcttccaatactattgaggctcacaagtcagctcatgtagacggatcacttaagagcaacaaactgaagtctgcaagaaaattcacatttctatctgatgaggatgacttaagtgcccataatcccctttataaggaaaacataagtcaagtatcaacaaattcagacatttcacagagaacagattttgtagacccattttcacccaaaatacaagccaagagtaagtctctgaggggcccaagagaaaagattcagaggctgtggagtcagtcagtcagcttacccaggaggctgatgaggaaagttccaaatagaccagagatcatagatctgcagcagtggcaaggcaccaggcagaaagctgaaaatgaaaacactggaatctgtacaaacaaaagaggtagcagcaatccattgcttacaactgaagaggcaaatttgacagagaaagaggaaataaggcaaggtgaaacactgatgatagaaggaacagaacagttgaaatctctctcttcagactcttcattttgctttcccaggcctcacttctcattctccactttgccaactgtttcaagaactgtggaactcaaatcagaacctaatgtcatcagttctcctgctgagtgttccttggaactttctccttcaaggccttgtgttttacattcttcactctctaggagagagacacctatttgtatgttacctattgaaaccgaaagaaatatttttgaaaattttgcccatccaccaaacatctctccttctgcctgtccccttccccctcctcctcctatttctcctccttctcctcctcctgctcctgctcctcttgctcctcctcctgacatttctcctttttctcttttttgtcctcctccctctcctccttctatccctcttcctcttcctcctcctacattttttccactttccgtttcaacgtctggtcccccaacaccacctcttctacctccatttccaactcctcttcctccaccacctccttctattccttgccctccacctccttcagcttcatttctgtccacagagtgtgtctgtataacaggtgttaaatgcacgaccaacttgatgcctgccgagaaaattaagtcctctatgacacagctatcaacaacgacagtgtgtaaaacagaccctcagagagaaccaaaaggcatcctcagacacgttaaaaacttagcagaacttgaaaaatcagtagctaacatgtacagtcaaatagaaaaaaactatctacgcacaaatgtttcagaacttcaaactatgtgcccttcagaagtaacaaatatggaaatcacatctgaacaaaacaaggggagtttgaacaatattgtcgagggaactgaaaaacaatctcacagtcaatctacttcactgtaa

SEQ ID NO: 2 - protein, 1962 aa, Homo sapiens
MFRQFYLWTCLASGIILGSLFEICLGQYDDDWQYEDCKLARGGPPATIVAIDEESRNGTILVDNMLIKGTAGGPDPTIELSLKDNVDYWVLMDPVKQMLFLNSTGRVLDRDPPMNIHSIVVQVQCINKKVGTIIYHEVRIVVRDRNDNSPTFKHESYYATVNELTPVGTTIFTGFSGDNGATDIDDGPNGQIEYVIQYNPDDPTSNDTFEIPLMLTGNIVLRKRLNYEDKTRYFVIIQANDRAQNLNERRTTTTTLTVDVLDGDDLGPMFLPCVLVPNTRDCRPLTYQAAIPELRTPEELNPIIVTPPIQAIDQDRNIQPPSDRPGILYSILVGTPEDYPRFFHMHPRTAELSLLEPVNRDFHQKFDLVIKAEQDNGHPLPAFAGLHIEILDENNQSPYFTMPSYQGYILESAPVGATISDSLNLTSPLRIVALDKDIEDTKDPELHLFLNDYTSVFTVTQTGITRYLTLLQPVDREEQQTYTFSITAFDGVQESEPVIVNIQVMDANDNTPTFPEISYDVYVYTDMRPGDSVIQLTAVDADEGSNGEITYEILVGAQGDFIINKTTGLITIAPGVEMIVGRTYALTVQAADNAPPAERRNSICTVYIEVLPPNNQSPPRFPQLMYSLEISEAMRVGAVLLNLQATDREGDSITYAIENGDPQRVFNLSETTGILTLGKALDRESTDRYILIITASDGRPDGTSTATVNIVVTDVNDNAPVFDPYLPRNLSVVEEEANAFVGQVKATDPDAGINGQVHYSLGNFNNLFRITSNGSIYTAVKLNREVRDYYELVVVATDGAVHPRHSTLTLAIKVLDIDDNSPVFTNSTYTVLVEENLPAGTTILQIEAKDVDLGANVSYRIRSPEVKHFFALHPFTGELSLLRSLDYEAFPDQEASITFLVEAFDIYGTMPPGIATVTVIVKDMNDYPPVFSKRIYKGMVAPDAVKGTPITTVYAEDADPPGLPASRVRYRVDDVQFPYPASIFEVEEDSGRVITRVNLNEEPTTIFKLVVVAFDDGEPVMSSSATVKILVLHPGEIPRFTQEEYRPPPVSELATKGTMVGVISAAAINQSIVYSIVSGNEEDTFGINNITGVIYVNGPLDYETRTSYVLRVQADSLEVVLANLRVPSKSNTAKVYIEIQDENNHPPVFQKKFYIGGVSEDARMFTSVLRVKATDKDTGNYSVMAYRLIIPPIKEGKEGFVVETYTGLIKTAMLFHNMRRSYFKFQVIATDDYGKGLSGKADVLVSVVNQLDMQVIVSNVPPTLVEKKIEDLTEILDRYVQEQIPGAKVVVESIGARRHGDAFSLEDYTKCDLTVYAIDPQTNRAIDRNELFKFLDGKLLDINKDFQPYYGEGGRILEIRTPEAVTSIKKRGESLGYTEGALLALAFIIILCCIPAILVVLVSYRQFKVRQAECTKTARIQAALPAAKPAVPAPAPVAAPPPPPPPPPGAHLYEELGDSSMHNLFLLYHFQQSRGNNSVSEDRKHQQVVMPFSSNTIEAHKSAHVDGSLKSNKLKSARKFTFLSDEDDLSAHNPLYKENISQVSTNSDISQRTDFVDPFSPKIQAKSKSLRGPREKIQRLWSQSVSLPRRLMRKVPNRPEIIDLQQWQGTRQKAENENTGICTNKRGSSNPLLTTEEANLTEKEEIRQGETLMIEGTEQLKSLSSDSSFCFPRPHFSFSTLPTVSRTVELKSEPNVISSPAECSLELSPSRPCVLHSSLSRRETPICMLPIETERNIFENFAHPPNISPSACPLPPPPPISPPSPPPAPAPLAPPPDISPFSLFCPPPSPPSIPLPLPPPTFFPLSVSTSGPPTPPLLPPFPTPLPPPPPSIPCPPPPSASFLSTECVCITGVKCTTNLMPAEKIKSSMTQLSTTTVCKTDPQREPKGILRHVKNLAELEKSVANMYSQIEKNYLRTNVSELQTMCPSEVTNMEITSEQNKGSLNNIVEGTEKQSHSQSTSL

SEQ ID NO: 3 - DNA, 5373 bp, Homo sapiens
  source 1..5373: mol_type = other DNA
atgtttcgacagttttatctctggacatgtttagcttcagggatcatcctgggctctctctttgaaatctgcttgggccagtatgatgatgactggcaatatgaggattgcaaactagctaggggaggaccaccagctaccatagttgctattgatgaagaaagtcggaatggtacaattctggtggacaacatgctgatcaaagggactgctggaggaccagaccccaccatagaactttctttaaaggataatgtggattactgggtgttgatggatcctgttaagcaaatgcttttcctgaacagcaccggaagagttctggatagagatccaccgatgaacatacactccattgtggtgcaggtccagtgcatcaacaaaaaagtgggcactattatctaccatgaagtgcgaatagtggtgagagacaggaatgacaactcacccactttcaagcatgaaagctactatgccacagtgaatgagctcactccagttggtaccacaatattcacaggattttcaggagacaatggagctacagatatagatgatggaccaaatggacagatagagtatgttattcagtataatccagatgatccgacatccaatgacacctttgaaattcccctaatgttgactggaaatatagtgttaaggaagaggctcaactatgaagataagactcgctactttgtcataatccaagctaatgaccgtgcccaaaatctgaatgagaggcgaaccaccaccaccactctcacagtggatgttctggatggagatgacttgggtccaatgtttcttccttgtgtccttgtgccaaacactcgtgattgccgtccactcacttatcaagctgccatacctgagttgagaactccggaagaactgaaccccattattgttacgccaccaatccaagccattgatcaggaccggaatattcaaccgccatcagataggccaggaatcctctattccatccttgttgggactcctgaggattacccacgatttttccatatgcatcctaggacagcagaacttagtctcctggagccagtaaacagagactttcaccagaaatttgatttggttattaaggctgaacaagacaatggtcatcctcttcctgcctttgccggtctacacattgaaatactggatgaaaacaatcaaagtccatattttacaatgcccagttatcaaggctatatcctggaatctgccccagtgggagcaaccatttcggacagtctcaatttgacttcacctttaagaatagtagctctggacaaggacatagaagatgttccacccagtggagttcctacaaaagacccagagcttcacctttttctgaatgactacacctcagtcttcaccgtcacacagactggtattactcgctacctcaccttacttcaaccagtggacagggaagaacagcaaacttacaccttttcgataacagcatttgatggtgtacaagaaagtgagccagtcatcgtcaatattcaagtgatggatgcaaatgataacacgccaaccttccctgaaatatcctatgatgtgtatgtttatacagacatgagacctggggacagtgtcatacagctcactgcagtcgacgcagacgaagggtcaaatggggagatcacatatgaaatccttgttggggctcagggagacttcatcatcaataaaacaacagggcttatcaccatcgctccaggggtggaaatgatagtcgggcggacttacgcactcacggtccaagcagcggataatgctcctcctgcagagcgaaggaactccatctgcactgtgtatattgaagtgcttccaccaaataatcaaagccctcctcgcttcccacagctgatgtatagccttgaaattagtgaagccatgagggttggtgctgttttattaaatctacaggcaactgatcgagagggagactcaataacatatgccattgagaatggagatcctcagagagtttttaatctttcagaaaccacggggattctaaccttagggaaagcactggacagggaaagcactgatcgctacattctgatcatcacagcttcagatggcaggccagatgggacctcaactgccacagtaaacatagtggtgacagatgtcaatgacaatgctccagtgtttgatccttatctgccaagaaatttatctgtggtggaagaagaagccaatgcctttgtgggtcaagtaaaagcaacagaccctgatgctggaataaatggtcaagtgcactacagtttgggtaactttaataatctttttcgtatcacatccaatgggagcatttacacagcagtgaagcttaacagagaagtcagggactactatgaacttgttgttgtggcaacagatggagcagtacaccctcgtcattcaactctaaccttggccatcaaggttttggacattgatgataacagtcctgtgttcaccaattcaacatacactgtccttgttgaagagaatttgccagctgggactaccatccttcaaatagaggccaaagatgtcgaccttggagcaaatgtgtcttaccggataagaagcccagaagtgaagcacttttttgcactacatccatttacaggagaactatcgcttttaaggagtttagattatgaggcatttccagaccaagaagcaagtatcacttttctggtagaggcctttgatatttatggaacaatgccacctggtattgctactgtcacagtgattgtaaaggatatgaatgattatcctcctgtctttagtaaacgaatatacaaagggatggtggctccggatgcagtcaagggtacacctatcacaacagtttatgctgaagatgcagaccctcctggattacctgcaagtcgtgtgaggtatagagtagatgatgtacagtttccttaccctgccagtatttttgaagtggaagaagattctggaagagtaataacacgagtcaatcttaatgaagaacctacaacaatttttaagttggtggtggttgcttttgatgatggggagcctgtgatgtccagcagtgccacagtgaagattcttgtcttacatcctggtgagatcccacgcttcacacaggaggaatatagacctcctccagtaagtgaacttgccaccaaagggaccatggttggtgtaatttctgctgctgccattaatcaaagtattgtgtactccattgtttcaggaaatgaagaagatacatttggaattaataacatcacaggtgttatctatgtgaatggacctctggattatgagaccaggacaagctatgtacttcgagtccaagctgattccctggaagtggtccttgccaatctccgagttccttcaaaaagcaatacagctaaagtatacattgagattcaggatgaaaataatcatcccccagtgtttcagaaaaaattctacatcggaggtgtatctgaagatgcaagaatgtttacttctgtactcagagtgaaggctactgataaagatactggcaattatagtgtcatggcctacagactcataataccaccaattaaagagggaaaagaaggatttgtagtggaaacatatacagggcttatcaaaactgctatgctcttccataatatgaggagatcctacttcaagtttcaagttattgcaactgacgactatgggaagggactgagcggcaaagccgatgtactcgtctccgtggtcaatcagctggatatgcaagtcattgtttccaatgtgcctcctactctagtggaaaaaaagatagaagatcttacagagatcttggatcgctatgttcaggaacaaattcctggtgccaaggtcgtagtggagtccattggagctcgccggcatggagatgccttttccctagaagattacaccaaatgtgacttgactgtctatgcaattgacccccaaaccaacagagccatcgatagaaatgagctttttaaatttttggatggcaaactacttgatatcaataaagactttcagccgtattatggggaaggaggacgcattctggagatccggactccagaggcagtgaccagcattaaaaagagaggagaaagtctaggatacacagaaggggccttgttggctctggccttcatcatcatcctctgctgcattcctgccatcttggtggttttggtcagctacagacagtttaaagtacgtcaagctgagtgtacaaagactgcacgaattcaggccgcattacccgcggctaaaccagcagtgccggctcctgcaccagtggcagcgcccccgccgccgccgccgcctccgccaggtgcgcatctctatgaagaacttggagacagctcaatgcataagtatgaaatgcctcaatatgggagtcgccgtcgattgttaccaccagctggacaggaggaatatggtgaggtggttggtgaagctgaggaagaatatgaggaggaagaggaagagccaaagaaaattaaaaaaccaaaggttgaaattagagagcctagtgaggaggaagaagtagttgtaactatcgaaaaaccaccagcagctgagcctacatacacaacatggaagagagccagaatattccccatgatttttaagaaagttagaggattagctgataaaagaggaatcgttgaccttgagggtgaagagtggcagagacgccttgaggaagaagataaagattatttgaaactcactctggaccaagaggaagcaacagaaagcactgtagaatcagaggaggaatcctccagcgactatactgaatacagtgaagaagagtctgagttcagtgagtctgagactacagaagaggaatctgagtcagagacaccctctgaggaggaggagagttccacccctgaatcagaagaatcggaatccacagagtcagaaggagaaaaagcaaggaaaaacattgtgcttgcaagaagaaggcccatggttgaggaggtcaaggaagtcaagggtaggaaagaggagccacaagaagaacaaaaagaacctaagatggaagaagaagaacactcagaagaagaagaaagtggaccagcccctgtggaagaaagtacagaccctgaagctcaagatatccctgaagagggcagtgcagaatcagcttcggtggaaggaggtgtggaaagtgaggaggaatcagaatcaggtagtagtagcagtagtagcgaaagtcagtctggaggtccatggggctatcaggtaccagcgtatgacagaagcaagaatgcaaaccaaaagaagtcgccaggagcaaactctgaaggttacaacacagcactttga

SEQ ID NO: 4 - protein, 1790 aa, Homo sapiens
MFRQFYLWTCLASGIILGSLFEICLGQYDDDWQYEDCKLARGGPPATIVAIDEESRNGTILVDNMLIKGTAGGPDPTIELSLKDNVDYWVLMDPVKQMLFLNSTGRVLDRDPPMNIHSIVVQVQCINKKVGTIIYHEVRIVVRDRNDNSPTFKHESYYATVNELTPVGTTIFTGFSGDNGATDIDDGPNGQIEYVIQYNPDDPTSNDTFEIPLMLTGNIVLRKRLNYEDKTRYFVIIQANDRAQNLNERRTTTTTLTVDVLDGDDLGPMFLPCVLVPNTRDCRPLTYQAAIPELRTPEELNPIIVTPPIQAIDQDRNIQPPSDRPGILYSILVGTPEDYPRFFHMHPRTAELSLLEPVNRDFHQKFDLVIKAEQDNGHPLPAFAGLHIEILDENNQSPYFTMPSYQGYILESAPVGATISDSLNLTSPLRIVALDKDIEDVPPSGVPTKDPELHLFLNDYTSVFTVTQTGITRYLTLLQPVDREEQQTYTFSITAFDGVQESEPVIVNIQVMDANDNTPTFPEISYDVYVYTDMRPGDSVIQLTAVDADEGSNGEITYEILVGAQGDFIINKTTGLITIAPGVEMIVGRTYALTVQAADNAPPAERRNSICTVYIEVLPPNNQSPPRFPQLMYSLEISEAMRVGAVLLNLQATDREGDSITYAIENGDPQRVFNLSETTGILTLGKALDRESTDRYILIITASDGRPDGTSTATVNIVVTDVNDNAPVFDPYLPRNLSVVEEEANAFVGQVKATDPDAGINGQVHYSLGNFNNLFRITSNGSIYTAVKLNREVRDYYELVVVATDGAVHPRHSTLTLAIKVLDIDDNSPVFTNSTYTVLVEENLPAGTTILQIEAKDVDLGANVSYRIRSPEVKHFFALHPFTGELSLLRSLDYEAFPDQEASITFLVEAFDIYGTMPPGIATVTVIVKDMNDYPPVFSKRIYKGMVAPDAVKGTPITTVYAEDADPPGLPASRVRYRVDDVQFPYPASIFEVEEDSGRVITRVNLNEEPTTIFKLVVVAFDDGEPVMSSSATVKILVLHPGEIPRFTQEEYRPPPVSELATKGTMVGVISAAAINQSIVYSIVSGNEEDTFGINNITGVIYVNGPLDYETRTSYVLRVQADSLEVVLANLRVPSKSNTAKVYIEIQDENNHPPVFQKKFYIGGVSEDARMFTSVLRVKATDKDTGNYSVMAYRLIIPPIKEGKEGFVVETYTGLIKTAMLFHNMRRSYFKFQVIATDDYGKGLSGKADVLVSVVNQLDMQVIVSNVPPTLVEKKIEDLTEILDRYVQEQIPGAKVVVESIGARRHGDAFSLEDYTKCDLTVYAIDPQTNRAIDRNELFKFLDGKLLDINKDFQPYYGEGGRILEIRTPEAVTSIKKRGESLGYTEGALLALAFIIILCCIPAILVVLVSYRQFKVRQAECTKTARIQAALPAAKPAVPAPAPVAAPPPPPPPPPGAHLYEELGDSSMHKYEMPQYGSRRRLLPPAGQEEYGEVVGEAEEEYEEEEEEPKKIKKPKVEIREPSEEEEVVVTIEKPPAAEPTYTTWKRARIFPMIFKKVRGLADKRGIVDLEGEEWQRRLEEEDKDYLKLTLDQEEATESTVESEEESSSDYTEYSEEESEFSESETTEEESESETPSEEEESSTPESEESESTESEGEKARKNIVLARRRPMVEEVKEVKGRKEEPQEEQKEPKMEEEEHSEEEESGPAPVEESTDPEAQDIPEEGSAESASVEGGVESEEESESGSSSSSSESQSGGPWGYQVPAYDRSKNANQKKSPGANSEGYNTAL

SEQ ID NO: 5 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 6 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 7 - DNA, 523 bp, synthetic construct
  source 1..523: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgt

SEQ ID NO: 8 - DNA, 293 bp, synthetic construct
  source 1..293: mol_type = other DNA
gtcgacgcagggcagcctctgtcatctccatcagggaggggtccagtgtggagtctcggtggatctcgtatttcatgtctccaggctcaaagagacccatgagatgggtcacagacgggtccagggaagcatgcatgagctcagtgcggttccacacataccgggcaccctggcgcttcgccagccattcctgcaccagattcttcccgtccagcctggtcccaccttggctgtagtcatctgggtactcagggtctggggttcccatgcgaaacatgtactttcggcctcca

SEQ ID NO: 9 - DNA, 106 bp, synthetic construct
  source 1..106: mol_type = other DNA
gtaagtatcaaggttacaagacaggtttaaggagaccaatagaaactgggcttgtcgagacagagaagactcttgcgtttctgaggtaccgcatgcaccacggacc

SEQ ID NO: 10 - DNA, 291 bp, synthetic construct
  source 1..291: mol_type = other DNA
cgcagggcagcctctgtcatctccatcagggaggggtccagtgtggagtctcggtggatctcgtatttcatgtctccaggctcaaagagacccatgagatgggtcacagacgggtccagggaagcatgcatgagctcagtgcggttccacacataccgggcaccctggcgcttcgccagccattcctgcaccagattcttcccgtccagcctggtcccaccttggctgtagtcatctgggtactcagggtctggggttcccatgcgaaacatgtactttcggcctccagga

SEQ ID NO: 11 - DNA, 72 bp, synthetic construct
  source 1..72: mol_type = other DNA
cctaggatcacgcatgctcgcgataggcacctattggtcttactgacatccactttgcctttctctccacag

SEQ ID NO: 12 - DNA, 215 bp, synthetic construct
  source 1..215: mol_type = other DNA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 13 - DNA, 3635 bp, synthetic construct
  source 1..3635: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccgtttaaaccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtaggccgccaccatggatgtttcgacagttttatctctggacatgtttagcttcagggatcatcctgggctctctctttgaaatctgcttgggccagtatgatgatgactggcaatatgaggattgcaaactagctaggggaggaccaccagctaccatagttgctattgatgaagaaagtcggaatggtacaattctggtggacaacatgctgatcaaagggactgctggaggaccagaccccaccatagaactttctttaaaggataatgtggattactgggtgttgatggatcctgttaagcaaatgcttttcctgaacagcaccggaagagttctggatagagatccaccgatgaacatacactccattgtggtgcaggtccagtgcatcaacaaaaaagtgggcactattatctaccatgaagtgcgaatagtggtgagagacaggaatgacaactcacccactttcaagcatgaaagctactatgccacagtgaatgagctcactccagttggtaccacaatattcacaggattttcaggagacaatggagctacagatatagatgatggaccaaatggacagatagagtatgttattcagtataatccagatgatccgacatccaatgacacctttgaaattcccctaatgttgactggaaatatagtgttaaggaagaggctcaactatgaagataagactcgctactttgtcataatccaagctaatgaccgtgcccaaaatctgaatgagaggcgaaccaccaccaccactctcacagtggatgttctggatggagatgacttgggtccaatgtttcttccttgtgtccttgtgccaaacactcgtgattgccgtccactcacttatcaagctgccatacctgagttgagaactccggaagaactgaaccccattattgttacgccaccaatccaagccattgatcaggaccggaatattcaaccgccatcagataggccaggaatcctctattccatccttgttgggactcctgaggattacccacgatttttccatatgcatcctaggacagcagaacttagtctcctggagccagtaaacagagactttcaccagaaatttgatttggttattaaggctgaacaagacaatggtcatcctcttcctgcctttgccggtctacacattgaaatactggatgaaaacaatcaaagtccatattttacaatgcccagttatcaaggctatatcctggaatctgccccagtgggagcaaccatttcggacagtctcaatttgacttcacctttaagaatagtagctctggacaaggacatagaagatacaaaagacccagagcttcacctttttctgaatgactacacctcagtcttcaccgtcacacagactggtattactcgctacctcaccttacttcaaccagtggacagggaagaacagcaaacttacaccttttcgataacagcatttgatggtgtacaagaaagtgagccagtcatcgtcaatattcaagtgatggatgcaaatgataacacgccaaccttccctgaaatatcctatgatgtgtatgtttatacagacatgagacctggggacagtgtcatacagctcactgcagtcgacgcagacgaagggtcaaatggggagatcacatatgaaatccttgttggggctcagggagacttcatcatcaataaaacaacagggcttatcaccatcgctccaggggtggaaatgatagtcgggcggacttacgcactcacggtccaagcagcggataatgctcctcctgcagagcgaaggaactccatctgcactgtgtatattgaagtgcttccaccaaataatcaaagccctcctcgcttcccacagctgatgtatagccttgaaattagtgaagccatgagggttggtgctgttttattaaatctacaggcaactgatcgagagggagactcaataacatatgccattgagaatggagatcctcagagagtttttaatctttcagaaaccacggggattctaaccttagggaaagcactggacagggaaagcactgatcgctacattctgatcatcacagcttcagatggcaggccagatgggacctcaactgccacagtaaacatagtggtgacagatgtcaatgacaatgctccagtgtttgatccttatctgccaagaaatttatctgtggtaagtatcaaggttacaagacaggtttaaggagaccaatagaaactgggcttgtcgagacagagaagactcttgcgtttctgaggtaccgcatgcaccacggaccgtcgacgcagggcagcctctgtcatctccatcagggaggggtccagtgtggagtctcggtggatctcgtatttcatgtctccaggctcaaagagacccatgagatgggtcacagacgggtccagggaagcatgcatgagctcagtgcggttccacacataccgggcaccctggcgcttcgccagccattcctgcaccagattcttcccgtccagcctggtcccaccttggctgtagtcatctgggtactcagggtctggggttcccatgcgaaacatgtactttcggcctccaggatcctagagcatggctaagatctcctatgctaggctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcg

SEQ ID NO: 14 - DNA, 4740 bp, synthetic construct
  source 1..4740: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcttaattaacgcagggcagcctctgtcatctccatcagggaggggtccagtgtggagtctcggtggatctcgtatttcatgtctccaggctcaaagagacccatgagatgggtcacagacgggtccagggaagcatgcatgagctcagtgcggttccacacataccgggcaccctggcgcttcgccagccattcctgcaccagattcttcccgtccagcctggtcccaccttggctgtagtcatctgggtactcagggtctggggttcccatgcgaaacatgtactttcggcctccaggacctaggatcacgcatgctcgcgataggcacctattggtcttactgacatccactttgcctttctctccacaggtggaagaagaagccaatgcctttgtgggtcaagtaaaagcaacagaccctgatgctggaataaatggtcaagtgcactacagtttgggtaactttaataatctttttcgtatcacatccaatgggagcatttacacagcagtgaagcttaacagagaagtcagggactactatgaacttgttgttgtggcaacagatggagcagtacaccctcgtcattcaactctaaccttggccatcaaggttttggacattgatgataacagtcctgtgttcaccaattcaacatacactgtccttgttgaagagaatttgccagctgggactaccatccttcaaatagaggccaaagatgtcgaccttggagcaaatgtgtcttaccggataagaagcccagaagtgaagcacttttttgcactacatccatttacaggagaactatcgcttttaaggagtttagattatgaggcatttccagaccaagaagcaagtatcacttttctggtagaggcctttgatatttatggaacaatgccacctggtattgctactgtcacagtgattgtaaaggatatgaatgattatcctcctgtctttagtaaacgaatatacaaagggatggtggctccggatgcagtcaagggtacacctatcacaacagtttatgctgaagatgcagaccctcctggattacctgcaagtcgtgtgaggtatagagtagatgatgtacagtttccttaccctgccagtatttttgaagtggaagaagattctggaagagtaataacacgagtcaatcttaatgaagaacctacaacaatttttaagttggtggtggttgcttttgatgatggggagcctgtgatgtccagcagtgccacagtgaagattcttgtcttacatcctggtgagatcccacgcttcacacaggaggaatatagacctcctccagtaagtgaacttgccaccaaagggaccatggttggtgtaatttctgctgctgccattaatcaaagtattgtgtactccattgtttcaggaaatgaagaagatacatttggaattaataacatcacaggtgttatctatgtgaatggacctctggattatgagaccaggacaagctatgtacttcgagtccaagctgattccctggaagtggtccttgccaatctccgagttccttcaaaaagcaatacagctaaagtatacattgagattcaggatgaaaataatcatcccccagtgtttcagaaaaaattctacatcggaggtgtatctgaagatgcaagaatgtttacttctgtactcagagtgaaggctactgataaagatactggcaattatagtgtcatggcctacagactcataataccaccaattaaagagggaaaagaaggatttgtagtggaaacatatacagggcttatcaaaactgctatgctcttccataatatgaggagatcctacttcaagtttcaagttattgcaactgacgactatgggaagggactgagcggcaaagccgatgtactcgtctccgtggtcaatcagctggatatgcaagtcattgtttccaatgtgcctcctactctagtggaaaaaaagatagaagatcttacagagatcttggatcgctatgttcaggaacaaattcctggtgccaaggtcgtagtggagtccattggagctcgccggcatggagatgccttttccctagaagattacaccaaatgtgacttgactgtctatgcaattgacccccaaaccaacagagccatcgatagaaatgagctttttaaatttttggatggcaaactacttgatatcaataaagactttcagccgtattatggggaaggaggacgcattctggagatccggactccagaggcagtgaccagcattaaaaagagaggagaaagtctaggatacacagaaggggccttgttggctctggccttcatcatcatcctctgctgcattcctgccatcttggtggttttggtcagctacagacagtttaaagtacgtcaagctgagtgtacaaagactgcacgaattcaggccgcattacccgcggctaaaccagcagtgccggctcctgcaccagtggcagcgcccccgccgccgccgccgcctccgccaggtgcgcatctctatgaagaacttggagacagctcaatgcataatcttttccttctctaccattttcaacaaagcaggggaaataactcagtctcagaagacaggaaacatcaacaagttgtgatgcccttttcttccaatactattgaggctcacaagtcagctcatgtagacggatcacttaagagcaacaaactgaagtctgcaagaaaattcacatttctatctgatgaggatgacttaagtgcccataatcccctttataaggaaaacataagtcaagtatcaacaaattcagacatttcacagagaacagattttgtagacccattttcacccaaaatacaagccaagagtaagtctctgaggggcccaagagaaaagattcagaggctgtggagtcagtcagtcagcttacccaggaggctgatgaggaaagttccaaatagaccagagatcatagatctgcagcagtggcaaggcaccaggcagaaagctgaaaatgaaaacactggaatctgtacaaacaaaagaggtagcagcaatccattgcttacaactgaagaggcaaatttgacagagaaagaggaaataaggcaaggtgaaacactgatgatagaaggaacagaacagttgaaatctctctcttcagactcttcattttgctttcccaggcctcacttctcattctccactttgccaactgtttcaagaactgtggaactcaaatcagaacctaatgtcatcagttctcctgctgagtgttccttggaactttctccttcaaggccttgtgttttacattcttcactctctaggagagagacacctatttgtatgttacctattgaaaccgaaagaaatatttttgaaaattttgcccatccaccaaacatctctccttctgcctgtccccttccccctcctcctcctatttctcctccttctcctcctcctgctcctgctcctcttgctcctcctcctgacatttctcctttttctcttttttgtcctcctccctctcctccttctatccctcttcctcttcctcctcctacattttttccactttccgtttcaacgtctggtcccccaacaccacctcttctacctccatttccaactcctcttcctccaccacctccttctattccttgccctccacctccttcagcttcatttctgtccacagagtgtgtctgtataacaggtgttaaatgcacgaccaacttgatgcctgccgagaaaattaagtcctctatgacacagctatcaacaacgacagtgtgtaaaacagaccctcagagagaaccaaaaggcatcctcagacacgttaaaaacttagcagaacttgaaaaatcagtagctaacatgtacagtcaaatagaaaaaaactatctacgcacaaatgtttcagaacttcaaactatgtgcccttcagaagtaacaaatatggaaatcacatctgaacaaaacaaggggagtttgaacaatattgtcgagggaactgaaaaacaatctcacagtcaatctacttcactgtaaactagtatttaaatcaattggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaagatctcctagtctaggctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcg

SEQ ID NO: 15 - DNA, 3346 bp, synthetic construct
  source 1..3346: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccgtttaaaccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtaggccgccaccatggatgtttcgacagttttatctctggacatgtttagcttcagggatcatcctgggctctctctttgaaatctgcttgggccagtatgatgatgactggcaatatgaggattgcaaactagctaggggaggaccaccagctaccatagttgctattgatgaagaaagtcggaatggtacaattctggtggacaacatgctgatcaaagggactgctggaggaccagaccccaccatagaactttctttaaaggataatgtggattactgggtgttgatggatcctgttaagcaaatgcttttcctgaacagcaccggaagagttctggatagagatccaccgatgaacatacactccattgtggtgcaggtccagtgcatcaacaaaaaagtgggcactattatctaccatgaagtgcgaatagtggtgagagacaggaatgacaactcacccactttcaagcatgaaagctactatgccacagtgaatgagctcactccagttggtaccacaatattcacaggattttcaggagacaatggagctacagatatagatgatggaccaaatggacagatagagtatgttattcagtataatccagatgatccgacatccaatgacacctttgaaattcccctaatgttgactggaaatatagtgttaaggaagaggctcaactatgaagataagactcgctactttgtcataatccaagctaatgaccgtgcccaaaatctgaatgagaggcgaaccaccaccaccactctcacagtggatgttctggatggagatgacttgggtccaatgtttcttccttgtgtccttgtgccaaacactcgtgattgccgtccactcacttatcaagctgccatacctgagttgagaactccggaagaactgaaccccattattgttacgccaccaatccaagccattgatcaggaccggaatattcaaccgccatcagataggccaggaatcctctattccatccttgttgggactcctgaggattacccacgatttttccatatgcatcctaggacagcagaacttagtctcctggagccagtaaacagagactttcaccagaaatttgatttggttattaaggctgaacaagacaatggtcatcctcttcctgcctttgccggtctacacattgaaatactggatgaaaacaatcaaagtccatattttacaatgcccagttatcaaggctatatcctggaatctgccccagtgggagcaaccatttcggacagtctcaatttgacttcacctttaagaatagtagctctggacaaggacatagaagatacaaaagacccagagcttcacctttttctgaatgactacacctcagtcttcaccgtcacacagactggtattactcgctacctcaccttacttcaaccagtggacagggaagaacagcaaacttacaccttttcgataacagcatttgatggtgtacaagaaagtgagccagtcatcgtcaatattcaagtgatggatgcaaatgataacacgccaaccttccctgaaatatcctatgatgtgtatgtttatacagacatgagacctggggacagtgtcatacagctcactgcagtcgacgcagacgaagggtcaaatggggagatcacatatgaaatccttgttggggctcagggagacttcatcatcaataaaacaacagggcttatcaccatcgctccaggggtggaaatgatagtcgggcggacttacgcactcacggtccaagcagcggataatgctcctcctgcagagcgaaggaactccatctgcactgtgtatattgaagtgcttccaccaaataatcaaagccctcctcgcttcccacagctgatgtatagccttgaaattagtgaagccatgagggttggtgctgttttattaaatctacaggcaactgatcgagagggagactcaataacatatgccattgagaatggagatcctcagagagtttttaatctttcagaaaccacggggattctaaccttagggaaagcactggacagggaaagcactgatcgctacattctgatcatcacagcttcagatggcaggccagatgggacctcaactgccacagtaaacatagtggtgacagatgtcaatgacaatgctccagtgtttgatccttatctgccaagaaatttatctgtggtaagtatcaaggttacaagacaggtttaaggagaccaatagaaactgggcttgtcgagacagagaagactcttgcgtttctgaggtaccgcatgcaccacggaccgggatcctagagcatggctaagatctcctatgctaggctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - DNA, 4452 bp, synthetic construct
  source 1..4452: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcttaattaacctaggatcacgcatgctcgcgataggcacctattggtcttactgacatccactttgcctttctctccacaggtggaagaagaagccaatgcctttgtgggtcaagtaaaagcaacagaccctgatgctggaataaatggtcaagtgcactacagtttgggtaactttaataatctttttcgtatcacatccaatgggagcatttacacagcagtgaagcttaacagagaagtcagggactactatgaacttgttgttgtggcaacagatggagcagtacaccctcgtcattcaactctaaccttggccatcaaggttttggacattgatgataacagtcctgtgttcaccaattcaacatacactgtccttgttgaagagaatttgccagctgggactaccatccttcaaatagaggccaaagatgtcgaccttggagcaaatgtgtcttaccggataagaagcccagaagtgaagcacttttttgcactacatccatttacaggagaactatcgcttttaaggagtttagattatgaggcatttccagaccaagaagcaagtatcacttttctggtagaggcctttgatatttatggaacaatgccacctggtattgctactgtcacagtgattgtaaaggatatgaatgattatcctcctgtctttagtaaacgaatatacaaagggatggtggctccggatgcagtcaagggtacacctatcacaacagtttatgctgaagatgcagaccctcctggattacctgcaagtcgtgtgaggtatagagtagatgatgtacagtttccttaccctgccagtatttttgaagtggaagaagattctggaagagtaataacacgagtcaatcttaatgaagaacctacaacaatttttaagttggtggtggttgcttttgatgatggggagcctgtgatgtccagcagtgccacagtgaagattcttgtcttacatcctggtgagatcccacgcttcacacaggaggaatatagacctcctccagtaagtgaacttgccaccaaagggaccatggttggtgtaatttctgctgctgccattaatcaaagtattgtgtactccattgtttcaggaaatgaagaagatacatttggaattaataacatcacaggtgttatctatgtgaatggacctctggattatgagaccaggacaagctatgtacttcgagtccaagctgattccctggaagtggtccttgccaatctccgagttccttcaaaaagcaatacagctaaagtatacattgagattcaggatgaaaataatcatcccccagtgtttcagaaaaaattctacatcggaggtgtatctgaagatgcaagaatgtttacttctgtactcagagtgaaggctactgataaagatactggcaattatagtgtcatggcctacagactcataataccaccaattaaagagggaaaagaaggatttgtagtggaaacatatacagggcttatcaaaactgctatgctcttccataatatgaggagatcctacttcaagtttcaagttattgcaactgacgactatgggaagggactgagcggcaaagccgatgtactcgtctccgtggtcaatcagctggatatgcaagtcattgtttccaatgtgcctcctactctagtggaaaaaaagatagaagatcttacagagatcttggatcgctatgttcaggaacaaattcctggtgccaaggtcgtagtggagtccattggagctcgccggcatggagatgccttttccctagaagattacaccaaatgtgacttgactgtctatgcaattgacccccaaaccaacagagccatcgatagaaatgagctttttaaatttttggatggcaaactacttgatatcaataaagactttcagccgtattatggggaaggaggacgcattctggagatccggactccagaggcagtgaccagcattaaaaagagaggagaaagtctaggatacacagaaggggccttgttggctctggccttcatcatcatcctctgctgcattcctgccatcttggtggttttggtcagctacagacagtttaaagtacgtcaagctgagtgtacaaagactgcacgaattcaggccgcattacccgcggctaaaccagcagtgccggctcctgcaccagtggcagcgcccccgccgccgccgccgcctccgccaggtgcgcatctctatgaagaacttggagacagctcaatgcataatcttttccttctctaccattttcaacaaagcaggggaaataactcagtctcagaagacaggaaacatcaacaagttgtgatgcccttttcttccaatactattgaggctcacaagtcagctcatgtagacggatcacttaagagcaacaaactgaagtctgcaagaaaattcacatttctatctgatgaggatgacttaagtgcccataatcccctttataaggaaaacataagtcaagtatcaacaaattcagacatttcacagagaacagattttgtagacccattttcacccaaaatacaagccaagagtaagtctctgaggggcccaagagaaaagattcagaggctgtggagtcagtcagtcagcttacccaggaggctgatgaggaaagttccaaatagaccagagatcatagatctgcagcagtggcaaggcaccaggcagaaagctgaaaatgaaaacactggaatctgtacaaacaaaagaggtagcagcaatccattgcttacaactgaagaggcaaatttgacagagaaagaggaaataaggcaaggtgaaacactgatgatagaaggaacagaacagttgaaatctctctcttcagactcttcattttgctttcccaggcctcacttctcattctccactttgccaactgtttcaagaactgtggaactcaaatcagaacctaatgtcatcagttctcctgctgagtgttccttggaactttctccttcaaggccttgtgttttacattcttcactctctaggagagagacacctatttgtatgttacctattgaaaccgaaagaaatatttttgaaaattttgcccatccaccaaacatctctccttctgcctgtccccttccccctcctcctcctatttctcctccttctcctcctcctgctcctgctcctcttgctcctcctcctgacatttctcctttttctcttttttgtcctcctccctctcctccttctatccctcttcctcttcctcctcctacattttttccactttccgtttcaacgtctggtcccccaacaccacctcttctacctccatttccaactcctcttcctccaccacctccttctattccttgccctccacctccttcagcttcatttctgtccacagagtgtgtctgtataacaggtgttaaatgcacgaccaacttgatgcctgccgagaaaattaagtcctctatgacacagctatcaacaacgacagtgtgtaaaacagaccctcagagagaaccaaaaggcatcctcagacacgttaaaaacttagcagaacttgaaaaatcagtagctaacatgtacagtcaaatagaaaaaaactatctacgcacaaatgtttcagaacttcaaactatgtgcccttcagaagtaacaaatatggaaatcacatctgaacaaaacaaggggagtttgaacaatattgtcgagggaactgaaaaacaatctcacagtcaatctacttcactgtaaactagtatttaaatcaattggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaagatctcctagtctaggctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 17 - DNA, 3635 bp, synthetic construct; identical to SEQ ID NO: 13
  source 1..3635: mol_type = other DNA

SEQ ID NO: 18 - DNA, 4198 bp, synthetic construct
  source 1..4198: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcttaattaacgcagggcagcctctgtcatctccatcagggaggggtccagtgtggagtctcggtggatctcgtatttcatgtctccaggctcaaagagacccatgagatgggtcacagacgggtccagggaagcatgcatgagctcagtgcggttccacacataccgggcaccctggcgcttcgccagccattcctgcaccagattcttcccgtccagcctggtcccaccttggctgtagtcatctgggtactcagggtctggggttcccatgcgaaacatgtactttcggcctccaggacctaggatcacgcatgctcgcgataggcacctattggtcttactgacatccactttgcctttctctccacaggtggaagaagaagccaatgcctttgtgggtcaagtaaaagcaacagaccctgatgctggaataaatggtcaagtgcactacagtttgggtaactttaataatctttttcgtatcacatccaatgggagcatttacacagcagtgaagcttaacagagaagtcagggactactatgaacttgttgttgtggcaacagatggagcagtacaccctcgtcattcaactctaaccttggccatcaaggttttggacattgatgataacagtcctgtgttcaccaattcaacatacactgtccttgttgaagagaatttgccagctgggactaccatccttcaaatagaggccaaagatgtcgaccttggagcaaatgtgtcttaccggataagaagcccagaagtgaagcacttttttgcactacatccatttacaggagaactatcgcttttaaggagtttagattatgaggcatttccagaccaagaagcaagtatcacttttctggtagaggcctttgatatttatggaacaatgccacctggtattgctactgtcacagtgattgtaaaggatatgaatgattatcctcctgtctttagtaaacgaatatacaaagggatggtggctccggatgcagtcaagggtacacctatcacaacagtttatgctgaagatgcagaccctcctggattacctgcaagtcgtgtgaggtatagagtagatgatgtacagtttccttaccctgccagtatttttgaagtggaagaagattctggaagagtaataacacgagtcaatcttaatgaagaacctacaacaatttttaagttggtggtggttgcttttgatgatggggagcctgtgatgtccagcagtgccacagtgaagattcttgtcttacatcctggtgagatcccacgcttcacacaggaggaatatagacctcctccagtaagtgaacttgccaccaaagggaccatggttggtgtaatttctgctgctgccattaatcaaagtattgtgtactccattgtttcaggaaatgaagaagatacatttggaattaataacatcacaggtgttatctatgtgaatggacctctggattatgagaccaggacaagctatgtacttcgagtccaagctgattccctggaagtggtccttgccaatctccgagttccttcaaaaagcaatacagctaaagtatacattgagattcaggatgaaaataatcatcccccagtgtttcagaaaaaattctacatcggaggtgtatctgaagatgcaagaatgtttacttctgtactcagagtgaaggctactgataaagatactggcaattatagtgtcatggcctacagactcataataccaccaattaaagagggaaaagaaggatttgtagtggaaacatatacagggcttatcaaaactgctatgctcttccataatatgaggagatcctacttcaagtttcaagttattgcaactgacgactatgggaagggactgagcggcaaagccgatgtactcgtctccgtggtcaatcagctggatatgcaagtcattgtttccaatgtgcctcctactctagtggaaaaaaagatagaagatcttacagagatcttggatcgctatgttcaggaacaaattcctggtgccaaggtcgtagtggagtccattggagctcgccggcatggagatgccttttccctagaagattacaccaaatgtgacttgactgtctatgcaattgacccccaaaccaacagagccatcgatagaaatgagctttttaaatttttggatggcaaactacttgatatcaataaagactttcagccgtattatggggaaggaggacgcattctggagatccggactccagaggcagtgaccagcattaaaaagagaggagaaagtctaggatacacagaaggggccttgttggctctggccttcatcatcatcctctgctgcattcctgccatcttggtggttttggtcagctacagacagtttaaagtacgtcaagctgagtgtacaaagactgcacgaattcaggccgcattacccgcggctaaaccagcagtgccggctcctgcaccagtggcagcgcccccgccgccgccgccgcctccgccaggtgcgcatctctatgaagaacttggagacagctcaatgcataagtatgaaatgcctcaatatgggagtcgccgtcgattgttaccaccagctggacaggaggaatatggtgaggtggttggtgaagctgaggaagaatatgaggaggaagaggaagagccaaagaaaattaaaaaaccaaaggttgaaattagagagcctagtgaggaggaagaagtagttgtaactatcgaaaaaccaccagcagctgagcctacatacacaacatggaagagagccagaatattccccatgatttttaagaaagttagaggattagctgataaaagaggaatcgttgaccttgagggtgaagagtggcagagacgccttgaggaagaagataaagattatttgaaactcactctggaccaagaggaagcaacagaaagcactgtagaatcagaggaggaatcctccagcgactatactgaatacagtgaagaagagtctgagttcagtgagtctgagactacagaagaggaatctgagtcagagacaccctctgaggaggaggagagttccacccctgaatcagaagaatcggaatccacagagtcagaaggagaaaaagcaaggaaaaacattgtgcttgcaagaagaaggcccatggttgaggaggtcaaggaagtcaagggtaggaaagaggagccacaagaagaacaaaaagaacctaagatggaagaagaagaacactcagaagaagaagaaagtggaccagcccctgtggaagaaagtacagaccctgaagctcaagatatccctgaagagggcagtgcagaatcagcttcggtggaaggaggtgtggaaagtgaggaggaatcagaatcaggtagtagtagcagtagtagcgaaagtcagtctggaggtccatggggctatcaggtaccagcgtatgacagaagcaagaatgcaaaccaaaagaagtcgccaggagcaaactctgaaggttacaacacagcactttgatatttaaatcaattggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaagatctcctagtctaggctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcg

SEQ ID NO: 19 - DNA, 3346 bp, synthetic construct; identical to SEQ ID NO: 15
  source 1..3346: mol_type = other DNA

SEQ ID NO: 20 - DNA, 3909 bp, synthetic construct
  source 1..3909: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcttaattaacctaggatcacgcatgctcgcgataggcacctattggtcttactgacatccactttgcctttctctccacaggtggaagaagaagccaatgcctttgtgggtcaagtaaaagcaacagaccctgatgctggaataaatggtcaagtgcactacagtttgggtaactttaataatctttttcgtatcacatccaatgggagcatttacacagcagtgaagcttaacagagaagtcagggactactatgaacttgttgttgtggcaacagatggagcagtacaccctcgtcattcaactctaaccttggccatcaaggttttggacattgatgataacagtcctgtgttcaccaattcaacatacactgtccttgttgaagagaatttgccagctgggactaccatccttcaaatagaggccaaagatgtcgaccttggagcaaatgtgtcttaccggataagaagcccagaagtgaagcacttttttgcactacatccatttacaggagaactatcgcttttaaggagtttagattatgaggcatttccagaccaagaagcaagtatcacttttctggtagaggcctttgatatttatggaacaatgccacctggtattgctactgtcacagtgattgtaaaggatatgaatgattatcctcctgtctttagtaaacgaatatacaaagggatggtggctccggatgcagtcaagggtacacctatcacaacagtttatgctgaagatgcagaccctcctggattacctgcaagtcgtgtgaggtatagagtagatgatgtacagtttccttaccctgccagtatttttgaagtggaagaagattctggaagagtaataacacgagtcaatcttaatgaagaacctacaacaatttttaagttggtggtggttgcttttgatgatggggagcctgtgatgtccagcagtgccacagtgaagattcttgtcttacatcctggtgagatcccacgcttcacacaggaggaatatagacctcctccagtaagtgaacttgccaccaaagggaccatggttggtgtaatttctgctgctgccattaatcaaagtattgtgtactccattgtttcaggaaatgaagaagatacatttggaattaataacatcacaggtgttatctatgtgaatggacctctggattatgagaccaggacaagctatgtacttcgagtccaagctgattccctggaagtggtccttgccaatctccgagttccttcaaaaagcaatacagctaaagtatacattgagattcaggatgaaaataatcatcccccagtgtttcagaaaaaattctacatcggaggtgtatctgaagatgcaagaatgtttacttctgtactcagagtgaaggctactgataaagatactggcaattatagtgtcatggcctacagactcataataccaccaattaaagagggaaaagaaggatttgtagtggaaacatatacagggcttatcaaaactgctatgctcttccataatatgaggagatcctacttcaagtttcaagttattgcaactgacgactatgggaagggactgagcggcaaagccgatgtactcgtctccgtggtcaatcagctggatatgcaagtcattgtttccaatgtgcctcctactctagtggaaaaaaagatagaagatcttacagagatcttggatcgctatgttcaggaacaaattcctggtgccaaggtcgtagtggagtccattggagctcgccggcatggagatgccttttccctagaagattacaccaaatgtgacttgactgtctatgcaattgacccccaaaccaacagagccatcgatagaaatgagctttttaaatttttggatggcaaactacttgatatcaataaagactttcagccgtattatggggaaggaggacgcattctggagatccggactccagaggcagtgaccagcattaaaaagagaggagaaagtctaggatacacagaaggggccttgttggctctggccttcatcatcatcctctgctgcattcctgccatcttggtggttttggtcagctacagacagtttaaagtacgtcaagctgagtgtacaaagactgcacgaattcaggccgcattacccgcggctaaaccagcagtgccggctcctgcaccagtggcagcgcccccgccgccgccgccgcctccgccaggtgcgcatctctatgaagaacttggagacagctcaatgcataagtatgaaatgcctcaatatgggagtcgccgtcgattgttaccaccagctggacaggaggaatatggtgaggtggttggtgaagctgaggaagaatatgaggaggaagaggaagagccaaagaaaattaaaaaaccaaaggttgaaattagagagcctagtgaggaggaagaagtagttgtaactatcgaaaaaccaccagcagctgagcctacatacacaacatggaagagagccagaatattccccatgatttttaagaaagttagaggattagctgataaaagaggaatcgttgaccttgagggtgaagagtggcagagacgccttgaggaagaagataaagattatttgaaactcactctggaccaagaggaagcaacagaaagcactgtagaatcagaggaggaatcctccagcgactatactgaatacagtgaagaagagtctgagttcagtgagtctgagactacagaagaggaatctgagtcagagacaccctctgaggaggaggagagttccacccctgaatcagaagaatcggaatccacagagtcagaaggagaaaaagcaaggaaaaacattgtgcttgcaagaagaaggcccatggttgaggaggtcaaggaagtcaagggtaggaaagaggagccacaagaagaacaaaaagaacctaagatggaagaagaagaacactcagaagaagaagaaagtggaccagcccctgtggaagaaagtacagaccctgaagctcaagatatccctgaagagggcagtgcagaatcagcttcggtggaaggaggtgtggaaagtgaggaggaatcagaatcaggtagtagtagcagtagtagcgaaagtcagtctggaggtccatggggctatcaggtaccagcgtatgacagaagcaagaatgcaaaccaaaagaagtcgccaggagcaaactctgaaggttacaacacagcactttgaatttaaatcaattggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaagatctcctagtctaggctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 21 - DNA, 23 bp, synthetic construct
  source 1..23: mol_type = other DNA
ccagaaaaggctcaacatcaact

SEQ ID NO: 22 - DNA, 20 bp, synthetic construct
  source 1..20: mol_type = other DNA
accaggacaacaccgtcaat

SEQ ID NO: 23 - DNA, 20 bp, synthetic construct
  source 1..20: mol_type = other DNA
gcagtctcccatctctacgc

SEQ ID NO: 24 - DNA, 20 bp, synthetic construct
  source 1..20: mol_type = other DNA
cgagtccgagtcatccaagg

SEQ ID NO: 25 - DNA, 20 bp, synthetic construct
  source 1..20: mol_type = other DNA
tgcaccaccaactgcttagc

SEQ ID NO: 26 - DNA, 21 bp, synthetic construct
  source 1..21: mol_type = other DNA
ggcatggactgtggtcatgag

SEQ ID NO: 27 - DNA, 22 bp, synthetic construct
  source 1..22: mol_type = other DNA
ctccaacatcctgaacctcagc

SEQ ID NO: 28 - DNA, 23 bp, synthetic construct
  source 1..23: mol_type = other DNA
cgtcacaccattgctattcttcg

SEQ ID NO: 29 - DNA, 20 bp, synthetic construct
  source 1..20: mol_type = other DNA
cactgaccacatcctctcgg

SEQ ID NO: 30 - DNA, 20 bp, synthetic construct
  source 1..20: mol_type = other DNA
gagggttcccgctttacctc

SEQ ID NO: 31 - DNA, 22 bp, synthetic construct
  source 1..22: mol_type = other DNA
cctgaagcagaagaggatcacc

SEQ ID NO: 32 - DNA, 22 bp, synthetic construct
  source 1..22: mol_type = other DNA
aaagcggcagatggtcgtttgg

SEQ ID NO: 33 - DNA, 22 bp, synthetic construct
  source 1..22: mol_type = other DNA
acatttaaactctggggcaggt

SEQ ID NO: 34 - DNA, 20 bp, synthetic construct
  source 1..20: mol_type = other DNA
gctgggttggtgtgtgagag

SEQ ID NO: 35 - DNA, 22 bp, synthetic construct
  source 1..22: mol_type = other DNA
tgtttgatccttatctgccaag

SEQ ID NO: 36 - DNA, 20 bp, synthetic construct
  source 1..20: mol_type = other DNA
ttccagcatcagggtctgtt

SEQ ID NO: 37 - DNA, 20 bp, synthetic construct
  source 1..20: mol_type = other DNA
gtctgtcggtcctgagcaaa

SEQ ID NO: 38 - DNA, 20 bp, synthetic construct
  source 1..20: mol_type = other DNA
atattgcgtcatgccagggt

SEQ ID NO: 39 - DNA, 20 bp, synthetic construct
  source 1..20: mol_type = other DNA
tttggagggcttctttgcca

SEQ ID NO: 40 - DNA, 20 bp, synthetic construct
  source 1..20: mol_type = other DNA
ttacacaccaccacgtaccg

SEQ ID NO: 41 - DNA, 20 bp, synthetic construct
  source 1..20: mol_type = other DNA
cctggagaagaggaaagaga

SEQ ID NO: 42 - DNA, 24 bp, synthetic construct
  source 1..24: mol_type = other DNA
ttgaggacctctgtgtatttgtca

SEQ ID NO: 43 - DNA, 22 bp, synthetic construct
  source 1..22: mol_type = other DNA
gctacagcatgatgcaggacca

SEQ ID NO: 44 - DNA, 22 bp, synthetic construct
  source 1..22: mol_type = other DNA
tctgcgagctggtcatggagtt

SEQ ID NO: 45 - DNA, 20 bp, synthetic construct
  source 1..20: mol_type = other DNA
ttcttgtgccttgagtggca

SEQ ID NO: 46 - DNA, 20 bp, synthetic construct
  source 1..20: mol_type = other DNA
atagaggcggcactcggata

SEQ ID NO: 47 - DNA, 20 bp, synthetic construct
  source 1..20: mol_type = other DNA
acgatcctgggtgtgcgtaa

SEQ ID NO: 48 - DNA, 20 bp, synthetic construct
  source 1..20: mol_type = other DNA
ccgatgcctcaactctccag

SEQ ID NO: 49 - DNA, 20 bp, synthetic construct
  source 1..20: mol_type = other DNA
gaaggcattcaacgaagccc

SEQ ID NO: 50 - DNA, 20 bp, synthetic construct
  source 1..20: mol_type = other DNA
atactccgccatgacactgc